(19) United States
(12) Patent Application Publication
(10) Pub. No.: US 2003/0139578 A1
(43) Pub. Date: Jul. 24, 2003

(54) Antibodies specifically binding PDE8A and PDE8B

(75) Inventors: Janice K. Au-Young, Brisbane, CA (US); Benjamin G. Cocks, Sunnyvale, CA (US); Roger T. Coleman, Sunnyvale, CA (US); Jeffrey J. Seilhamer, Los Altos Hills, CA (US); Douglas A. Fisher, Groton, CT (US)

Correspondence Address: INCYTE CORPORATION (formerly known as Incyte, Genomics, Inc.), 3160 PORTER DRIVE, PALO ALTO, CA, 94304, US

(73) Assignee: Incyte Genomics, Inc., Palo Alto, CA

(21) Appl. No.: 10/272,970

(22) Filed: Oct. 15, 2002

Related U.S. Application Data
(63) Continuation-in-part of application No. 09/454,060, filed on Dec. 2, 1999.
Application No. 09/454,060 is a division of application No. 09/255,748, filed on Feb. 23, 1999, now Pat. No. 6,080,548.
Application No. 09/255,748 is a division of application No. 08/974,565, filed on Nov. 19, 1997, now Pat. No. 5,932,423.
Application No. 08/974,565 is a continuation-in-part of application No. 08/624,663, filed on Mar. 25, 1996, now Pat. No. 5,798,246.

(51) Int. Cl. G01N 33/53; G01N 33/574; A61K 39/395; C07K 16/40

(52) U.S. Cl. 530/388.8; 424/155.1; 435/7.1; 435/7.23

(57) ABSTRACT

The invention provides a antibody that specifically binds human PDE8s and is used to diagnose, stage and treat prostate cancer.

This application is a continuation-in-part of co-pending U.S. Ser. No. 09/454,060 filed Dec. 2, 1999; which is a divisional of U.S. Pat. No. 6,080,548 filed Feb. 23, 1999; which is a divisional of U.S. Pat. No. 5,932,423 filed Nov. 19, 1997; which is a continuation-in-part of U.S. Pat. No. 5,798,246, entitled “Novel Cyclic Nucleotide Phosphodiesterases”, filed Mar. 25, 1996.

### FIELD OF THE INVENTION

The invention relates to antibodies that specifically binds human cyclic nucleotide phosphodiesterases 8A and 8B (PDE8s) that can be used in the early diagnosis, prognosis, treatment and evaluation of progression and treatment of neoplastic, immune and neuronal disorders such as prostate cancer, lung tumors and type 1 diabetes.

### BACKGROUND OF THE INVENTION

Antibodies, or immunoglobulins (Ig), are components of immune response expressed on the surface of or secreted into the circulation by B cells. An antibody is composed of two identical heavy chains (H-chains) and two identical light chains interlinked by disulfide bonds, and its function is to bind and neutralize foreign antigens. Based on the H-chain, antibodies are classified as IgA, IgD, IgE, IgG or IgM, and the most common class, IgG, is tetrameric while the other classes are variants or multimers of the basic structure.

Antibodies are best described in terms of their two functional domains. Antigen recognition is mediated by the antigen binding fragment (Fab) region of the antibody, and effector functions are mediated by the crystallizable fragment (Fc) region. The binding of antibody to antigen triggers destruction of the antigen by phagocytic white blood cells such as macrophages and neutrophils. These cells express surface Fc receptors that specifically bind to the Fc region of the antibody and allow phagocytic cells to destroy antibody-bound antigen. Fc receptors are single-pass transmembrane glycoproteins containing about 350 amino acids whose extracellular portion typically contains two or three Ig domains (Sears et al. (1990) J Immunol 144:371-378).

Cyclic nucleotides (cAMP and cGMP) function as intracellular second messengers to transduce extracellular signals from sources such as hormones, light, and neurotransmitters. Cyclic nucleotide phosphodiesterases (PDEs) degrade cyclic nucleotides to their corresponding monophosphates, thereby regulating the intracellular concentrations of cyclic nucleotides and their effects on signal transduction. At least seven families of mammalian PDEs have been identified based on substrate specificity and affinity, sensitivity to cofactors, and sensitivity to inhibitory drugs (Beavo (1995) Physiological Reviews 75:725-48). Most of these families contain distinct genes, many of which are expressed in different tissues as alternative splice variants. Within families, there are multiple isozymes and multiple splice variants of those isozymes. The existence of multiple PDE families, isozymes, and splice variants presents an opportunity for regulation of cyclic nucleotide levels and functions.

PDE1s are Ca²⁺/calmodulin dependent, are reported to contain three different genes, each of which appears to have at least two different splice variants, and have been found in the lung, heart, and brain. Some of the calmodulin-dependent PDEs are regulated in vitro by phosphorylation and dephosphorylation. Phosphorylation of PDE1 decreases the affinity of the enzyme for calmodulin, decreases PDE activity, and increases steady state levels of cAMP. PDE2s are cGMP stimulated PDEs that are localized in the brain and are thought to mediate the effects of cAMP on catecholamine secretion. PDE3s are one of the major families of PDEs present in vascular smooth muscle. PDE3s are inhibited by cGMP, have high specificity for cAMP as a substrate, and play a role in cardiac function. One isozyme of PDE3 is regulated by one or more insulin-dependent kinases.

PDE4s are the predominant isozymes in most inflammatory cells, and some PDE4s are activated by cAMP-dependent phosphorylation. PDE5s are thought to be cGMP specific, but may also affect cAMP function. High levels of PDE5s are found in most smooth muscle preparations, in platelets and in the kidney. PDE6s play a role in vision and are regulated by light and cGMP. PDE7, which has only one known member, is cAMP specific and most closely related to PDE4, although it is not inhibited by rolipram, a specific inhibitor of PDE4. A complete listing of PDE families 1-7, their localization and their physiological roles is given in Beavo (supra).

PDEs are composed of a catalytic domain of ˜270 amino acid, an N-terminal regulatory domain responsible for binding cofactors, and, in some cases, a C-terminal domain of unknown function. A conserved motif, HDXXHXGXXN (SEQ ID NO:25), has been identified in the catalytic domain of all PDEs. PDE families display approximately 30% amino acid identity within this catalytic domain, while within the same family, isozymes such as PDE4A and PDE4B typically display about 85-95% identity in this region. Within a family, there is extensive similarity (>60%) outside the catalytic domain, while across families, there is little or no sequence similarity.

Many immune and inflammatory responses are inhibited by agents that increase intracellular levels of cAMP (Verghese et al. (1995) Mol Pharmacol 47:1164-1171). A variety of diseases have been attributed to increased PDE activity and associated with decreased levels of cyclic nucleotides. A form of diabetes insipidus in the mouse has been associated with increased PDE4 activity, and an increase in low-Kₘ cAMP PDE activity has been reported in leukocytes of atopic patients. In humans, defects in PDEs have been associated with autosomal recessive retinitis pigmentosa, and PDE3 has been associated with cardiac disease. Many inhibitors of PDEs have been identified and have undergone clinical evaluation. PDE3 inhibitors are being developed as antithrombotic agents, antihypertensive agents, and as cardiotonic agents useful in the treatment of congestive heart failure. Rolipram, an inhibitor of PDE4, has been used in the treatment of depression and is undergoing evaluation as an anti-inflammatory agent. Since rolipram has been shown to inhibit lipopolysaccharide induced TNF-α, which, in turn, has been shown to enhance HIV-1 replication in vitro; rolipram may inhibit HIV-1 replication (Angel et al. (1995) AIDS 9:1137-44). Based on its ability to suppress the production of cytokines such as TNF α and β and interferon γ, rolipram has also been shown to be effective in the treatment of encephalomyelitis. Rolipram may also be effective in treating tardive dyskinesia and was effective in treating multiple sclerosis in an experimental animal model (Sommer et al. (1995) Nat Med 1:244-248; Sasaki et al. (1995) Eur J Pharmacol 282:71-76).

Theophylline, a nonspecific PDE inhibitor used in the treatment of bronchial asthma and other respiratory diseases, is believed to act on airway smooth muscle function and in an anti-inflammatory or immunomodulatory capacity (Banner and Page (1995) Eur Respir J 8:996-1000). Pentoxifylline is another nonspecific PDE inhibitor used in the treatment of intermittent claudication and diabetes-induced peripheral vascular disease. Pentoxifylline is also known to block TNF α production and may inhibit HIV-1 replication (Angel, supra).

PDEs have also been reported to effect cellular proliferation of a variety of cell types and have been implicated in various cancers. Bang et al. (1994; Proc Natl Acad Sci 91:5330-5334) reported that growth of prostate carcinoma cell lines DU 145 and LNCaP was inhibited by delivery of cAMP derivatives and phosphodiesterase inhibitors and observed a permanent conversion in phenotype from epithelial to neuronal morphology. Others have suggested that PDE inhibitors have the potential to regulate mesangial cell proliferation and lymphocyte proliferation (Matousovic et al. (1995) J Clin Invest 96:401-410; Joulain et al. (1995) J Lipid Mediat Cell Signal 11:63-79, respectively). Finally, Deonarain et al. (1994, Br J Cancer 70:786-94) describe a cancer treatment that involves intracellular delivery of phosphodiesterases to particular cellular compartments or tumors and results in cell death.

Antibodies are useful as diagnostics when the antigen which they specifically bind is differentially expressed in association with a human disorder. For some disorders, antibodies may also be promising as therapeutic agents because they mimic, stimulate or interact with the body's own immune responses and have few side effects. The production of an antibody that specifically binds human PDE8s satisfies a need in the art by providing a composition which is useful in the diagnosis, prognosis, treatment and evaluation of progression and treatment of neoplastic, immune and neuronal disorders.

### SUMMARY OF THE INVENTION

The invention is based on the production and identification of antibodies that binds human cyclic nucleotide phosphodiesterases (PDE8s) and are useful to diagnose, to stage, to treat, or to monitor the progression or treatment of neoplastic, immune and neuronal disorders and particularly to diagnose and stage prostate cancer. The invention provides a purified antibody that binds PDE8s having the amino acid sequence of SEQ ID NO:1 or SEQ ID NO:8 or an epitope comprising from about Q₆₃₅ to residue P₆₄₉ of SEQ ID NO:1, an epitope from residue S₂₂₀ to about residue R₂₃₉ of SEQ ID NO:1 or an epitope from about residue H₂₂₇ to about residue R₂₄₆ of SEQ ID NO:8 to the proteins having the amino acid sequences of SEQ ID NO:1 or SEQ ID NO:8.

In one embodiment, the antibody is a monoclonal antibody. In a second embodiment, the antibody is a polyclonal antibody. The invention also provides a composition comprising the antibody that specifically binds human PDE8s and a labeling moiety; a kit comprising the composition; an array element comprising the antibody or the composition; and a substrate upon which the antibody or the composition is immobilized.

The invention provides a method for using an antibody to detect expression of a protein in a sample, the method comprising combining the antibody with a sample under conditions for formation of antibody:protein complexes, and detecting complex formation, wherein complex formation indicates expression of the protein in the sample. In one aspect, the sample is selected from prostate, pancreas or lung. In a second aspect, the assay is selected from antibody arrays, enzyme-linked immunoadsorbent assays, fluorescence-activated cell sorting, protein arrays, radioimmunoassays, and western analysis. In a third aspect, complex formation is compared to at least one standard and is an early diagnostic of prostate cancer.

The invention provides a method for immunopurification of a protein comprising attaching an antibody to a substrate, exposing the antibody to a sample containing protein under conditions to allow antibody:protein complexes to form, dissociating the protein from the complex, and collecting purified protein. The invention further provide methods for using an antibody to assess efficacy or toxicity of a molecule or compound, the method comprising treating a sample containing the protein with a molecule or compound; contacting the proteins in the sample with the composition comprising an antibody that binds the PDE8 having the amino acid sequence of SEQ ID NO:1 or SEQ ID NO:8 or an epitope comprising from about Q₆₃₅ to residue P₆₄₉ of SEQ ID NO:1, an epitope from residue S₂₂₀ to about residue R₂₃₉ of SEQ ID NO:1 or an epitope from about residue H₂₂₇ to about residue R₂₄₆ of SEQ ID NO:8 and a labeling moiety under conditions for complex formation; and comparing the amount of complex formation in the treated sample with the amount of complex formation in an untreated sample, wherein a difference in complex formation indicates efficacy or toxicity of the molecule or compound. In one aspect, the sample is from prostate, pancreas or lung. In a second aspect, the sample is from a subject in need of treatment for adenofibromatous hyperplasia. In a third aspect, the sample is from a mammalian model system.

The invention provides a method for treating prostate cancer comprising administering to a subject in need of therapeutic intervention a therapeutic antibody that specifically binds the protein, a multispecific molecule that specifically binds the protein, or a composition comprising an antibody and a pharmaceutical or therapeutic agent. The invention also provides a method for delivering a pharmaceutical or therapeutic agent comprising attaching the pharmaceutical or therapeutic agent to an antibody or a multispecific molecule that binds the protein having the amino acid sequence of SEQ ID NO:1 or SEQ ID NO:8 and administering the antibody or multispecific molecule to a subject in need of therapeutic intervention, wherein the antibody or multispecific molecule delivers the pharmaceutical or therapeutic agent. In one aspect, the antibody is a monoclonal antibody. In a second aspect, the antibody or pharmaceutical agent is delivered to cells of the prostate.

The invention provides a method for using a protein to screen a plurality of antibodies to identify an antibody that specifically binds the protein comprising contacting a plurality of antibodies with the protein under conditions to form an antibody:protein complex, and dissociating the antibody from the antibody:protein complex, thereby obtaining antibody that specifically binds the protein. In one embodiment, the antibody is selected from an intact immunoglobulin molecule, a polyclonal antibody, a monoclonal antibody, a multispecific molecule, a chimeric antibody, a recombinant antibody, a humanized antibody, a single chain antibody, a Fab fragment, an F(ab′)₂ fragment, an Fv fragment, and an antibody-peptide fusion protein.

### BRIEF DESCRIPTION OF THE FIGURES AND TABLES

FIGS. 1A-1F show the amino acid sequence (SEQ ID NO:7) and nucleic acid sequence (SEQ ID NO:3) of PDE8A. This alignment (and those found in the next three figures) was produced using MACDNASIS PRO software (Hitachi Software Engineering South San Francisco Calif.).

FIG. 2 shows the amino acid sequence (SEQ ID NO:16) and nucleic acid sequence (SEQ ID NO:10) of PDE8B.

FIGS. 3A-3I show the amino acid sequence (SEQ ID NO:1) and nucleic acid sequence (SEQ ID NO:2) of PDE8A(E).

FIGS. 4A-4G show the amino acid sequence (SEQ ID NO:8) and nucleic acid sequence (SEQ ID NO:9) of PDE8B(E).

FIGS. 5A-5F show the amino acid sequence alignments among PDE8A (SEQ ID NO:7), PDE8B (SEQ ID NO:16), PDE8A(E) (SEQ ID NO:1), PDE8B(E) (SEQ ID NO:8), and rat PDE4A(GI 1705952; SEQ ID NO:17), produced using the MEGALIGN program of LASERGENE software (DNASTAR Madison Wis.).

FIG. 6 shows the double-reciprocal, Lineweaver-Burke plot for the activity of PDE8A(E) using cAMP as a substrate; the positive X axis reflects the reciprocal of the substrate (cAMP) concentration (1/S), and the positive Y axis reflects the reciprocal of the reaction velocity (1/V). Lineweaver-Burke analysis was performed according to Segal (1995, Enzyme Kinetics, John Wiley and Sons, New York N.Y., pp. 214-245)

FIG. 7 shows the dependence of PDE8A(E) activity on divalent cation concentration; the positive X axis reflects cation concentration (mM), and the positive Y axis reflects the percent hydrolysis of cAMP. Divalent cations tested were calcium chloride (CaCL₂; circles), magnesium chloride (MgCl₂; squares), and manganese chloride (MnCl₂; diamonds).

FIG. 8 shows the effect of various PDE inhibitors on the activity of PDE8A(E); the positive X axis reflects the concentration of inhibitor (M), and the positive Y axis reflects the percent inhibition of the enzyme.

### DESCRIPTION OF THE INVENTION

It is understood that this invention is not limited to the particular machines, materials and methods described. It is also to be understood that the terminology used herein is for the purpose of describing particular embodiments and is not intended to limit the scope of the present invention which will be limited only by the appended claims. As used herein, the singular forms “a”, “an”, and “the” may include plural reference unless the context clearly dictates otherwise. For example, a reference to “an antibody” includes a plurality of such antibodies known to those skilled in the art.

Unless defined otherwise, all technical and scientific terms used herein have the same meanings as commonly understood by one of ordinary skill in the art to which this invention belongs. All publications mentioned herein are cited for the purpose of describing and disclosing the cell lines, protocols, reagents and vectors which are reported in the publications and which might be used in connection with the invention. Nothing herein is to be construed as an admission that the invention is not entitled to antedate such disclosure by virtue of prior invention.

Definitions

“Antibody” refers to intact immunoglobulin molecule, a polyclonal antibody, a monoclonal antibody, a chimeric antibody, a recombinant antibody, a humanized antibody, single chain antibodies, a Fab fragment, an F(ab′)₂ fragment, an Fv fragment, and an antibody-peptide fusion protein.

“Antigenic determinant” refers to an antigenic or immunogenic epitope, structural feature, or region of an oligopeptide, peptide, or protein which is capable of inducing formation of an antibody that specifically binds the protein. Biological activity is not a prerequisite for immunogenicity.

“Array” refers to an ordered arrangement of at least two cDNAs, proteins, or antibodies on a substrate. At least one of the cDNAs, proteins, or antibodies represents a control or standard, and the other cDNA, protein, or antibody is of diagnostic or therapeutic interest. The arrangement of at least two and up to about 40,000 cDNAs, proteins, or antibodies on the substrate assures that the size and signal intensity of each labeled complex, formed between each cDNA and at least one nucleic acid, each protein and at least one ligand or antibody, or each antibody and at least one protein to which the antibody specifically binds, is individually distinguishable.

A “cancer” refers to an adenocarcinoma, leukemia, lymphoma, melanoma, myeloma, sarcoma, teratocarcinoma, and tumors of the adrenal gland, bladder, bone, bone marrow, brain, breast, cervix, colon, esophagus, gall bladder, ganglia, heart, kidney, liver, lung, muscle, ovary, pancreas, parathyroid, penis, pituitary gland, prostate, salivary glands, skin, small intestine, spleen, stomach, testis, thymus, thyroid, and uterus.

“cDNA” refers to an isolated polynucleotide, nucleic acid molecule, or any fragment thereof that contains from about 400 to about 5,000 nucleotides. It may have originated recombinantly or synthetically, may be double-stranded or single-stranded, may represent coding and noncoding 3′ or 5′ sequence, and generally lacks introns.

A “composition” refers to the polynucleotide and a labeling moiety; a purified protein and a pharmaceutical carrier or a heterologous, labeling or purification moiety; an antibody and a labeling moiety or pharmaceutical agent; and the like.

“Derivative” refers to a cDNA, protein, or antibody that has been subjected to chemical modification. Derivatization of a cDNA, protein or antibody can involve substitution of non-traditional; bases or amino acids or the replacement of a hydrogen by an acetyl, acyl, alkyl, amino, formyl, or morpholino group (for example, 5-methylcytosine or pegylated cysteine). Derivative molecules retain the biological activities of the naturally occurring molecules but may confer longer lifespan or enhanced activity.

“Differential expression” refers to an increased or upregulated or a decreased or downregulated expression as detected by absence, presence, or a significant, measurable change in the amount of messenger RNA or protein in a sample.

“Disorder” refers to conditions, diseases or syndromes in which PDE8s, or the mRNA encoding PDE8s, are differentially expressed; these include neoplastic, immune and neuronal disorders and particularly early stage prostate cancer, as it is preceeded by adenofibromatous hyperplasia, lung tumors and type 1 diabetes.

An “expression profile” is a representation of gene expression in a sample. A nucleic acid expression profile is produced using sequencing, hybridization, or amplification (PCR) technologies and mRNAs or cDNAs from a sample. A protein expression profile, although time delayed, mirrors the nucleic acid expression profile and may use antibody or protein arrays, enzyme-linked immunosorbent assays, fluorescence-activated cell sorting, spatial immobilization such as 2-dimensional polyacrylamide electrophoresis (2-D PAGE) for western analysis, and radioimmunoassays to detect protein expression in a sample. The nucleic acids, proteins, or antibodies may be used in solution or attached to a substrate, and their detection is based on methods and labeling moieties well known in the art. Expression profiles may be produced and evaluated by methods such as electronic northern analysis, guilt-by-association, and transcript imaging. Expression profiles produced using normal versus diseased tissue are preferred; of note is the correspondence between mRNA and protein expression as discussed by Zweiger (2001, Transducing the Genome. McGraw-Hill, San Francisco, Calif.) and Glavas et al. (2001; T cell activation upregulates cyclic nucleotide phosphodiesterases 8A1 and 7A3, Proc Natl Acad Sci 98:6319-6342) among others.

“Guilt-by-association” (GBA) is a method for identifying cDNAs or proteins that are associated with a specific disease, regulatory pathway, subcellular compartment, cell type, tissue type, or species by their highly significant co-expression with known markers or therapeutics.

“Isolated or “purified” refers to an antibody that is separated from its natural environment and is from about 60% free to about 90% free from other components with which it is naturally associated.

“Labeling moiety” refers to any reporter molecule including radionuclides, enzymes, fluorescent, chemiluminescent, or chromogenic agents, substrates, cofactors, inhibitors, or magnetic particles than can be attached to or incorporated into a polynucleotide, protein, or antibody. Visible labels and dyes include but are not limited to anthocyanins, β glucuronidase, biotin, BIODIPY, Coomassie blue, Cy3 and Cy5, 4,6-diamidino-2-phenylindole (DAPI), digoxigenin, fluorescein, FITC, gold, green fluorescent protein, lissamine, luciferase, phycoerythrin, rhodamine, spyro red, silver, streptavidin, and the like. Radioactive markers include radioactive forms of hydrogen, iodine, phosphorous, sulfur, and the like.

A “multispecific molecule” has multiple binding specificities, can bind at least two distinct epitopes or molecules, one of which may be a molecule on the surface of a cell. Antibodies can perform as or be a part of a multispecific molecule.

A “pharmaceutical agent” or “therapeutic agent” may be an antibody, an antisense or RNAi molecule, a multispecific molecule, a peptide, a protein, a radionuclide, a small drug molecule, a cytospecific or cytotoxic drug such as abrin, actinomyosin D, cisplatin, crotin, doxorubicin, 5-fluorouracil, methotrexate, ricin, vincristine, vinblastine, or any combination of these elements.

“Post-translational modification” of a antibody can involve lipidation, glycosylation, phosphorylation, acetylation, racemization, proteolytic cleavage, and the like. These processes may occur synthetically or biochemically.

“Protein” refers to a polypeptide or any portion thereof. A “portion” of a protein refers to that length of amino acid sequence which would retain at least one biological activity, a domain identified by PFAM or PRINTS analysis or an antigenic determinant of the protein identified using Kyte-Doolittle algorithms of the PROTEAN program (DNASTAR, Madison Wis.). An “oligopeptide” is an amino acid sequence of about five residues to seven residues that is used as part of a fusion protein to produce an antibody that specifically binds human PDE8s.

“PDE8s” refers to human cyclic nucleotide phosphodiesterases that are exactly or highly homologous (>90%) to the amino acid sequence of SEQ ID NOs:1 and 8 obtained from any species including bovine, ovine, porcine, murine, equine, and preferably the human species, and from any source, whether natural, synthetic, semi-synthetic, or recombinant.

“Sample” is used in its broadest sense and may comprise a bodily fluid such as ascites, blood, cerebrospinal fluid, lymph, semen, sputum, urine and the like; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue, a tissue biopsy, or a tissue print; buccal cells, skin, hair, a hair follicle; and the like.

“Specific binding” refers to a precise interaction between two molecules which is dependent upon their structure and in particular, their molecular side groups. For example, specific binding refers to the intercalation of a regulatory protein into the major groove of a DNA molecule or to the binding between an epitope of a protein and an antibody.

“Substrate” refers to any rigid or semi-rigid support to which polynucleotides, proteins, or antibodies are bound and includes magnetic or nonmagnetic beads, capillaries or other tubing, chips, fibers, filters, gels, membranes, plates, polymers, slides, wafers, and microparticles with a variety of surface forms including channels, columns, pins, pores, trenches, and wells.

A “transcript image” (TI) is a profile of gene transcription activity in a particular tissue at a particular time. TI provides assessment of the relative abundance of expressed polynucleotides in the cDNA libraries of an EST database as described in U.S. Pat. No. 5,840,484, incorporated herein by reference.

The Invention

The invention describes the production of antibodies that specifically bind the cyclic nucleotide phosphodiesterases, PDE8A(E) and PDE8B(E) and that can be used in the diagnosis, prognosis, treatment and evaluation of progression or treatment of neoplastic, immune and neuronal disorders in which the proteins or polynucleotides are differentially expressed, and in particular, in the diagnosis of adenofibromatous hyperplasia as an indicator of early stage prostate cancer, type 1 diabetes and lung tumors. As described in EXAMPLE 11, the amino acid sequences the pde8s were analyzed to determine regions of high immunogenicity that would produce antibodies. An epitope extending from about Q₆₃₅ to residue P649 of SEQ ID NO:1 was chosen to produce an antibody that would specifically bind PDE8A(E), PDE8A, PDE8B(E), and PDE8B (SEQ ID NOs:1,7,8, and 16). An epitope from about residue S₂₂₀ to about residue R₂₃₉ of SEQ ID NO:1 was chosen to produce an antibody that would specifically bind PDE8A(E) or PDE8A. An epitope from about residue H₂₂₇ to about residue R₂₄₆ of SEQ ID NO:8 was chosen to produce an an antibody that would specifically bind PDE8B(E), and PDE8B. At the time the original application was filed, PDE8A(E), PDE8A, PDE8B(E), and PDE8B appeared to constitute a new family of cyclic nucleotide phosphodiesterases designated PDE8s. Co-pending application U.S. Ser. No. 09/454,060, filed Dec. 2, 1999, is incorporated by reference herein in its entirety.

Nucleic acids encoding the PDE8A(E) (SEQ ID NO:2) of the present invention were first identified in Incyte cDNA clone 156196 (SEQ ID NO:3) from the promonocyte cell line cDNA library (THP1PLB02). SEQ ID NO:3 is a fragment of and has 98% identity to SEQ ID NO:2 with which it aligns from C₄₇₁-C₂₆₆₈. SEQ ID NO:2 was derived by extension of SEQ ID NO:3 as described in EXAMPLE III.

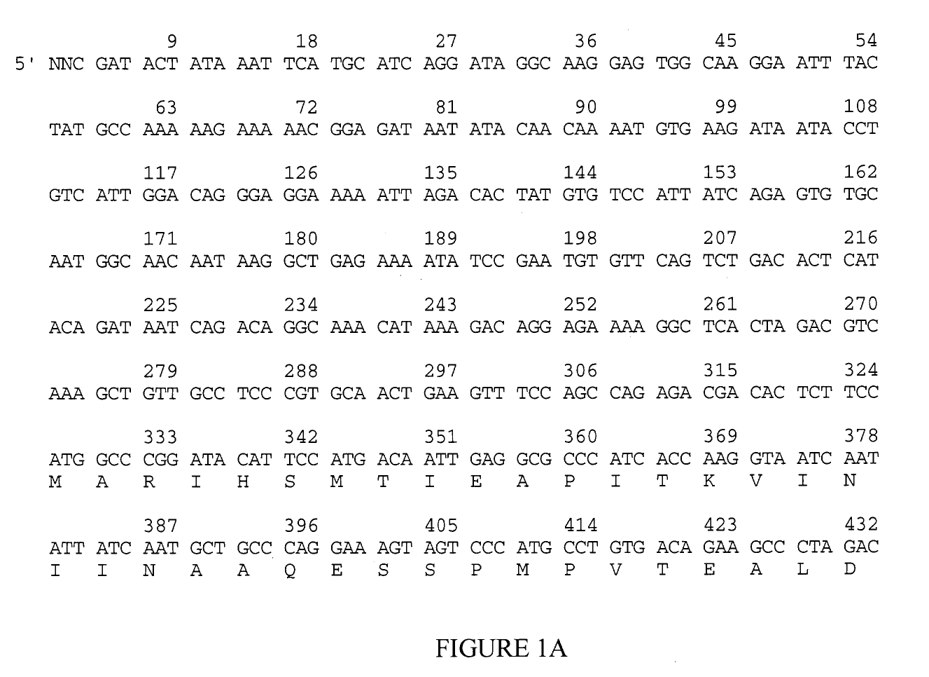

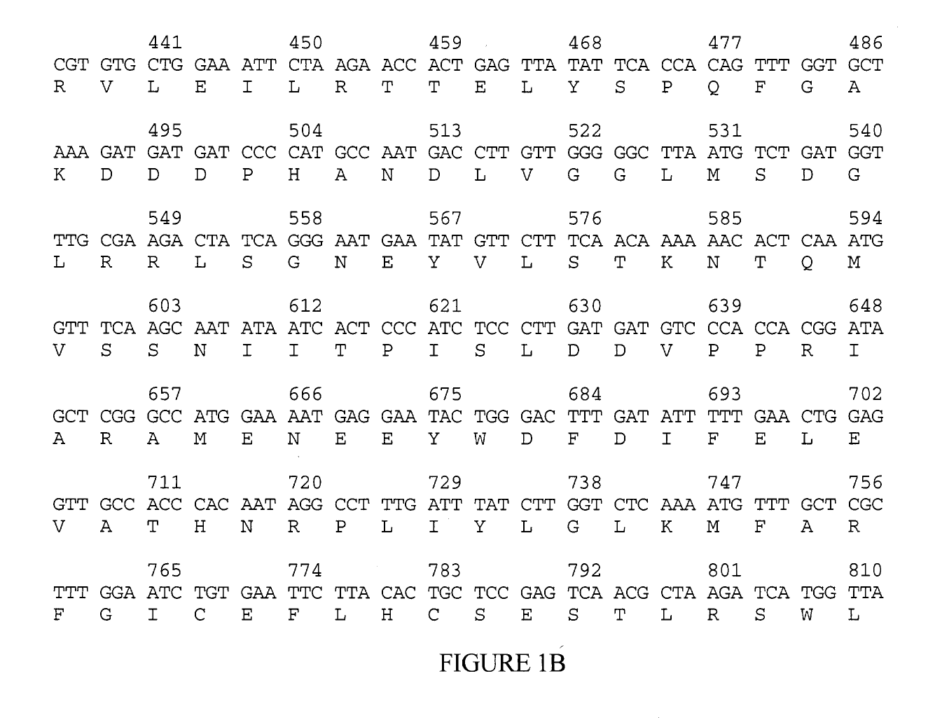

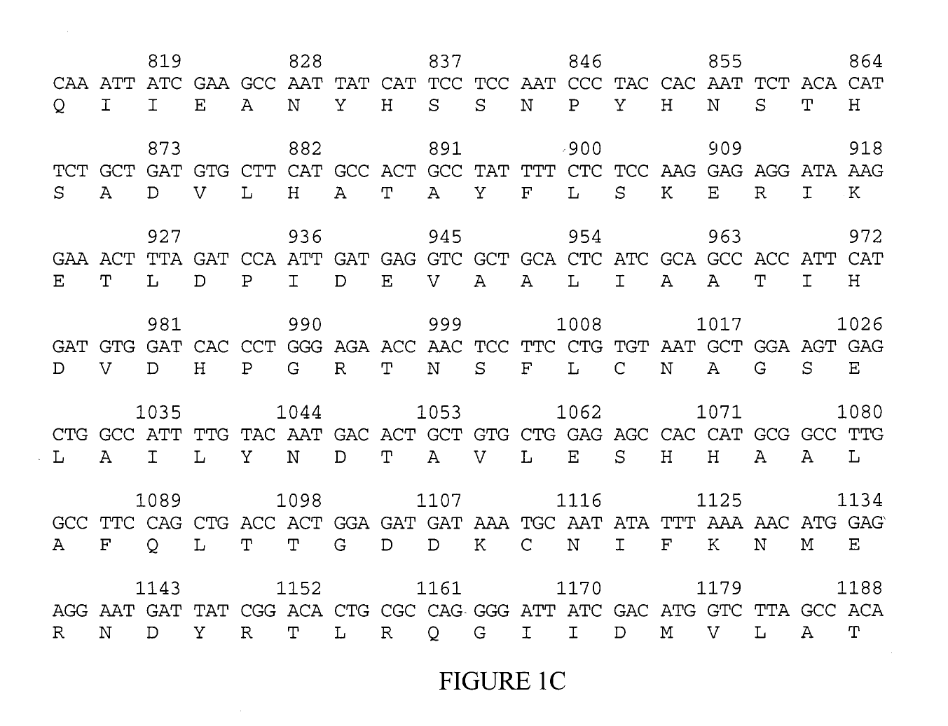

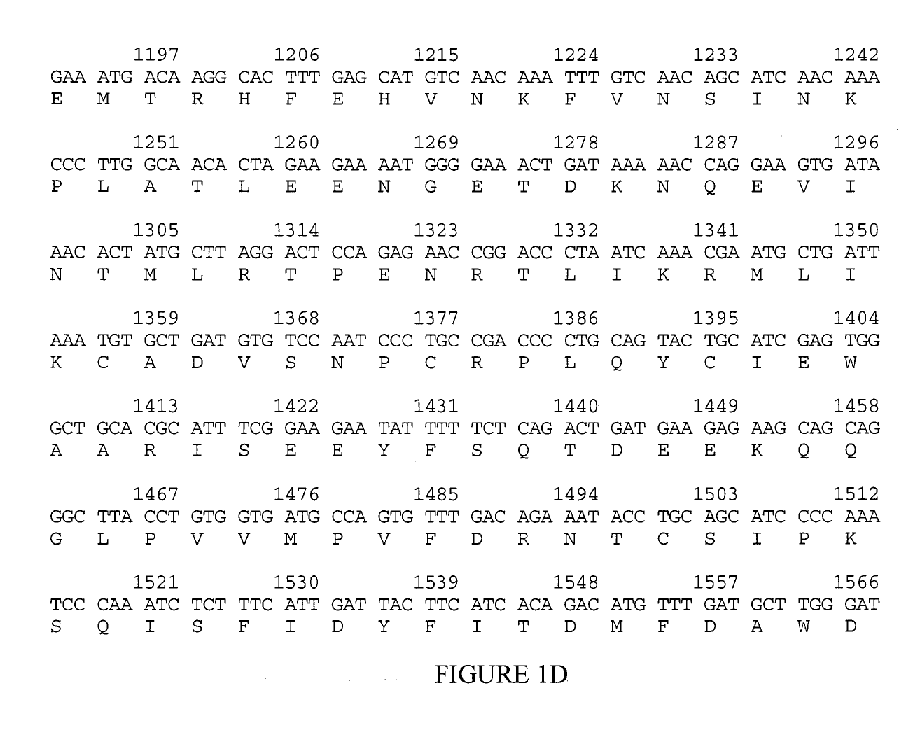

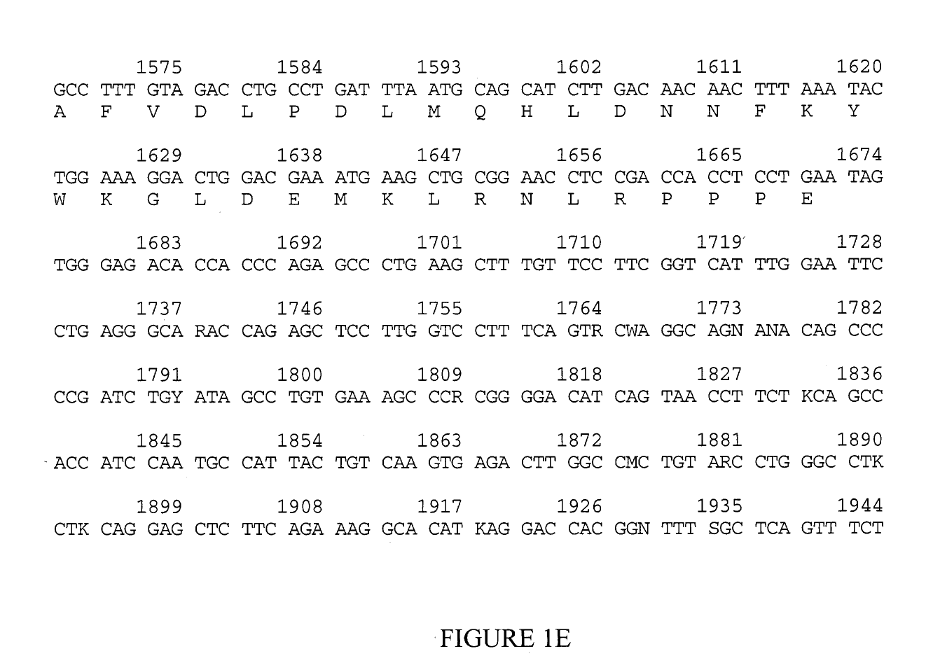

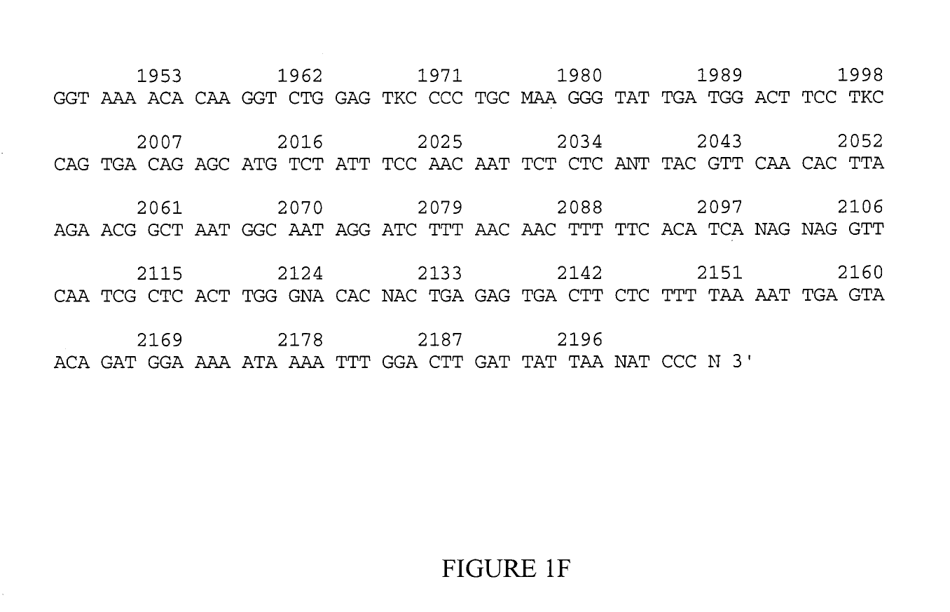

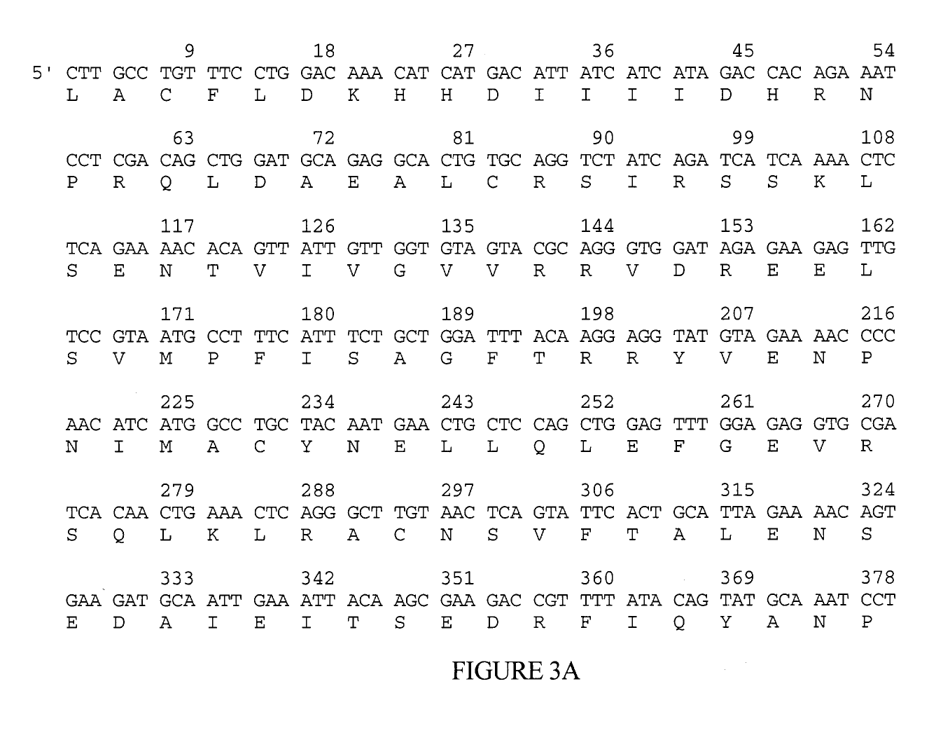

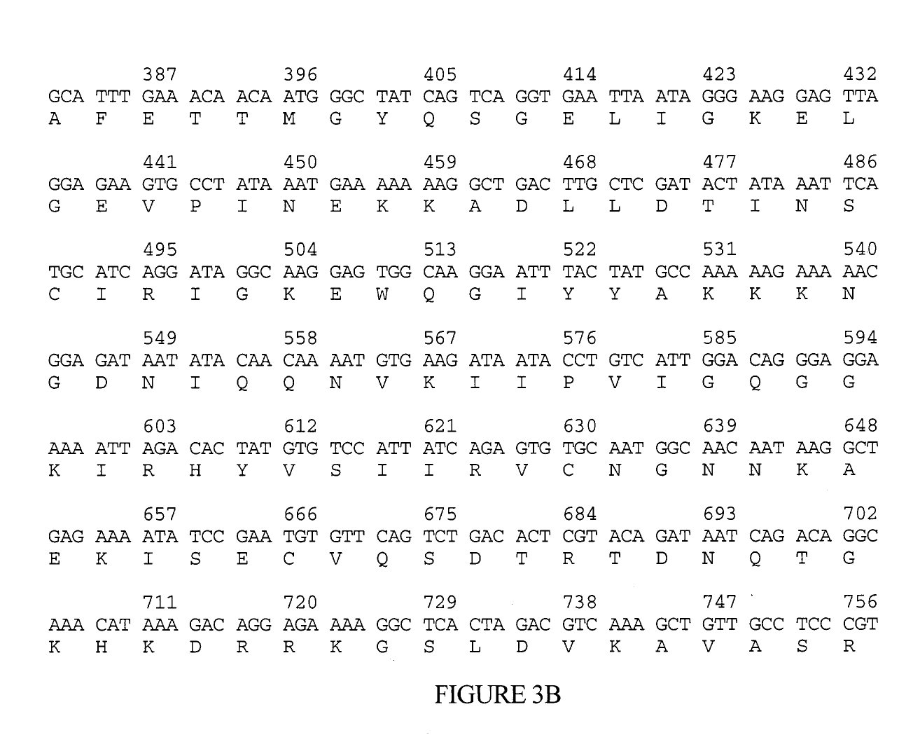

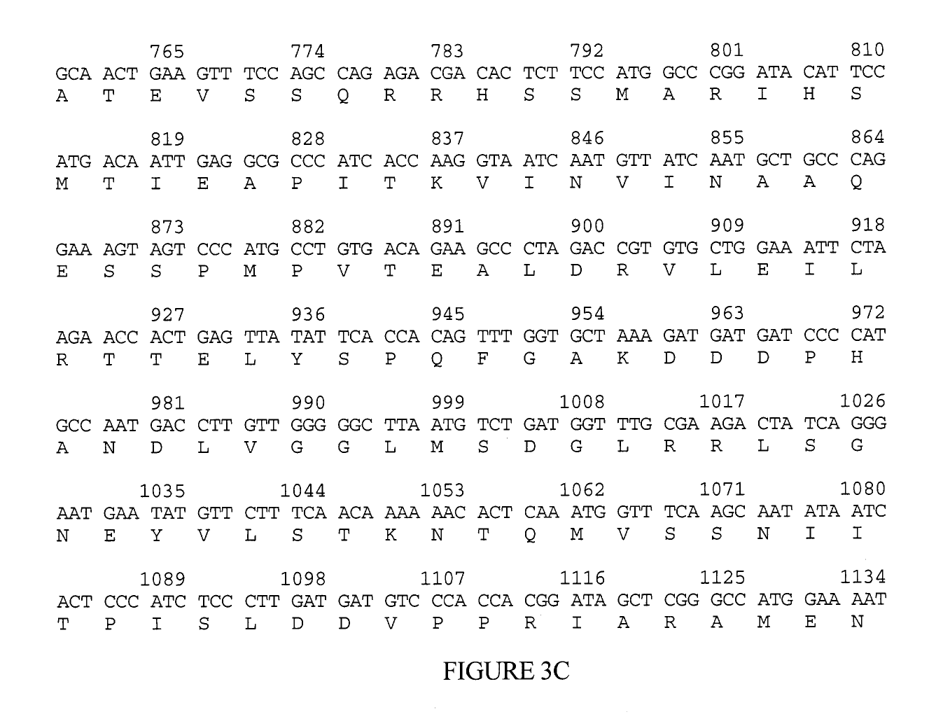

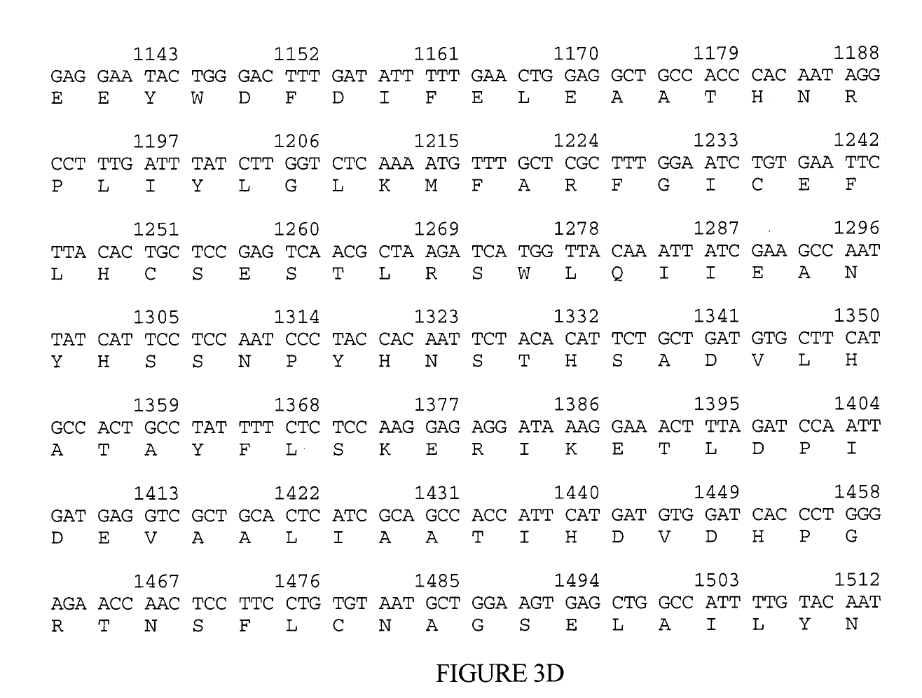

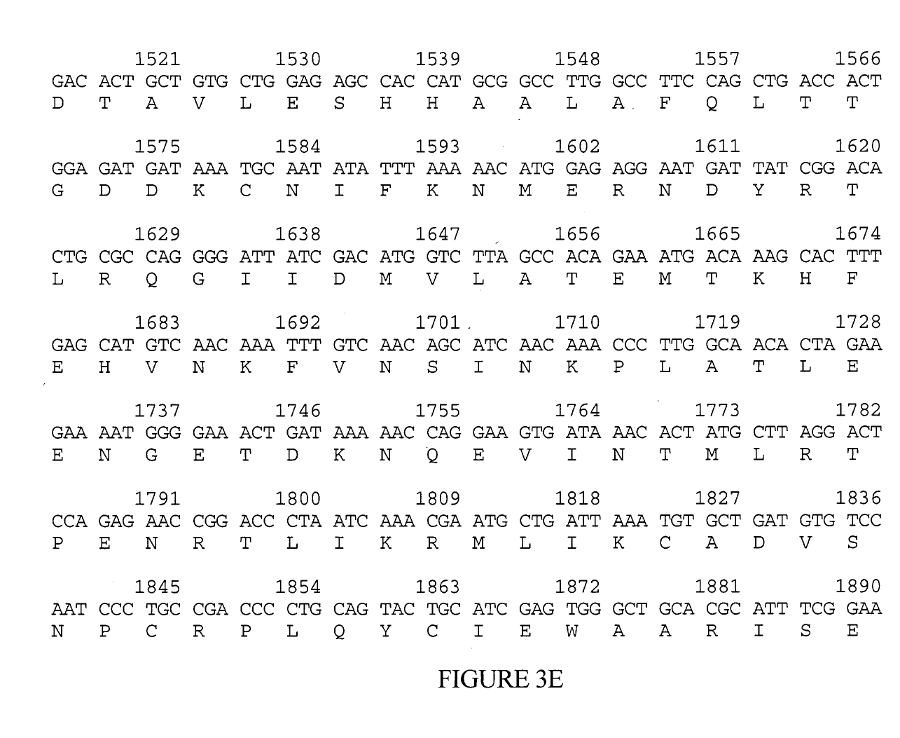

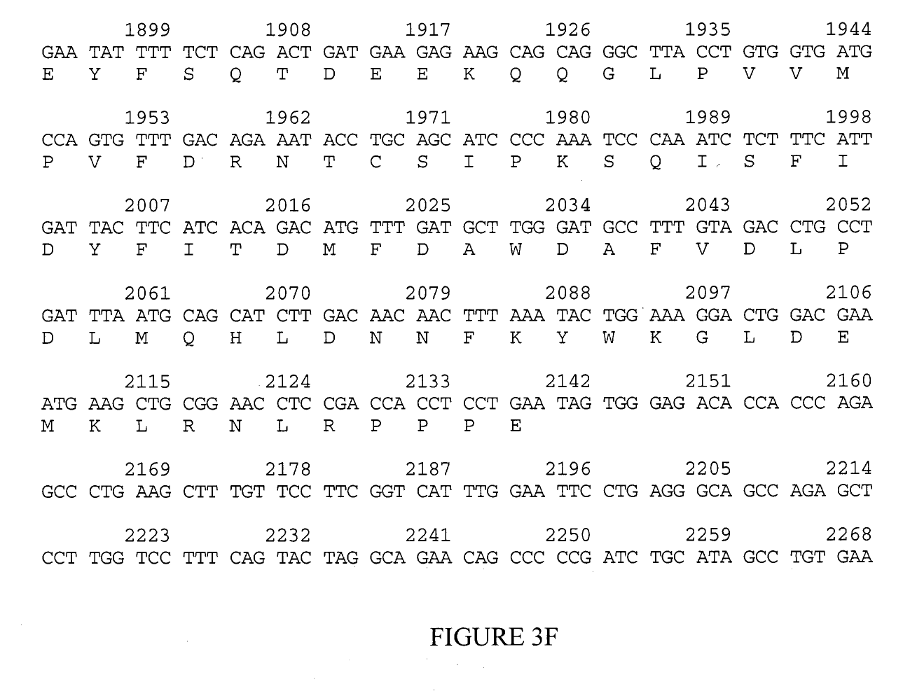

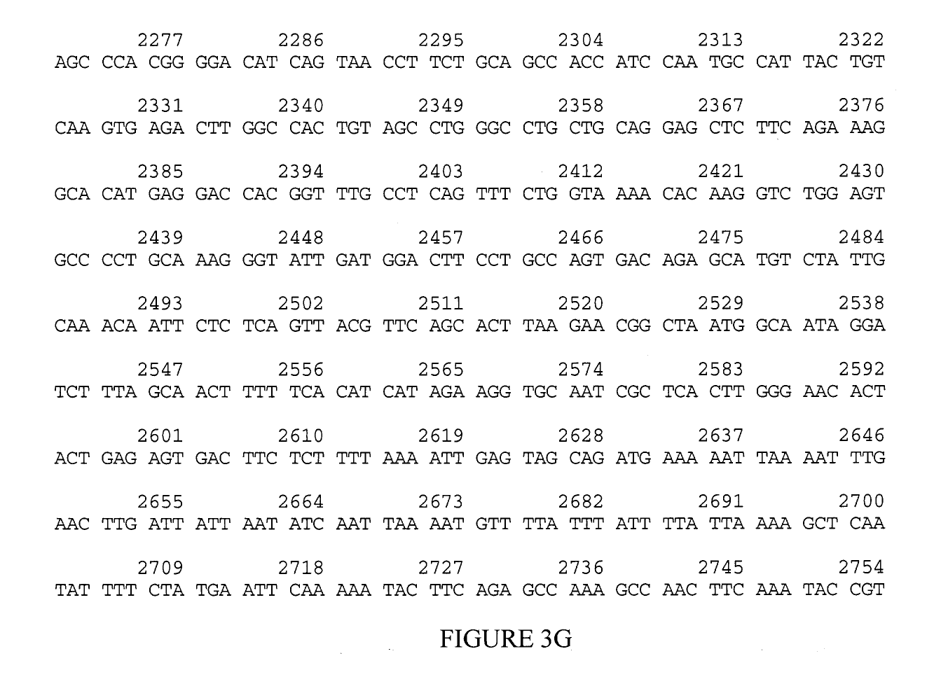

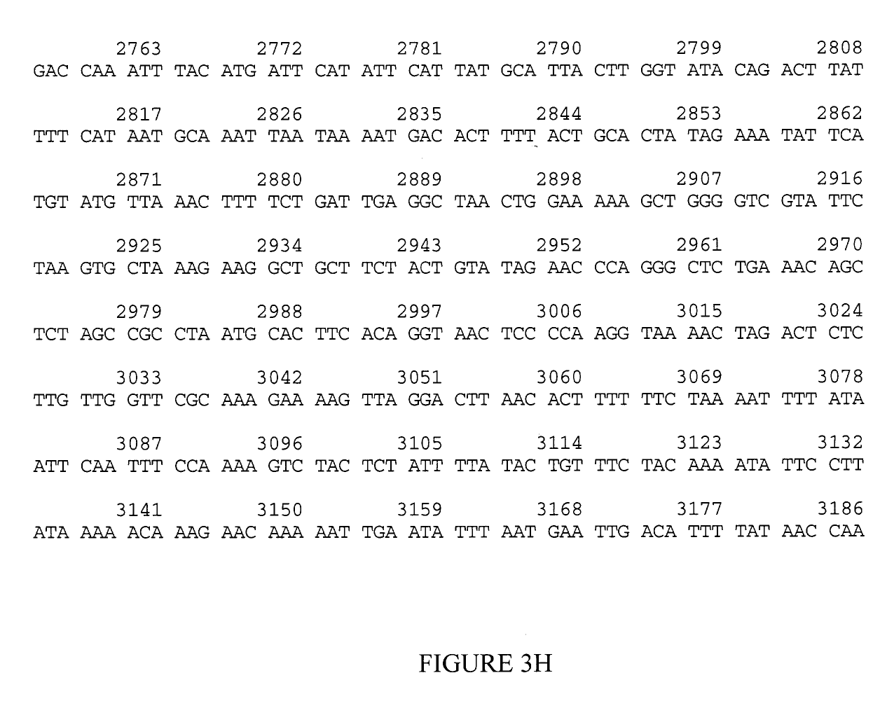

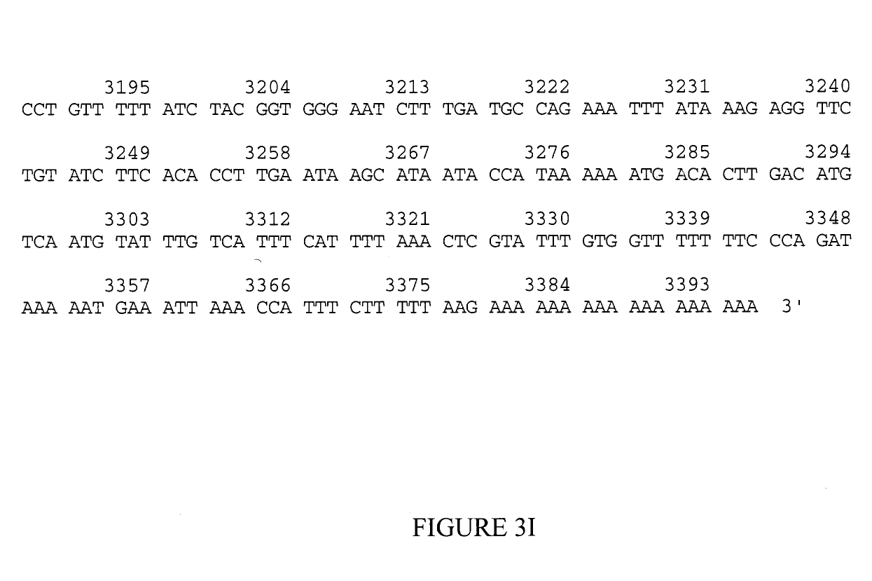

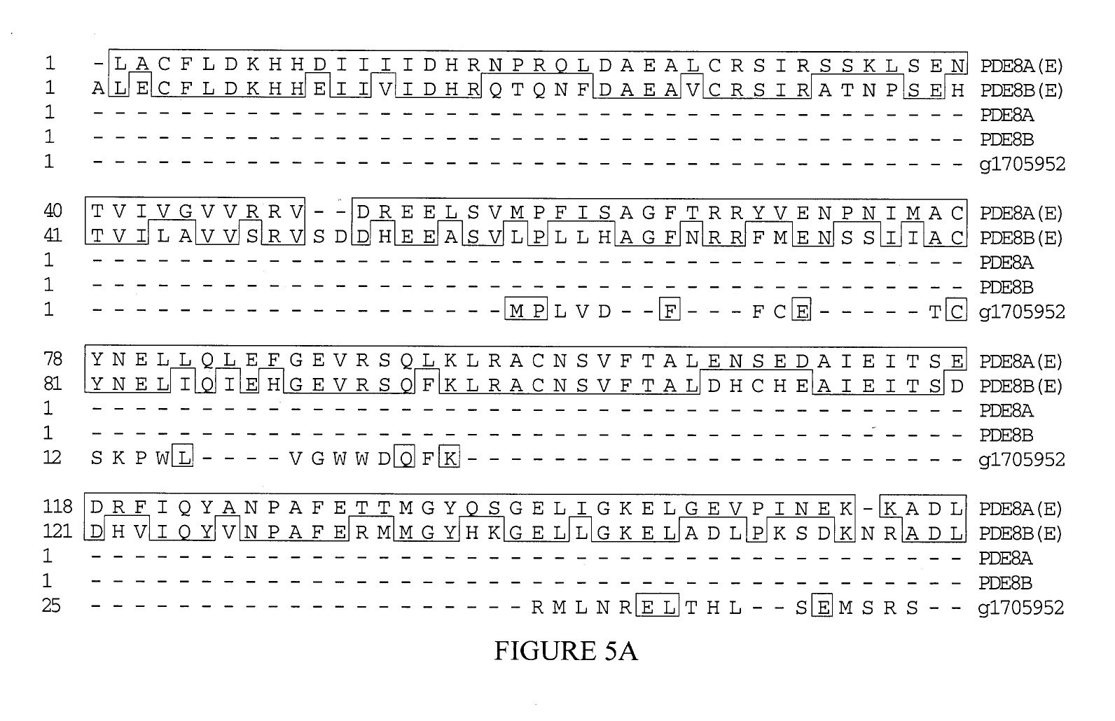

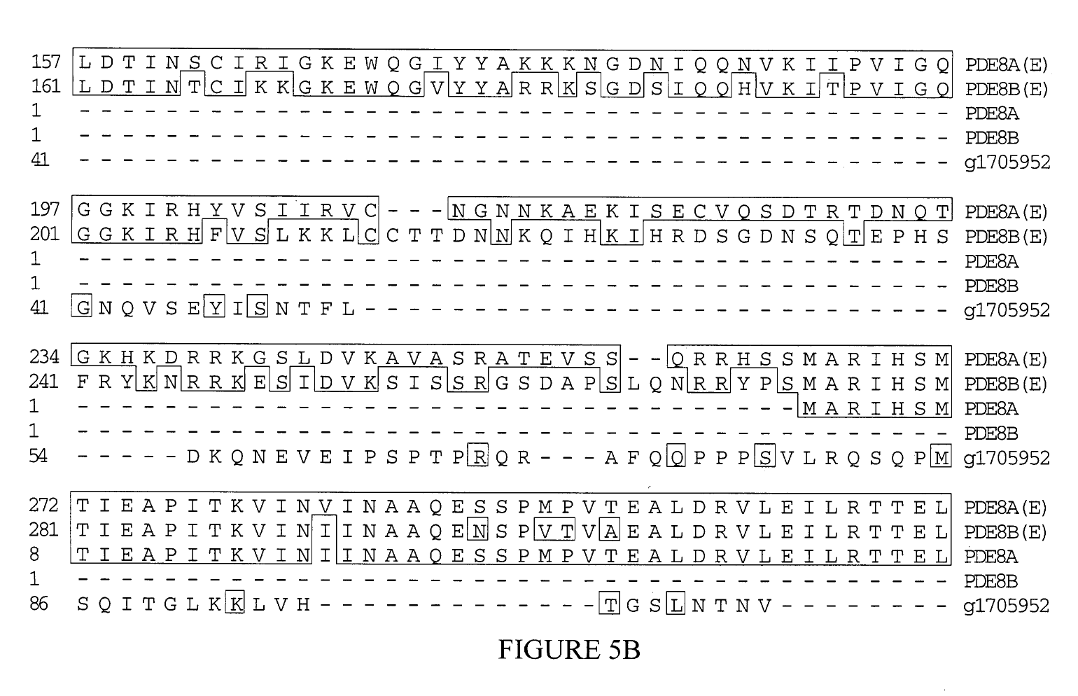

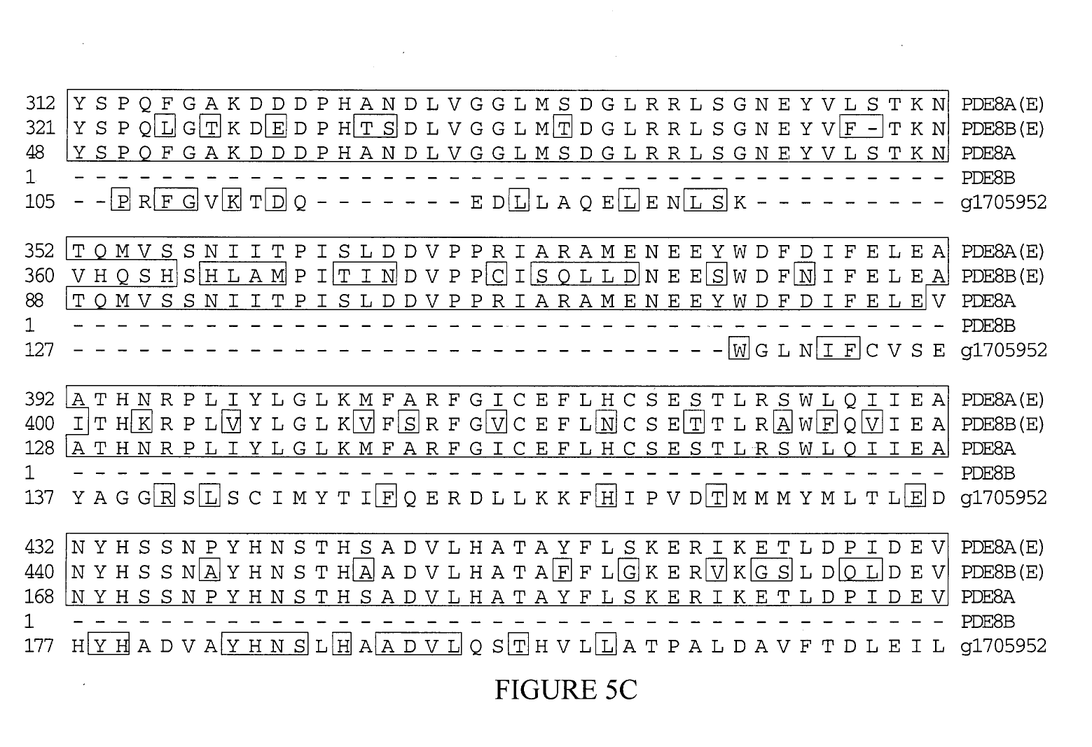

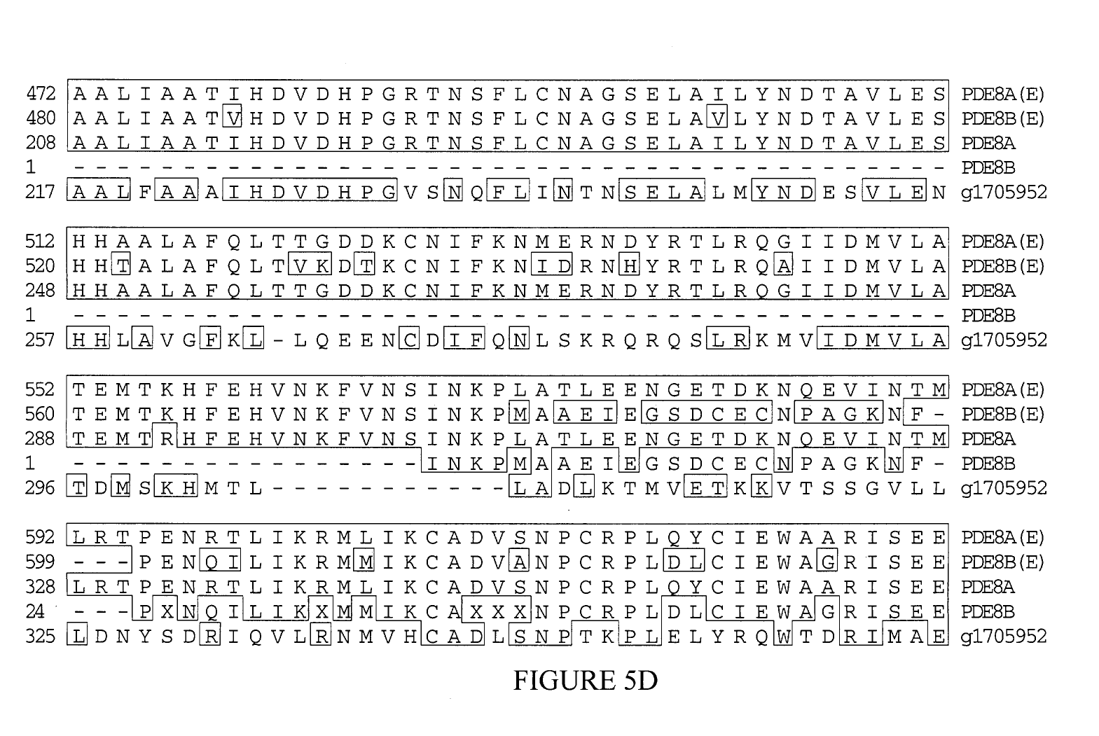

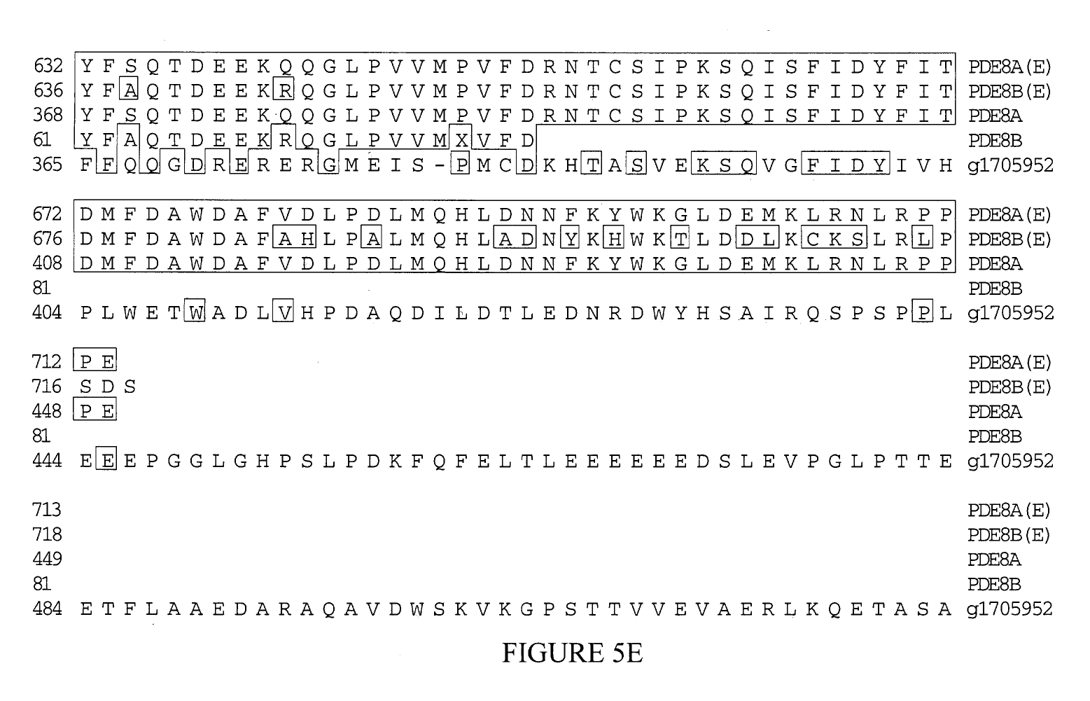

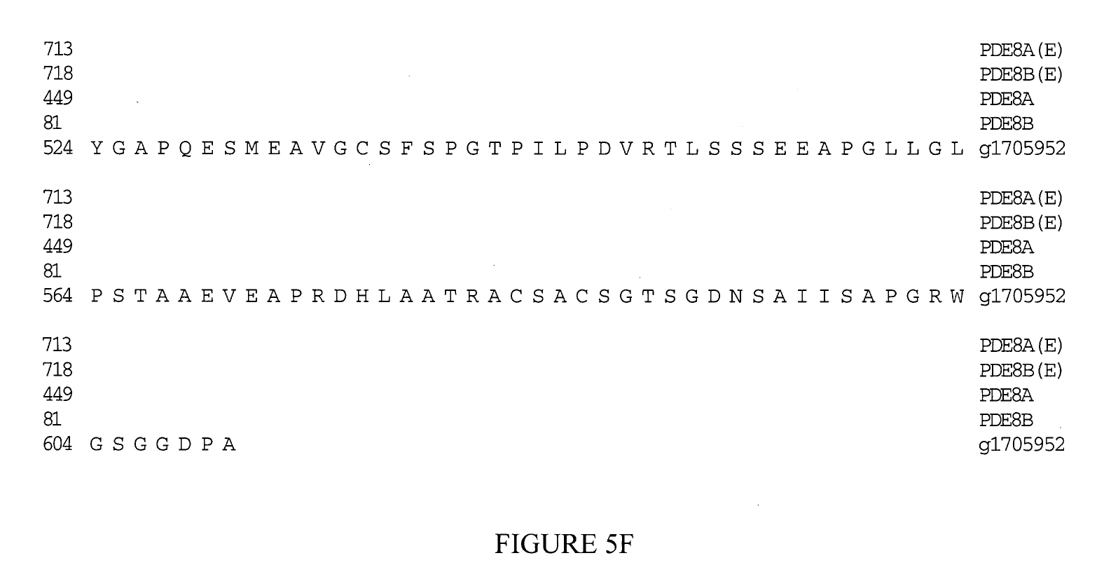

In one embodiment, the invention encompasses proteins comprising the amino acid sequences of SEQ ID NO:1 and SEQ ID NO:7 shown in FIGS. 3A-3I and FIGS. 1A-1F, respectively. PDE8A(E) is 713 amino acids in length and has a consensus signature sequence for cyclic nucleotide PDEs at H₄₈₀DVDHPGRTN of SEQ ID NO:1. This sequence is the first of two potential divalent cation binding sites conserved in PDEs and has the general structure of HXXXH(X₆₋₂₄)E. The first site is H440 - - - H₄₅₀ - - - D₄₆₉, and has D₄₆₉ as a conservative amino acid substitution for E. This substitution is also found in PDE7. The second cation binding site is the sequence H₄₈₀ - - - H₄₈₄ - - - E₅₁₀. PDE8A is 449 amino acids in length and has as consensus signature sequence for cyclic nucleotide PDEs beginning at H₂₁₆. The two divalent cation binding sites begin at residues H₁₇₆ and H₂₁₆. As shown in FIGS. 5A-5F, PDE8A(E) (SEQ ID NO:1) has chemical and structural homology with PDE8A (SEQ ID NO:7), PDE8B(E) (SEQ ID NO:8), PDE8B (SEQ ID NO:16), and rat PDE4A (g1705952; SEQ ID NO:17). In particular, PDE8A (E) shares 70% identity with PDE8B in the C-terminal portion of PDE8A (E) beginning at residue I₅₆₈. The amino acid sequence of PDE8A shares 99% identity with SEQ ID NO:1 beginning at residue M₂₆₅ of SEQ ID NO:1; 78% identity with PDE8B(E), and 22% identity with rat PDE4 . The ˜270 amino acid catalytic domain found in all PDEs extends approximately between residues L₄₁₅ and W₆₉₇ of SEQ ID NO:1; SEQ ID NOs:1 and 7 are approximately 34% identical to rat PDE4A in this region. All five PDEs share the two divalent cation binding sites and the consensus signature sequence, HDXXHXGXXN (SEQ ID NO:26).

The results of laboratory northern analysis are presented in EXAMPLE V and electronic northern analysis using the LIFESEQ database (Incyte Genomics, Palo Alto Calif.) showed the expression of PDE8A in immortalized or cancerous libraries (at least 20%) and libraries involving immune response (at least 80%).

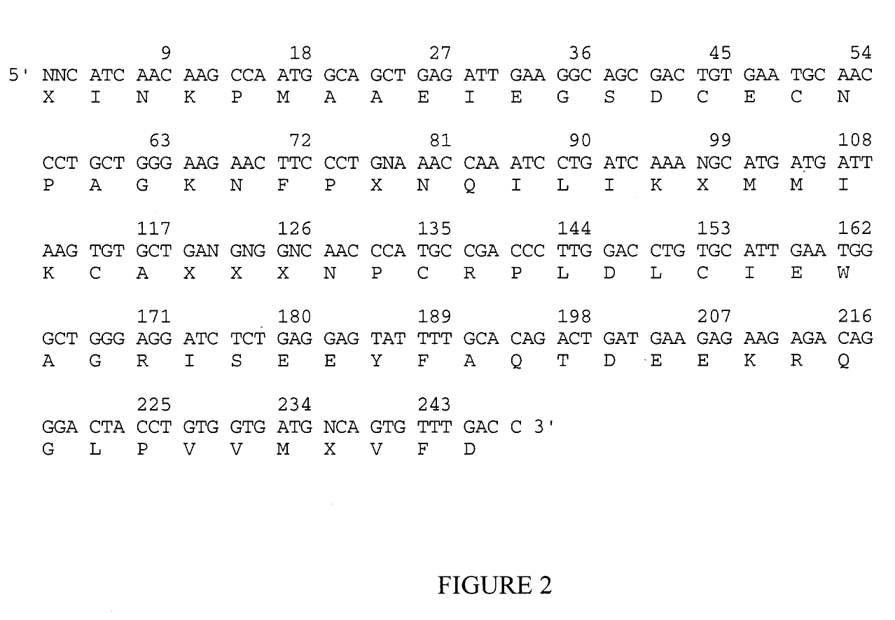

Nucleic acids encoding the PDE8B(E) of the present invention were first identified in Incyte cDNA clone 464655 from the atrial tissue cDNA library (LATRNOT01) using a computer search for amino acid sequence alignments. SEQ ID NO:9 was derived from extension and assembly of Incyte cDNA clones 464655 (LATRNOT01) and 112633 (PITUNOT01). As shown in FIG. 2, SEQ ID NO:10 is a fragment of and has 100% identity to SEQ ID NO:9 with which it aligns from C₁₇₂₆-C₁₉₆₈.

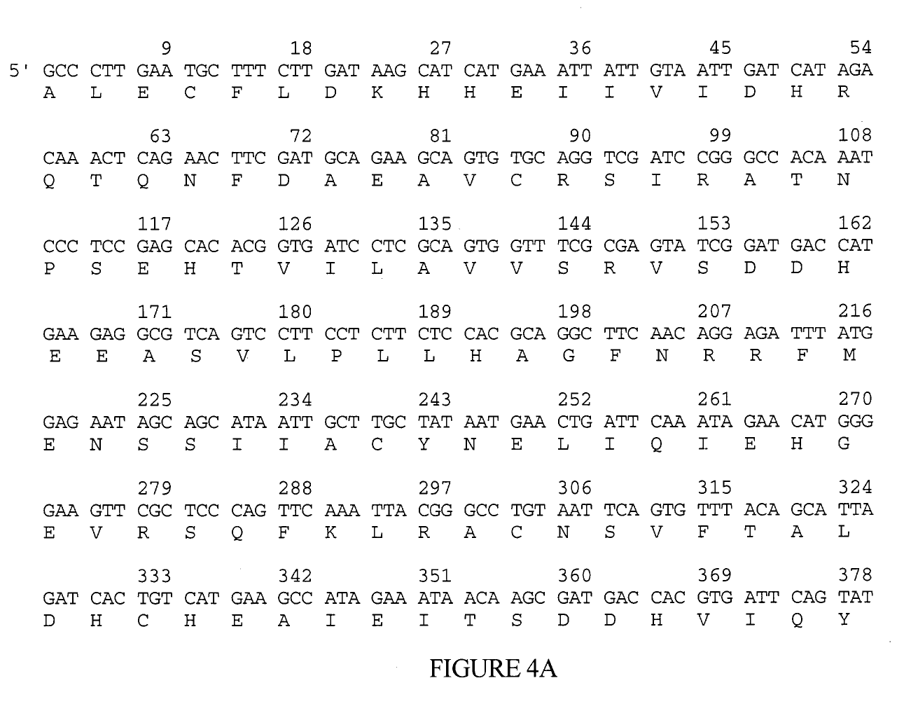

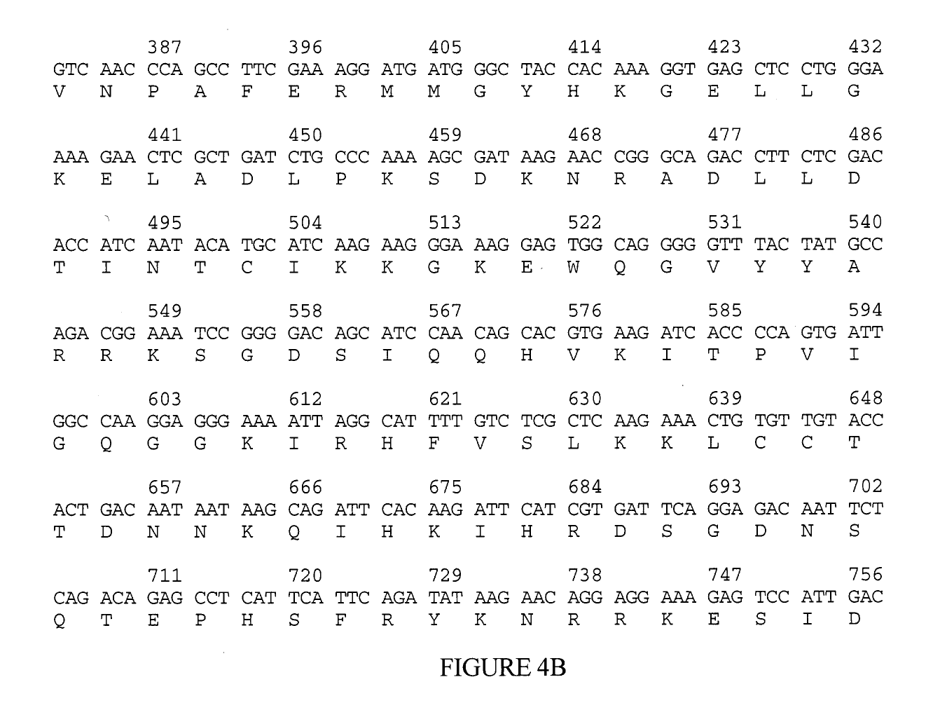

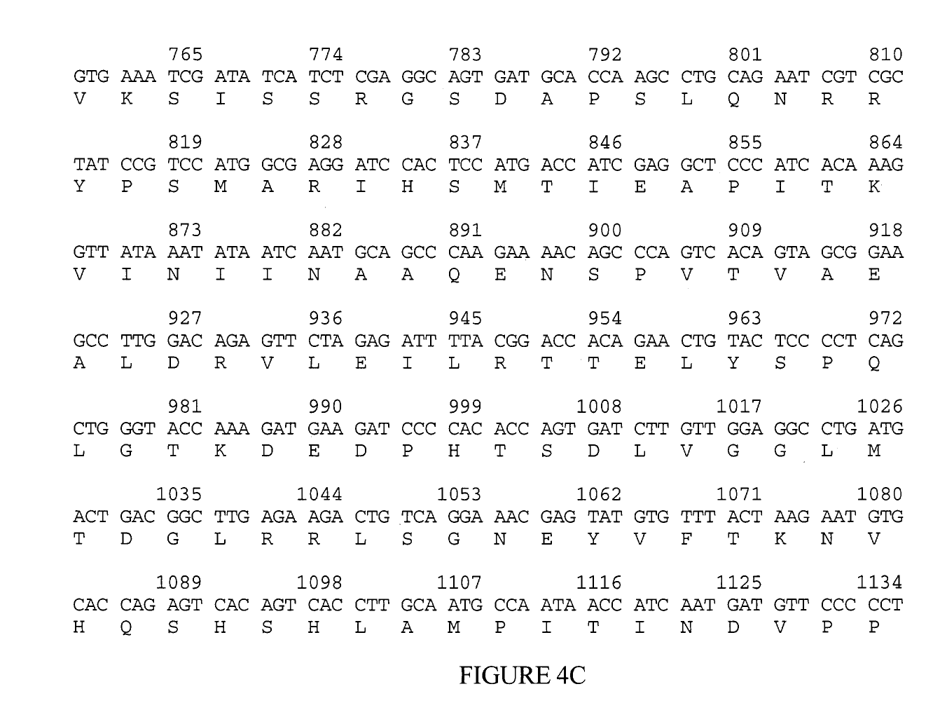

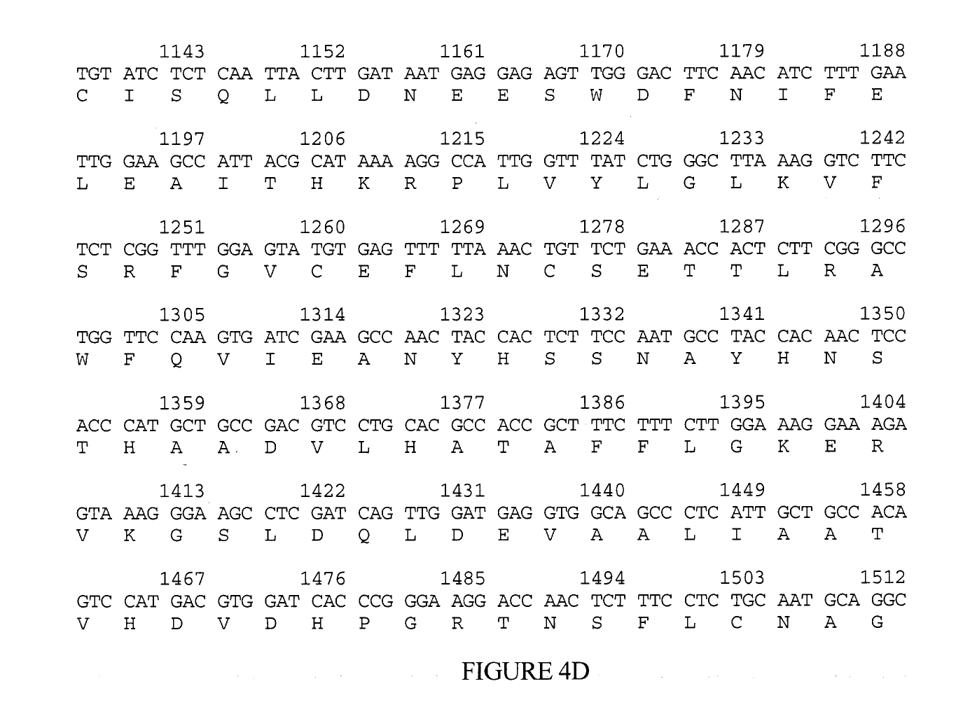

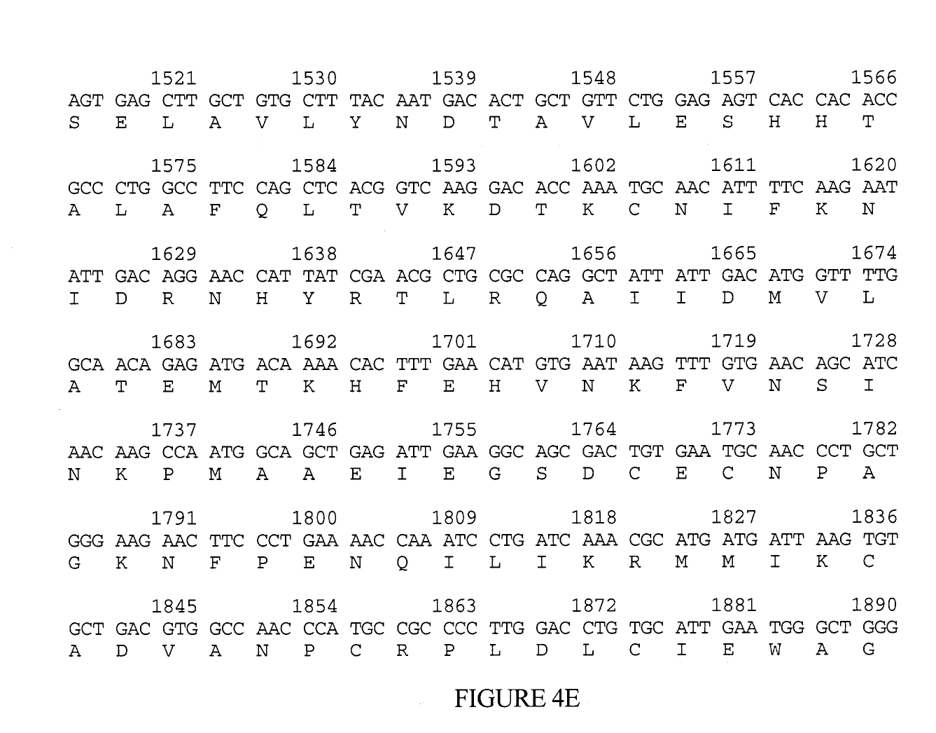

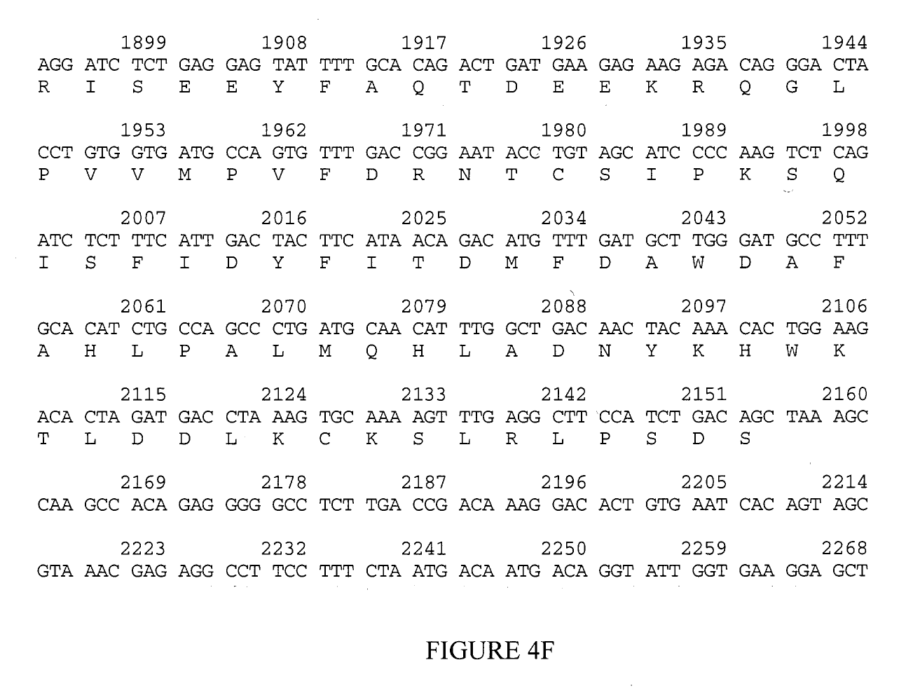

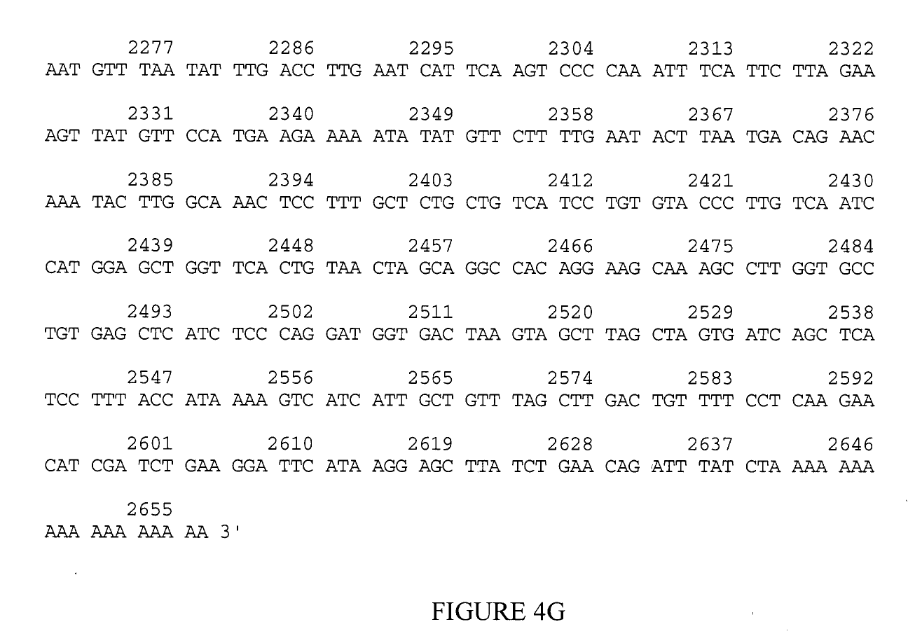

In another embodiment, the invention encompasses a protein comprising the amino acid sequence of SEQ ID NO:8, as shown in FIGS. 4A-4G. PDE8B(E) is 718 amino acids in length and has a consensus cyclic nucleotide PDE signature sequence beginning at H₄₈₈. The two divalent cation binding sites begin at residues H448 and H₄₈₈. As shown in FIGS. 5A-5F, PDE8B(E) has chemical and structural homology with the other PDE8s and with rat PDE4A. In particular, the C-terminal portion of PDE8B(E) is identical (100%) with PDE8B between residues I₅₇₆ and D₆₅₆ of PDE8B(E). PDE8B(E) shares 71% identity with PDE8A(E) and 22% identity with rat PDE4A. PDE8B, as represented by SEQ ID NO:16, is 81 amino acids in length. As shown in FIGS. 5A-5F, PDE8B shares 100% identity with the C-terminal portion of PDE8B(E) beginning at residue I₅₆₈ of SEQ ID NO:8. PDE8B shares 27% identity with rat PDE4A. Electronic northern analysis shows the expression of this sequence in cancer libraries (at least 43%) and in brain and neural libraries (at least 43%).

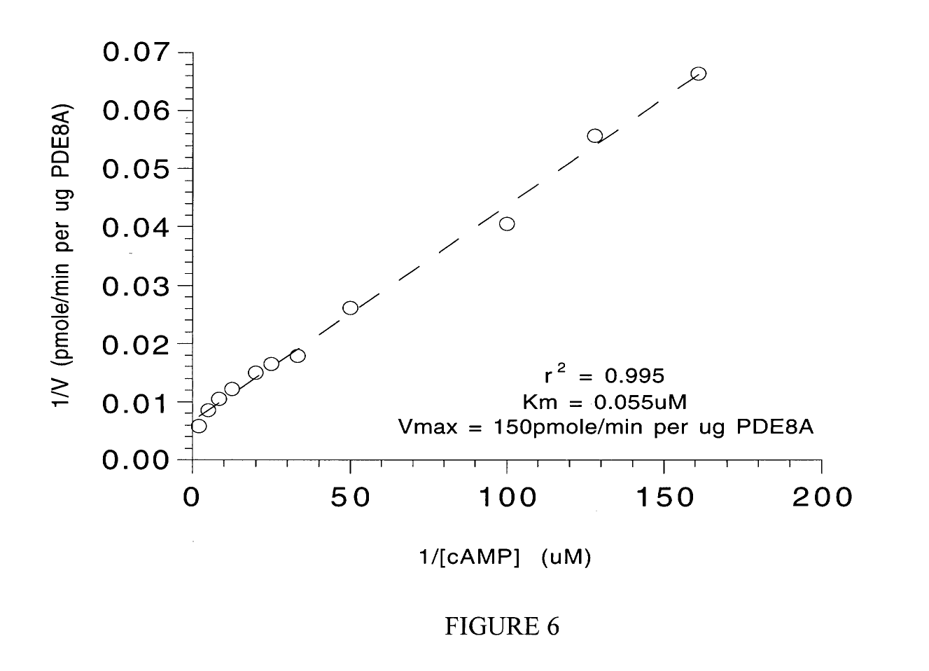

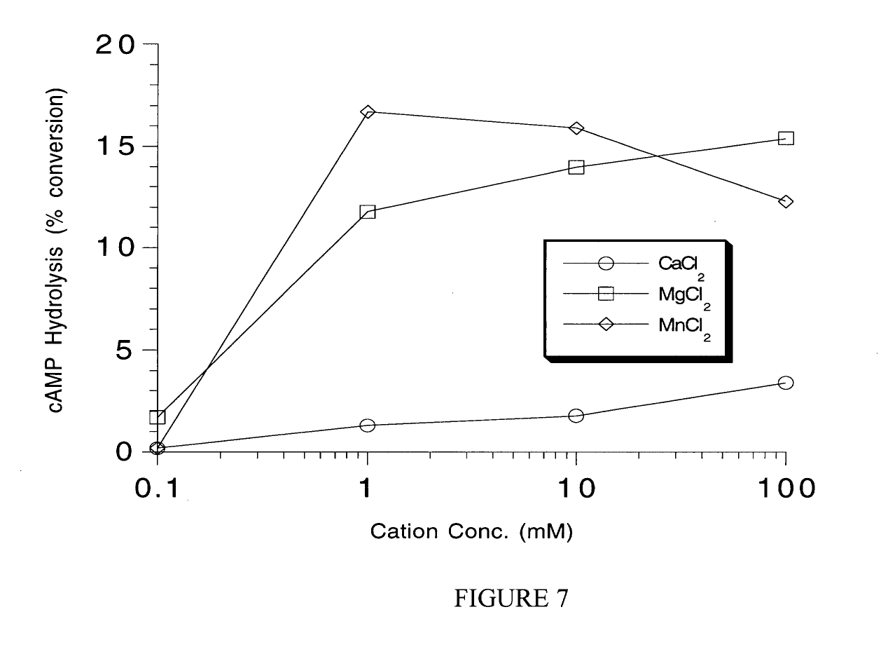

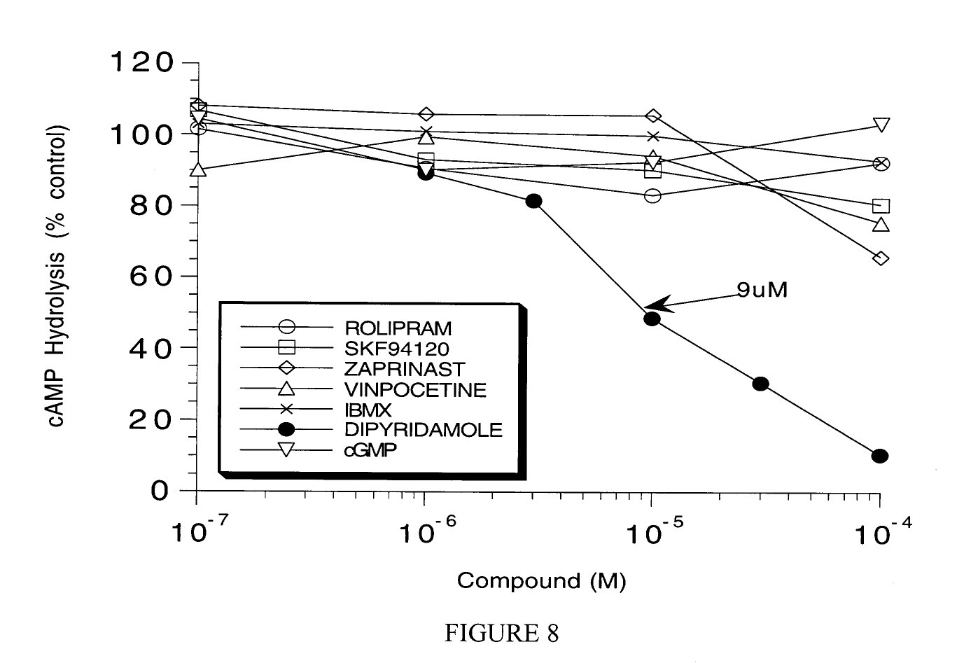

A 1.6 kb region of PDE8A(E) encoding the C-terminal 545 amino acids was cloned into the baculovirus transfer vector pFASTBAC, expressed in sf9 cells, and a cell lysate prepared from these cells for enzyme assays. FIG. 6 shows the kinetics of enzyme activity of recombinant, purified PDE8A(E) with cAMP as a substrate. PDE8A(E) has a very high affinity for cAMP with a Km of 55 nM, and a very low affinity for cGMP (Km=124 mM, data not shown). FIG. 7 shows the dependence of PDE8A(E) on divalent cations for maximal activity with a preference for Mn⁺⁺ or Mg⁺⁺ over Ca⁺⁺. The effects of various known PDE inhibitors on the activity of PDE8A(E) are shown in FIG. 8. PDE8A(E) was not inhibited by up to 100 mM of rolipram, SKF94120 (inhibitor of PDE3), zaprinast (inhibitor of PDE5), vinpocetine (inhibitor of PDE1), of IBMX (non-specific PDE inhibitor). PDE8A(E) was inhibited by dipyridamole (inhibitor of PDE5) with an IC₅₀ of 9 μM. Membrane-based northern analysis shows the expression of this sequence in various tissues, including colon, lung, ovary, pancreas, prostate, small intestine and testis.

The degree of similarity exhibited among the four PDE8 proteins (70% to 100%) is consistent with that shown between isozymes within the same family, while the degree of similarity between the four PDE8 proteins and PDE4 (22% to 29%) is consistent with that shown between isozymes of different families. PDE8A(E) is further distinguished from other known families by its specificity for cAMP and pattern of inhibition by known PDE inhibitors.

The association of PDE8s with neoplastic and immune disorders was based on transcript imaging carried out using the LIFESEQ Gold database (Incyte Genomics) and microarray experiments. Transcripts encoding PDE8s were differentially expressed primarily in prostate and most specifically associated with adenofibromatous hyperplasia and secondarily in pancreas as associated with type 1 diabetes. Transcript images showing the differential expression of PDE8A(E) and PDE8B(E) in prostate and pancreas are shown in EXAMPLE V. Since the transcripts were not expressed in cytologically normal prostate or pancreas, other prostatic or pancreatic disorders, or in metastatic prostate cancer samples or cell lines, these results indicate that an antibody that specifically binds PDE8A(E) and PDE8B(E) is useful as a diagnostic for prostate cancer and type 1 diabetes.

Five microarray experiments were performed to analyze PDE8 expression in tissues and cell lines from primary prostate epithelial cells (PrEC) and prostate carcinomas. These experiments served to compare expression of PDE8 during the prostate tumor progression.

In the table below, the first column identifies the samples used in the experiment, the first sample being labeled with Cy5 and the second, with Cy3; the second column, the descriptions of the samples being compared; and the third column, the occurrence of differential expression (DE) which is, in this instance, defined as, “an increased or upregulated expression as detected by fold change in the amount of transcribed messenger RNA or translated protein in a sample”. The lower limit of significant, detectable differential expression was 1.74-fold at the 95% confidence level (log₂>0.8). The samples are described in EXAMPLE VII.

| Cy5/Cy3 | Descriptions of the Samples Compared* | DE |
|---|---|---|
| PrEC => PZHPV7 | Non-malignant => non-malignant | No |
| PrEC => CAHPV10 | Non-malignant => non-tumorigenic | No |
| PZHPV7 => DU145 (serum free) | Non-malignant => prostate CA, mets^{@} | Yes |
| PC3 => PC3 treated time course | prostate CA, mets | Yes |
| DU145 => DU145 treated time course | prostate CA, mets | Yes |

*Samples and treatments are described below
^{@}mets = metastatic

These results show upregulation of PDE8 expression in prostate cancer cell lines when compared to non-malignant and non-tumorigenic cell lines. The ability to detect significant fold increases in PDE8 transcripts is facilitated in time course experiments with the PC3 and DU145 cell lines. Both cell lines were obtained from metastatic tissue from patients with prostate adenocarcinomas. PC3 cells treated with the transcriptional activators, PMA+ionomycin, showed a dose response over an 8-24 hr treatment period with a maximal response at the 14 hr time point. Similarly, DU145 cells treated with PMA+Ionomycin showed a dose response peaking at the 8 hr time point with a 2.17-fold increase in PDE8 expression (log₂=1.12).

In microarray experiments, PDE8B(E) was differentially expressed in lung cancer. The Human Genome GEM series 1 (HG1) microarray (Incyte Genomics) was used in the experiments summarized in the table below. HG1 contains 9,766 array elements which represent 7,612 annotated clusters and 1,382 unannotated clusters. Log₂ values>1.0 (two-fold) indicate significant differential expression of the mRNA encoding PDE8B(E) in these experiments. Column one of the table shows the log2(Cy5/Cy3) ratio; column two, the description of the Cy3 normal sample; column three, the description of the Cy5 tumor sample; and column four, the donor ID which is described in EXAMPLE VII.

| log2(Cy5/Cy3) | Description of Normal Lung Tissue | Description of Lung Tumor | Donor ID |
|---|---|---|---|
| −1.031943 | mw/Non-Small Cell Lung AdenoCA | Non-Small Cell Lung AdenoCA | 7966 |
| −1.049753 | mw/Squamous Non-Small Cell Lung CA | Squamous Non-Small Cell Lung CA | 7972 |
| −1.070922 | Right Upper Lobe, mw/AdenoCA | Right Upper Lobe, AdenoCA | 7175 |
| −1.228028 | mw/Non-Small Cell Lung CA | Non-Small Cell Lung CA | 7973 |
| −1.398549 | mw/Non-Small Cell Lung AdenoCA | Non-Small Cell Lung AdenoCA | 7965 |

Abbreviations:
mw/ = matched with;
CA = cancer or carcinoma; and
AdenoCA = adenocarcinoma

Mammalian variants of the cDNAs encoding PDE8s were identified using BLAST2 with default parameters and the ZOOSEQ databases (Incyte Genomics). These highly homologous cDNAs have about 90% identity to all or part of the coding region of the human cDNA as shown in the table below. The first column of the table shows the SEQ ID NO: for the human cDNA, the second column, the SEQ ID NO for the homologous cDNAs (SEQ ID_{Var}); the third column, the Incyte ID for the homologous cDNAs (Incyte ID_{Var}); the fourth column, the species; the fifth column, the percent identity to the human cDNA; and the sixth column, the nucleotide alignment of the homologous cDNA to the human cDNA.

| SEQ ID | SEQ ID_{Var} | Incyte ID_{Var} | Species | Identity | Nt_{H} Alignment |
|---|---|---|---|---|---|
| 2 | 4 | 025320_Mf.1 | Monkey | 92% | 2817-2821 |
| 2 | 5 | 052438_Cf.1 | Dog | 90% | 1-593 |
| 2 | 6 | 700310355F6 | Rat | 84% | 1183-1709 |
| 9 | 11 | 003237_Mf.1 | Monkey | 94% | 1402-1646 |
| 9 | 12 | 704105231J1 | Dog | 93% | 660-1335 |
| 9 | 13 | 143773_Mm.1 | Mouse | 90% | 1245-1940 |
| 9 | 14 | 131862_Mm.1 | Mouse | 90% | 1-655 |
| 9 | 15 | 199543_Rn.1 | Rat | 91% | 1576-2335 |

It will be appreciated by those skilled in the art that as a result of the degeneracy of the genetic code, a multitude of cDNAs encoding PDE8s, some bearing minimal similarity to the cDNAs of any known and naturally occurring gene, may be produced. Thus, the invention contemplates each and every possible variation of cDNA that could be made by selecting combinations based on possible codon choices. These combinations are made in accordance with the standard triplet genetic code as applied to the polynucleotide encoding naturally occurring PDE8s, and all such variations are to be considered as being specifically disclosed.

The cDNAs of SEQ ID NOs:2-6 and 9-15 may be used in hybridization, amplification, and screening technologies to identify and distinguish among SEQ ID NO:2 and related molecules in a sample. The mammalian cDNAs may also be used to produce transgenic cell lines or organisms which are model systems for human disorders and upon which the toxicity and efficacy of therapeutic treatments may be tested. Toxicology studies, clinical trials, and subject/patient treatment profiles may be performed and monitored using the cDNAs, proteins, antibodies and molecules and compounds identified using the cDNAs and proteins of the present invention.

Characterization and Use of the Invention

cDNA Libraries

In a particular embodiment disclosed herein, mRNA is isolated from mammalian cells and tissues using methods which are well known to those skilled in the art and used to prepare the cDNA libraries. The Incyte cDNAs were isolated from mammalian cDNA libraries prepared as described in the EXAMPLES. The consensus sequence is present in a single clone insert, or chemically assembled, based on the electronic assembly from sequenced fragments including Incyte cDNAs and extension and/or shotgun sequences. Computer programs, such as PHRAP (P Green, University of Washington, Seattle Wash.) and the AUTOASSEMBLER application (ABI), are used in sequence assembly and are described in EXAMPLE V. After verification of the 5′ and 3′ sequence, at least one representative cDNA which encodes PDE8s was designated a reagent for research and development.

Sequencing

Methods for sequencing nucleic acids are well known in the art and may be used to practice any of the embodiments of the invention. These methods employ enzymes such as the Klenow fragment of DNA polymerase I, SEQUENASE, Taq DNA polymerase and thermostable T7 DNA polymerase (Amersham Biosciences (APB), Piscataway N.J.), or combinations of polymerases and proofreading exonucleases (Invitrogen, Carlsbad Calif.). Sequence preparation is automated with machines such as the MICROLAB 2200 system (Hamilton, Reno Nev.) and the DNA ENGINE thermal cycler (MJ Research, Watertown Mass.) and sequencing, with the PRISM 3700, 377 or 373 DNA sequencing systems (ABI) or the MEGABACE 1000 DNA sequencing system (APB).

The nucleic acid sequences of the cDNAs presented in the Sequence Listing were prepared by such automated methods and may contain occasional sequencing errors and unidentified nucleotides, designated with an N, that reflect state-of-the-art technology at the time the cDNA was sequenced. Vector, linker, and polyA sequences were masked using algorithms and programs based on BLAST, dynamic programming, and dinucleotide nearest neighbor analysis. Ns and SNPs can be verified either by resequencing the cDNA or using algorithms to compare multiple sequences that overlap the area in which the Ns or SNP occur. Both of these techniques are well known to and used by those skilled in the art. The sequences may be analyzed using a variety of algorithms described in Ausubel et al. (1997; Short Protocols in Molecular Biology, John Wiley & Sons, New York N.Y., unit 7.7) and in Meyers (1995; Molecular Biology and Biotechnology, Wiley VCH, New York N.Y., pp. 856-853).

Shotgun sequencing may also be used to complete the sequence of a particular cloned insert of interest. Shotgun strategy involves randomly breaking the original insert into segments of various sizes and cloning these fragments into vectors. The fragments are sequenced and reassembled using overlapping ends until the entire sequence of the original insert is known. Shotgun sequencing methods are well known in the art and use thermostable DNA polymerases, heat-labile DNA polymerases, and primers chosen from representative regions flanking the cDNAs of interest. Incomplete assembled sequences are inspected for identity using various algorithms or programs such as CONSED (Gordon (1998) Genome Res 8:195-202) which are well known in the art. Contaminating sequences, including vector or chimeric sequences, can be removed, and deleted sequences can be restored to complete the assembled, finished sequences.

Extension of a Nucleic Acid

The sequences of the invention may be extended using various PCR-based methods known in the art and detailed in EXAMPLE III.

Hybridization

The cDNA and fragments thereof can be used in hybridization technologies for various purposes. A probe may be designed or derived from unique regions such as the 5′ regulatory region or from a nonconserved region (i.e., 5′ or 3′ of the nucleotides encoding the conserved catalytic domain of the protein) and used in protocols to identify naturally occurring molecules encoding the PDE8s, allelic variants, or related molecules. The probe may be DNA or RNA, may be single-stranded, and should have at least 50% sequence identity to any of the nucleic acid sequences, SEQ ID NOs:2-15. Hybridization probes may be produced using oligolabeling, nick-translation, end-labeling, or PCR amplification in the presence of a reporter molecule. A vector containing the cDNA or a fragment thereof may be used to produce an mRNA probe in vitro by addition of an RNA polymerase and labeled nucleotides. These procedures may be conducted using kits such as those provided by APB.

The stringency of hybridization is determined by G+C content of the probe, salt concentration, and temperature. In particular, stringency can be increased by reducing the concentration of salt or raising the hybridization temperature. Hybridization, although well known in the art, is described in detail in EXAMPLE VII, and reviewed in Ausubel (supra) and Sambrook et al. (1989) Molecular Cloning, A Laboratory Manual, Cold Spring Harbor Press, Plainview N.Y.

Arrays may be prepared and analyzed using methods well known in the art. Oligonucleotides or cDNAs may be used as hybridization probes or targets to monitor the expression level of large numbers of genes simultaneously or to identify genetic variants, mutations, and single nucleotide polymorphisms. Arrays may be used to determine gene function; to understand the genetic basis of a condition, disease, or disorder; to diagnose a condition, disease, or disorder; and to develop and monitor the activities of therapeutic agents. (See, e.g., U.S. Pat. No. 5,474,796; Schena et al. (1996) Proc Natl Acad Sci 93:10614-10619; Heller et al. (1997) Proc Natl Acad Sci 94:2150-2155; U.S. Pat. No. 5,605,662.)

Hybridization probes are also useful in mapping the naturally occurring genomic sequence. The probes may be hybridized to a particular chromosome, a specific region of a chromosome, or an artificial chromosome construction. Such constructions include human artificial chromosomes, yeast artificial chromosomes, bacterial artificial chromosomes, bacterial P1 constructions, or the cDNAs of libraries made from single chromosomes.

QPCR

QPCR is a method for quantifying a nucleic acid molecule based on detection of a fluorescent signal produced during PCR amplification (Gibson et al. (1996) Genome Res 6:995-1001; Heid et al. (1996) Genome Res 6:986-994). Amplification is carried out on machines such as the PRISM 7700 detection system (ABI) which consists of a 96-well thermal cycler connected to a laser and charge-coupled device (CCD) optics system. To perform QPCR, a PCR reaction is carried out in the presence of a doubly labeled probe. The probe, which is designed to anneal between the standard forward and reverse PCR primers, is labeled at the 5′ end by a flourogenic reporter dye such as 6-carboxyfluorescein (6-FAM) and at the 3′ end by a quencher molecule such as 6-carboxy-tetramethyl-rhodamine (TAMRA). As long as the probe is intact, the 3′ quencher extinguishes fluorescence by the 5′ reporter. However, during each primer extension cycle, the annealed probe is degraded as a result of the intrinsic 5′ to 3′ nuclease activity of Taq polymerase (Holland et al. (1991) Proc Natl Acad Sci 88:7276-7280). This degradation separates the reporter from the quencher, and fluorescence is detected every few seconds by the CCD. The higher the starting copy number of the nucleic acid, the sooner an increase in fluorescence is observed. A cycle threshold (C_{T}) value, representing the cycle number at which the PCR product crosses a fixed threshold of detection is determined by the instrument software. The C_{T} is inversely proportional to the copy number of the template and can therefore be used to calculate either the relative or absolute initial concentration of the nucleic acid molecule in the sample. The relative concentration of two different molecules can be calculated by determining their respective C_{T} values (comparative C_{T} method). Alternatively, the absolute concentration of the nucleic acid molecule can be calculated by constructing a standard curve using a housekeeping molecule of known concentration. The process of calculating C_{T} values, preparing a standard curve, and determining starting copy number is performed using SEQUENCE DETECTOR 1.7 software (ABI).

Expression

Any one of a multitude of cDNAs encoding PDE8s may be cloned into a vector and used to express the protein, or portions thereof, in host cells. The nucleic acid sequence can be engineered by such methods as DNA shuffling (U.S. Pat. No. 5,830,721) and site-directed mutagenesis to create new restriction sites, alter glycosylation patterns, change codon preference to increase expression in a particular host, produce splice variants, extend half-life, and the like. The expression vector may contain transcriptional and translational control elements (promoters, enhancers, specific initiation signals, and polyadenylated 3′ sequence) from various sources which have been selected for their efficiency in a particular host. The vector, cDNA, and regulatory elements are combined using in vitro recombinant DNA techniques, synthetic techniques, and/or in vivo genetic recombination techniques well known in the art and described in Sambrook (supra, ch. 4, 8, 16 and 17).

A variety of host systems may be transformed with an expression vector. These include, but are not limited to, bacteria transformed with recombinant bacteriophage, plasmid, or cosmid DNA expression vectors; yeast transformed with yeast expression vectors; insect cell systems transformed with baculovirus expression vectors or plants or their cultured cells transformed with expression vectors containing viral and/or bacterial elements (Ausubel supra, unit 16). In mammalian cell systems, an adenovirus transcriptional/translational complex may be utilized. After sequences are ligated into the E1 or E3 region of the viral genome, the infective virus is used to transform and express the protein in host cells. The Rous sarcoma virus enhancer or SV40 or EBV-based vectors may also be used for high-level protein expression.

Routine cloning, subcloning, and propagation of nucleic acid sequences can be achieved using the multifunctional pBLUESCRIPT vector (Stratagene, La Jolla Calif.) or pSPORT1 plasmid (Invitrogen). Introduction of a nucleic acid sequence into the multiple cloning site of these vectors disrupts the lacZ gene and allows colorimetric screening for transformed bacteria. In addition, these vectors may be useful for in vitro transcription, dideoxy sequencing, single strand rescue with helper phage, and creation of nested deletions in the cloned sequence.

For long term production of recombinant proteins, the vector can be stably transformed into cell lines along with a selectable or visible marker gene on the same or on a separate vector. After transformation, cells are allowed to grow for about 1 to 2 days in enriched media and then are transferred to selective media. Selectable markers, antimetabolite, antibiotic, or herbicide resistance genes, confer resistance to the relevant selective agent and allow growth and recovery of cells which successfully express the introduced sequences. Resistant clones identified either by survival on selective media or by the expression of visible markers may be propagated using culture techniques. Visible markers are also used to estimate the amount of protein expressed by the introduced genes. Verification that the host cell contains the desired cDNA is based on DNA-DNA or DNA-RNA hybridizations or PCR amplification.

The host cell may be chosen for its ability to modify a recombinant protein in a desired fashion. Such modifications include acetylation, carboxylation, glycosylation, phosphorylation, lipidation, acylation and the like. Post-translational processing which cleaves a “prepro” form may also be used to specify protein targeting, folding, and/or activity. Different host cells which have specific cellular machinery and characteristic mechanisms for post-translational activities may be chosen to ensure the correct modification and processing of the recombinant protein.

Recovery of Proteins from Cell Culture

Heterologous moieties engineered into a vector for ease of purification include glutathione S-transferase (GST), 6xHis, FLAG, MYC, and the like. GST and 6-His are purified using affinity matrices such as immobilized glutathione and metal-chelate resins, respectively. FLAG and MYC are purified using monoclonal and polyclonal antibodies. For ease of separation following purification, a sequence encoding a proteolytic cleavage site may be part of the vector located between the protein and the heterologous moiety. Methods for recombinant protein expression and purification are discussed in Ausubel (supra, unit 16).

Protein Identification

Several techniques have been developed which permit rapid identification of proteins using high performance liquid chromatography (HPLC) and mass spectrometry (MS). Beginning with a sample containing proteins, the method is: 1) proteins are separated using electrophoresis, 2) selected proteins are excised from the gel and digested with a protease to produce a set of peptides; and 3) the peptides are subjected to HPLC to elucidate and quantify their amino acids or to MS to derive peptide ion mass and spectral pattern information. The MS information is used to identify the protein by comparing it with information in a protein database (Shevenko et al. (1996) Proc Natl Acad Sci 93:14440-14445).

Proteins are separated by employing isoelectric focusing (IEF) in the first dimension followed by SDS-PAGE in the second dimension. For IEF, an immobilized pH gradient strip is useful to increase reproducibility and resolution of the separation. Alternative techniques may be used to improve resolution of very basic, hydrophobic, or high molecular weight proteins. The separated proteins are detected using a stain or dye such as silver stain, Coomassie blue, or spyro red (Molecular Probes, Eugene Oreg.) that is compatible with MS. Gels may be blotted onto a PVDF membrane for western analysis and optically scanned using a STORM scanner (APB) to produce a computer-readable output which is analyzed by pattern recognition software such as MELANIE (GeneBio, Geneva, Switzerland). The software annotates individual spots by assigning a unique identifier and calculating their respective x,y coordinates, molecular masses, isoelectric points, and signal intensity. Individual spots of interest, such as those representing differentially expressed proteins, are excised and proteolytically digested with a site-specific protease such as trypsin or chymotrypsin, singly or in combination, to generate a set of small peptides, preferably in the range of 1-2 kDa. Prior to digestion, samples may be treated with reducing and alkylating agents, and following digestion, the peptides are then separated by liquid chromatography or capillary electrophoresis and analyzed using MS.

MS converts components of a sample into gaseous ions, separates the ions based on their mass-to-charge ratio, and determines relative abundance. For peptide mass fingerprinting analysis, a MALDI-TOF (Matrix Assisted Laser Desorption/Ionization-Time of Flight), ESI (Electrospray Ionization), and TOF-TOF (Time of Flight/Time of Flight) machines are used to determine a set of highly accurate peptide masses. Using analytical programs, such as TURBOSEQUEST software (Finnigan, San Jose Calif.), the MS data is compared against a database of theoretical MS data derived from known or predicted proteins. A minimum match of three peptide masses is used for reliable protein identification. If additional information is needed for identification, Tandem-MS may be used to derive information about individual peptides. In tandem-MS, a first stage of MS is performed to determine individual peptide masses. Then selected peptide ions are subjected to fragmentation using a technique such as collision induced dissociation (CID) to produce an ion series. The resulting fragmentation ions are analyzed in a second round of MS, and their spectral pattern may be used to determine a short stretch of amino acid sequence (Dancik et al. (1999) J Comput Biol 6:327-342).

Assuming the protein is represented in the database, a combination of peptide mass and fragmentation data, together with the calculated MW and pI of the protein, will usually yield an unambiguous identification. If no match is found, protein sequence can be obtained using direct chemical sequencing procedures well known in the art (cf. Creighton (1984) Proteins, Structures and Molecular Properties, W H Freeman, New York N.Y.).

Chemical Synthesis of Peptides

Proteins or portions thereof may be produced not only by recombinant methods, but also by using chemical methods well known in the art. Solid phase peptide synthesis may be carried out in a batchwise or continuous flow process which sequentially adds α-amino- and side chain-protected amino acid residues to an insoluble polymeric support via a linker group. A linker group such as methylamine-derivatized polyethylene glycol is attached to poly(styrene-co-divinylbenzene) to form the support resin. The amino acid residues are N-α-protected by acid labile Boc (t-butyloxycarbonyl) or base-labile Fmoc (9-fluorenylmethoxycarbonyl). The carboxyl group of the protected amino acid is coupled to the amine of the linker group to anchor the residue to the solid phase support resin. Trifluoroacetic acid or piperidine are used to remove the protecting group in the case of Boc or Fmoc, respectively. Each additional amino acid is added to the anchored residue using a coupling agent or pre-activated amino acid derivative, and the resin is washed. The full length peptide is synthesized by sequential deprotection, coupling of derivitized amino acids, and washing with dichloromethane and/or N,N-dimethylformamide. The peptide is cleaved between the peptide carboxy terminus and the linker group to yield a peptide acid or amide. (Novabiochem 1997/98 Catalog and Peptide Synthesis Handbook, San Diego Calif. pp. S1-S20). Automated synthesis may also be carried out on machines such as the 431A peptide synthesizer (ABI). A protein or portion thereof may be purified by preparative high performance liquid chromatography and its composition confirmed by amino acid analysis or by sequencing (Creighton, supra)

Preparation and Screening of Antibodies

Various hosts including mice, rats, rabbits, goats, llamas, camels, and human cell lines may be immunized by injection with an antigenic determinant. Adjuvants such as Freund's, mineral gels, and surface active substances such as lysolecithin, pluronic polyols, polyanions, peptides, oil emulsions, KLH, and dinitrophenol may be used to increase immunological response. In humans, BCG (bacilli Calmette-Guerin) and Corynebacterium parvum increase response. The antigenic determinant may be an oligopeptide, peptide, or protein. When the amount of antigenic determinant allows immunization to be repeated, specific polyclonal antibody with high affinity can be obtained (Klinman and Press (1975) Transplant Rev 24:41-83). Oligopepetides which may contain between about five and about fifteen amino acids identical to a portion of the endogenous protein may be fused with proteins such as KLH in order to produce antibodies to the chimeric molecule.

Monoclonal antibodies may be prepared using any technique which provides for the production of antibodies by continuous cell lines in culture. These include the hybridoma technique, the human B-cell hybridoma technique, and the EBV-hybridoma technique (Kohler et al. (1975) Nature 256:495-497; Kozbor et al. (1985) J Immunol Methods 81:31-42; Cote et al. (1983) Proc Natl Acad Sci 80:2026-2030; and Cole et al. (1984) Mol Cell Biol 62:109-120).

Chimeric antibodies may be produced by techniques such as splicing of mouse antibody genes to human antibody genes to obtain a molecule with appropriate antigen specificity and biological activity (Morrison et al. (1984) Proc Natl Acad Sci 81:6851-6855; Neuberger et al. (1984) Nature 312:604-608; and Takeda et al. (1985) Nature 314:452-454). Alternatively, techniques described for antibody production may be adapted, using methods known in the art, to produce specific, single chain antibodies. Antibodies with related specificity, but of distinct idiotypic composition, may be generated by chain shuffling from random combinatorial immunoglobulin libraries (Burton (1991) Proc Natl Acad Sci 88:10134-10137). Antibody fragments which contain specific binding sites for an antigenic determinant may also be produced. For example, such fragments include, but are not limited to, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse et al. (1989) Science 246:1275-1281).

Antibodies may also be produced by inducing production in the lymphocyte population or by screening immunoglobulin libraries or panels of highly specific binding reagents as disclosed in Orlandi et al. (1989; Proc Natl Acad Sci 86:3833-3837) or Winter et al. (1991; Nature 349:293-299). A protein may be used in screening assays of phagemid or B-lymphocyte immunoglobulin libraries to identify antibodies having a desired specificity. Numerous protocols for competitive binding or immunoassays using either polyclonal or monoclonal antibodies with established specificities are well known in the art.

Antibody Specificity

Various methods such as Scatchard analysis combined with radioimmunoassay techniques may be used to assess the affinity of particular antibodies for a protein. Affinity is expressed as an association constant, Kₐ, which is defined as the molar concentration of protein-antibody complex divided by the molar concentrations of free antigen and free antibody under equilibrium conditions. The Kₐ determined for a preparation of polyclonal antibodies, which are heterogeneous in their affinities for multiple antigenic determinants, represents the average affinity, or avidity, of the antibodies. The Kₐ determined for a preparation of monoclonal antibodies, which are specific for a particular antigenic determinant, represents a true measure of affinity. High-affinity antibody preparations with Kₐ ranging from about 10⁹ to 10¹² L/mole are commonly used in immunoassays in which the protein-antibody complex must withstand rigorous manipulations. Low-affinity antibody preparations with Kₐ ranging from about 10⁶ to 10⁷ L/mole are preferred for use in immunopurification and similar procedures which ultimately require dissociation of the protein, preferably in active form, from the antibody (Catty (1988) Antibodies, Volume I: A Practical Approach, IRL Press, Washington D.C.; Liddell and Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing about 5-10 mg specific antibody/ml, is generally employed in procedures requiring precipitation of protein-antibody complexes. Procedures for making antibodies, evaluating antibody specificity, titer, and avidity, and guidelines for antibody quality and usage in various applications, are discussed in Catty (supra) and Ausubel (supra) pp. 11.1-11.31.

Diagnostics

Differential expression of PDE8s, as detected by quantifying PDE8s, a cDNA encoding PDE8s, or an antibody specifically bound to PDE8s, and at least one of the assays below can be used to diagnose and stage neoplastic, immune and neuronal disorders; particularly cancer of the prostate as preceeded by adenofibromatous hyperplasia, lung tumors and type 1 diabetes.

Labeling of Molecules for Assay

A wide variety of reporter molecules and conjugation techniques are known by those skilled in the art and may be used in various nucleic acid, amino acid, and antibody assays. Synthesis of labeled molecules may be achieved using kits such as those supplied by Promega (Madison Wis.) or APB for incorporation of a labeled nucleotide such as ³²P-dCTP (APB), Cy3-dCTP or Cy5-dCTP (Qiagen-Operon, Alameda Calif.), or amino acid such as ³⁵S-methionine (APB). Nucleotides and amino acids may be directly labeled with a variety of substances including fluorescent, chemiluminescent, or chromogenic agents, and the like, by chemical conjugation to amines, thiols and other groups present in the molecules using reagents such as BIODIPY or FITC (Molecular Probes).

Nucleic Acid Assays

The cDNAs, fragments, oligonucleotides, complementary RNAs, and peptide nucleic acids (PNA) may be used to detect and quantify differential gene expression for diagnosis of a disorder. Similarly antibodies which specifically bind PDE8s may be used to quantitate the protein. Disorders associated with such differential expression include cancers of the lung and prostate. The diagnostic assay may use hybridization or amplification technology to compare gene expression in a biological sample from a patient to standard samples in order to detect differential gene expression. Qualitative or quantitative methods for this comparison are well known in the art.

Expression Profiles

An expression profile comprises the expression of a plurality of cDNAs or protein as measured using standard assays with a sample. The cDNAs, proteins or antibodies of the invention may be used as elements on a array to produce an expression profile. In one embodiment, the array is used to diagnose or monitor the progression or treatment of disease.

For example, the cDNA or probe may be labeled by standard methods and added to a biological sample from a patient under conditions for the formation of hybridization complexes. After an incubation period, the sample is washed and the amount of label (or signal) associated with hybridization complexes, is quantified and compared with at least one standard value. If complex formation in the patient sample is altered in comparison to either a normal or disease standard, then differential expression indicates the presence of a disorder.

In order to provide standards for establishing differential expression, normal and disease expression profiles are established. This is accomplished by combining a sample taken from normal subjects, either animal or human, with a cDNA under conditions for hybridization to occur. Standard hybridization complexes may be quantified by comparing the values obtained using normal subjects with values from an experiment in which a known amount of a purified sequence is used. Standard values obtained in this manner may be compared with values obtained from samples from patients who were diagnosed with a particular condition, disease, or disorder. Deviation from standard values toward those associated with a particular disorder is used to diagnose or stage that disorder.

By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disease, or disorder; or treatment of the condition, disease, or disorder. Novel treatment regimens may be tested in these animal models using arrays to establish and then follow expression profiles over time. In addition, arrays may be used with cell cultures or tissues removed from animal models to rapidly screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Such assays may also be used to evaluate the efficacy of a particular therapeutic treatment regimen in animal studies or in clinical trials or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to years.

Protein Assays

Immunological methods for detecting and measuring complex formation as a measure of protein expression using either specific polyclonal or monoclonal antibodies are known in the art. Examples of such techniques include antibody arrays, enzyme-linked immunosorbent assays, fluorescence-activated cell sorting, 2D-PAGE and scintillation counting, protein arrays, radioimmunoassays, and western analysis. Such immunoassays typically involve the measurement of complex formation between the protein and its specific antibody. These assays and their quantitation against purified, labeled standards are well known in the art (Ausubel, supra, unit 10.1-10.6). A two-site, monoclonal-based immunoassay utilizing antibodies reactive to two non-interfering epitopes is preferred, but a competitive binding assay may be employed (Pound (1998) Immunochemical Protocols, Humana Press, Totowa N.J.).

These methods are also useful for diagnosing diseases that show differential protein expression. Normal or standard values for protein expression are established by combining body fluids or cell extracts taken from a normal mammalian or human subject with specific antibodies to a protein under conditions for complex formation. Standard values for complex formation in normal and diseased tissues are established by various methods, often photometric means. Then complex formation as it is expressed in a subject sample is compared with the standard values. Deviation from the normal standard and toward the diseased standard provides parameters for disease diagnosis or prognosis while deviation away from the diseased and toward the normal standard may be used to evaluate treatment efficacy.

Recently, antibody arrays have allowed the development of techniques for high-throughput screening of recombinant antibodies. Such methods use robots to pick and grid bacteria containing antibody genes, and a filter-based ELISA to screen and identify clones that express antibody fragments. Because liquid handling is eliminated and the clones are arrayed from master stocks, the same antibodies can be spotted multiple times and screened against multiple antigens simultaneously. Antibody arrays are highly useful in the identification of differentially expressed proteins. (See de Wildt et al. (2000) Nature Biotechnol 18:989-94.)

Therapeutics

The differential expression of PDE8s is highly associated with diagnosing and staging prostate cancer. In particular, adenofibromatous hyperplasia is an early stage indicator or precursor of prostate cancer. The expression of PDE8s change as prostate cancer progresses. PDE8s are also differentially expressed in lung tumors and type I diabetes. The data for these disease indications have been demonstrated by the transcript imaging and/or microarray analyses presented herein.

When decreased expression or activity of a PDE8 protein is desired, an anti-PDE8 antibody that specifically binds and inhibits PDE8 or an antagonist, inhibitor, a pharmaceutical agent or a composition containing one or more of these molecules may be delivered to a subject in need of such treatment. Such delivery may be effected by methods well known in the art and may include delivery by the antibody claimed herein.

When increased expression or activity of a PDE8 protein is desired, an anti-PDE8 antibody that specifically binds PDE8 may be used to carry an agonist or expression enhancing pharmaceutical agent or a composition containing one or more of these molecules to the tissue of a subject in need of such treatment.

Any of the cDNAs, complementary molecules, RNAi or other fragments thereof, proteins or portions thereof, vectors delivering these nucleic acid molecules or expressing the proteins, therapeutic antibodies, and ligands binding the cDNA or protein may be administered in combination with other therapeutic agents. Selection of the agents for use in combination therapy may be made by one of ordinary skill in the art according to conventional pharmaceutical principles. A combination of therapeutic agents may act synergistically to affect treatment of a particular disorder at a lower dosage of each agent.

Modification of Gene Expression Using Nucleic Acids

Gene expression may be modified by designing complementary or antisense molecules (DNA, RNA, or PNA) to the control, 5′, 3′, or other regulatory regions of the gene encoding PDE8s. Oligonucleotides designed to inhibit transcription initiation are preferred. Similarly, inhibition can be achieved using triple helix base-pairing which inhibits the binding of polymerases, transcription factors, or regulatory molecules (Gee et al. In: Huber and Carr (1994) Molecular and Immunologic Approaches, Futura Publishing, Mt. Kisco N.Y., pp. 163-177). A complementary molecule may also be designed to block translation by preventing binding between ribosomes and mRNA. In one alternative, a library or plurality of cDNAs may be screened to identify those which specifically bind a regulatory, nontranslated sequence.

Ribozymes, enzymatic RNA molecules, may also be used to catalyze the specific cleavage of RNA. The mechanism of ribozyme action involves sequence-specific hybridization of the ribozyme molecule to complementary target RNA followed by endonucleolytic cleavage at sites such as GUA, GUU, and GUC. Once such sites are identified, an oligonucleotide with the same sequence may be evaluated for secondary structural features which would render the oligonucleotide inoperable. The suitability of candidate targets may also be evaluated by testing their hybridization with complementary oligonucleotides using ribonuclease protection assays.

Complementary nucleic acids and ribozymes of the invention may be prepared via recombinant expression, in vitro or in vivo, or using solid phase phosphoramidite chemical synthesis. In addition, RNA molecules may be modified to increase intracellular stability and half-life by addition of flanking sequences at the 5′ and/or 3′ ends of the molecule or by the use of phosphorothioate or 2′ O-methyl rather than phosphodiesterase linkages within the backbone of the molecule. Modification is inherent in the production of PNAs and can be extended to other nucleic acid molecules. Either the inclusion of nontraditional bases such as inosine, queosine, and wybutosine, or the modification of adenine, cytidine, guanine, thymine, and uridine with acetyl-, methyl-, thio-groups renders the molecule more resistant to endogenous endonucleases.

cDNA Therapeutics

The cDNAs of the invention can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a disorder associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

RNA Interference

RNA interference (RNAi), also known as double-stranded RNA (dsRNA)-induced gene silencing, is a method of interfering with the transcription of specific mRNAs through the production of small RNAs (siRNAs) and short hairpin RNAs (shRNAs). These RNAs are naturally formed in a multicomponent nuclease complex (RISC) in the presence of an RNAse III family nuclease (Dicer), and they serve as a guide to identify and destroy complementary transcripts. Transient infection of cells with RNAs capable of interference can bypass the need for Dicer and result in silencing of a gene for the lifespan of the introduced RNAs, usually from about 2 to about 7 days. See Paddison and Hannon (2002) Cancer Cell 2:17-23.

The RNAi pathway is believed to have evolved in early eukaryotes as a cell-based immunity against viral and genetic parasites. It is considered, however, to have great potential as a method of identifying gene function particularly in diseases such as cancer, as well as providing a highly specific means for nucleic acid-based therapies for cancer and other disorders.

Screening and Purification Assays

The cDNA encoding PDE8s may be used to screen a library or a plurality of molecules or compounds for specific binding affinity. The libraries may be antisense molecules, artificial chromosome constructions, branched nucleic acid molecules, DNA molecules, peptides, peptide nucleic acid, proteins such as transcription factors, enhancers, or repressors, RNA molecules, ribozymes, and other ligands which regulate the activity, replication, transcription, or translation of the endogenous gene. The assay involves combining a polynucleotide with a library or plurality of molecules or compounds under conditions allowing specific binding, and detecting specific binding to identify at least one molecule which specifically binds the cDNA.

In one embodiment, the cDNA of the invention may be incubated with a plurality of purified molecules or compounds and binding activity determined by methods well known in the art, e.g., a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay and may be later confirmed by recovering and raising antibodies against that molecule or compound. When these antibodies are added into the assay, they cause a supershift in the gel-retardation assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and collected.

In a further embodiment, the protein or a portion thereof may be used to purify a ligand from a sample. A method for using a protein to purify a ligand would involve combining the protein with a sample under conditions to allow specific binding, detecting specific binding between the protein and ligand, recovering the bound protein, and using a chaotropic agent to separate the protein from the purified ligand.

In a preferred embodiment, PDE8s may be used to screen a plurality of molecules or compounds in any of a variety of screening assays. The portion of the protein employed in such screening may be free in solution, affixed to an abiotic or biotic substrate (e.g. borne on a cell surface), or located intracellularly. For example, in one method, viable or fixed prokaryotic host cells that are stably transformed with recombinant nucleic acids that have expressed and positioned a peptide on their cell surface can be used in screening assays. The cells are screened against a plurality or libraries of ligands, and the specificity of binding or formation of complexes between the expressed protein and the ligand can be measured. Depending on the particular kind of molecules or compounds being screened, the assay may be used to identify agonists, antagonists, antibodies, DNA molecules, small drug molecules, immunoglobulins, inhibitors, mimetics, peptides, peptide nucleic acids, proteins, and RNA molecules or any other ligand, which specifically binds the protein.

In one aspect, this invention contemplates a method for high throughput screening using very small assay volumes and very small amounts of test compound as described in U.S. Pat. No. 5,876,946, incorporated herein by reference. This method is used to screen large numbers of molecules and compounds via specific binding. In another aspect, this invention also contemplates the use of competitive drug screening assays in which neutralizing antibodies capable of binding the protein specifically compete with a test compound capable of binding to the protein. Molecules or compounds identified by screening may be used in a mammalian model system to evaluate their toxicity or therapeutic potential.

Pharmaceutical Compositions

Pharmaceutical compositions may be formulated and administered, to a subject in need of such treatment, to attain a therapeutic effect. Such compositions contain the instant protein, agonists, antagonists, small drug molecules, immunoglobulins, inhibitors, mimetics, multispecific molecules, peptides, peptide nucleic acids, pharmaceutical agent, proteins, and RNA molecules. Compositions may be manufactured by conventional means such as mixing, dissolving, granulating, dragee-making, levigating, emulsifying, encapsulating, entrapping, or lyophilizing. The composition may be provided as a salt, formed with acids such as hydrochloric, sulfuric, acetic, lactic, tartaric, malic, and succinic, or as a lyophilized powder which may be combined with a sterile buffer such as saline, dextrose, or water. These compositions may include auxiliaries or excipients which facilitate processing of the active compounds.

Auxiliaries and excipients may include coatings, fillers or binders including sugars such as lactose, sucrose, mannitol, glycerol, or sorbitol; starches from corn, wheat, rice, or potato; proteins such as albumin, gelatin and collagen; cellulose in the form of hydroxypropylmethyl-cellulose, methyl cellulose, or sodium carboxymethylcellulose; gums including arabic and tragacanth; lubricants such as magnesium stearate or talc; disintegrating or solubilizing agents such as the, agar, alginic acid, sodium alginate or cross-linked polyvinyl pyrrolidone; stabilizers such as carbopol gel, polyethylene glycol, or titanium dioxide; and dyestuffs or pigments added for identify the product or to characterize the quantity of active compound or dosage.

These compositions may be administered by any number of routes including oral, intravenous, intramuscular, intra-arterial, intramedullary, intrathecal, intraventricular, transdermal, subcutaneous, intraperitoneal, intranasal, enteral, topical, sublingual, or rectal.

The route of administration and dosage will determine formulation; for example, oral administration may be accomplished using tablets, pills, dragees, capsules, liquids, gels, syrups, slurries, or suspensions; parenteral administration may be formulated in aqueous, physiologically compatible buffers such as Hanks' solution, Ringer's solution, or physiologically buffered saline. Suspensions for injection may be aqueous, containing viscous additives such as sodium carboxymethyl cellulose or dextran to increase the viscosity, or oily, containing lipophilic solvents such as sesame oil or synthetic fatty acid esters such as ethyl oleate or triglycerides, or liposomes. Penetrants well known in the art are used for topical or nasal administration.

Toxicity and Therapeutic Efficacy

A therapeutically effective dose refers to the amount of active ingredient which ameliorates symptoms or condition. For any compound, a therapeutically effective dose can be estimated from cell culture assays using normal and neoplastic cells or in animal models. Therapeutic efficacy, toxicity, concentration range, and route of administration may be determined by standard pharmaceutical procedures using experimental animals.

The therapeutic index is the dose ratio between therapeutic and toxic effects—LD50 (the dose lethal to 50% of the population)/ED50 (the dose therapeutically effective in 50% of the population)—and large therapeutic indices are preferred. Dosage is within a range of circulating concentrations, includes an ED50 with little or no toxicity, and varies depending upon the composition, method of delivery, sensitivity of the patient, and route of administration. Exact dosage will be determined by the practitioner in light of factors related to the subject in need of the treatment.

Dosage and administration are adjusted to provide active moiety that maintains therapeutic effect. Factors for adjustment include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular composition.

Normal dosage amounts may vary from 0.1 μg, up to a total dose of about 1 g, depending upon the route of administration. The dosage of a particular composition may be lower when administered to a patient in combination with other agents, drugs, or hormones. Guidance as to particular dosages and methods of delivery is provided in the pharmaceutical literature. Further details on techniques for formulation and administration may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, gestation period, numbers of progeny, and abundant reference literature. Inbred and outbred rodent strains provide a convenient model for investigation of the physiological consequences of under- or over-expression of genes of interest and for the development of methods for diagnosis and treatment of diseases. A mammal inbred to over-express a particular gene (for example, secreted in milk) may also serve as a convenient source of the protein expressed by that gene.

Toxicology

Toxicology is the study of the effects of agents on living systems. The majority of toxicity studies are performed on rats or mice. Observation of qualitative and quantitative changes in physiology, behavior, homeostatic processes, and lethality in the rats or mice are used to generate a toxicity profile and to assess consequences on human health following exposure to the agent.

Genetic toxicology identifies and analyzes the effect of an agent on the rate of endogenous, spontaneous, and induced genetic mutations. Genotoxic agents usually have common chemical or physical properties that facilitate interaction with nucleic acids and are most harmful when chromosomal aberrations are transmitted to progeny. Toxicological studies may identify agents that increase the frequency of structural or functional abnormalities in the tissues of the progeny if administered to either parent before conception, to the mother during pregnancy, or to the developing organism. Mice and rats are most frequently used in these tests because their short reproductive cycle allows the production of the numbers of organisms needed to satisfy statistical requirements.

Acute toxicity tests are based on a single administration of an agent to the subject to determine the symptomology or lethality of the agent. Three experiments are conducted: 1) an initial dose-range-finding experiment, 2) an experiment to narrow the range of effective doses, and 3) a final experiment for establishing the dose-response curve.

Subchronic toxicity tests are based on the repeated administration of an agent. Rat and dog are commonly used in these studies to provide data from species in different families. With the exception of carcinogenesis, there is considerable evidence that daily administration of an agent at high-dose concentrations for periods of three to four months will reveal most forms of toxicity in adult animals.

Chronic toxicity tests, with a duration of a year or more, are used to test whether long term administration may elicit toxicity, teratogenesis, or carcinogenesis. When studies are conducted on rats, a minimum of three test groups plus one control group are used, and animals are examined and monitored at the outset and at intervals throughout the experiment.

Transgenic Animal Models

Transgenic rodents that over-express or under-express a gene of interest may be inbred and used to model human diseases or to test therapeutic or toxic agents. (See, e.g., U.S. Pat. No. 5,175,383 and U.S. Pat. No. 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the ability to form embryonic tissues. When ES cells are placed inside a carrier embryo, they resume normal development and contribute to tissues of the live-born animal. ES cells are the preferred cells used in the creation of experimental knockout and knockin rodent strains. Mouse ES cells, such as the mouse 129/SvJ cell line, are derived from the early mouse embryo and are grown under culture conditions well known in the art. Vectors used to produce a transgenic strain contain a disease gene candidate and a marker gene, the latter serves to identify the presence of the introduced disease gene. The vector is transformed into ES cells by methods well known in the art, and transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains.

ES cells derived from human blastocysts may be manipulated in vitro to differentiate into at least eight separate cell lineages. These lineages are used to study the differentiation of various cell types and tissues in vitro, and they include endoderm, mesoderm, and ectodermal cell types which differentiate into, for example, neural cells, hematopoietic lineages, and cardiomyocytes.

Knockout Analysis

In gene knockout analysis, a region of a gene is enzymatically modified to include a non-mammalian gene such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene. Transformed cells are injected into rodent blastulae, and the blastulae are implanted into pseudopregnant dams. Transgenic progeny are crossbred to obtain homozygous inbred lines which lack a functional copy of the mammalian gene. In one example, the mammalian gene is a human gene.

Knockin Analysis

ES cells can be used to create knockin humanized animals (pigs) or transgenic animal models (mice or rats) of human diseases. With knockin technology, a region of a human gene is injected into animal ES cells, and the human sequence integrates into the animal cell genome. Transformed cells are injected into blastulae and the blastulae are implanted as described above. Transgenic progeny or inbred lines are studied and treated with pharmaceutical agents to obtain information on treatment of the analogous human condition. These methods have been used to model several human diseases.

Non-Human Primate Model

The field of animal testing deals with data and methodology from basic sciences such as physiology, genetics, chemistry, pharmacology and statistics. These data are paramount in evaluating the effects of therapeutic agents on non-human primates as they can be related to human health. Monkeys are used as human surrogates in vaccine and drug evaluations, and their responses are relevant to human exposures under similar conditions. Cynomolgus and Rhesus monkeys (Macaca fascicularis and Macaca mulatta, respectively) and Common Marmosets (Callithrix jacchus) are the most common non-human primates (NHPs) used in these investigations. Since great cost is associated with developing and maintaining a colony of NHPs, early research and toxicological studies are usually carried out in rodent models. In studies using behavioral measures such as drug addiction, NHPs are the first choice test animal. In addition, NHPs and individual humans exhibit differential sensitivities to many drugs and toxins and can be classified as a range of phenotypes from “extensive metabolizers” to “poor metabolizers” of these agents.

In additional embodiments, the cDNAs which encode the protein may be used in any molecular biology techniques that have yet to be developed, provided the new techniques rely on properties of cDNAs that are currently known, including, but not limited to, such properties as the triplet genetic code and specific base pair interactions.

### EXAMPLES

I cDNA Library Construction

THP1PLB02

The THP1PLB02 cDNA library was constructed by reamplification of THP1PLB01. The THP1PLB01 cDNA library was made from activated human monocytes by Stratagene (La Jolla Calif.). Poly(A+)RNA was purified from THP-1 cells which were cultured for 48 hr with 100 nm TPA and activated with 1 μg/ml LPS after 4 hr. cDNA synthesis was primed separately with both oligo d(T) and random hexamers. Synthetic adaptor oligonucleotides were ligated onto cDNA ends enabling insertion into the UNI-ZAP vector system (Stratagene). After construction, the two libraries were combined into a single library by mixing equal numbers of bacteriophages.

The cDNA library was screened with either DNA probes or antibody probes and the pBLUESCRIPT phagemid (Stratagene) was excised in vivo. The custom-constructed library phage particles were transfected into E. coli host strain XL1-BLUE (Stratagene). Alternative unidirectional vectors include, but are not limited to, pcDNAI (Invitrogen, San Diego Calif.) and pSHlox-1 (Novagen, Madison Wis.).

LATRNOT01

The LATRNOT01 cDNA library was obtained from left ventricle tissue from a 51 year-old Caucasian female (Lot No. RU95-03-196, International Institute for Advanced Medicine, Jessup Pa.). The tissue was flash frozen and ground with mortar and pestle. The tissue was lysed immediately in buffer containing guanidinium isothiocyanate and spun through cesium chloride. The precipitate was treated by several phenol chloroform extractions and ethanol precipitated at pH 8. The polyadenylated mRNA was isolated, treated with DNAse, and purified using OLIGOTEX kit (Qiagen, Chatsworth Calif.)

First strand cDNA synthesis was accomplished using an oligo d(T) primer/linker which also contained an XhoI restriction site. Second strand synthesis was performed using a combination of DNA polymerase I, E. coli ligase, and RNAse H, followed by the addition of an EcoRI adaptor to the blunt-ended cDNA. The EcoRI adapted, double-stranded cDNA was then digested with XhoI restriction enzyme and fractionated to obtain sequences which exceeded 800 bp in size. The cDNAs were inserted into the LAMBDAZAP vector system (Stratagene); and the vector which contained the pBLUESCRIPT phagemid (Stratagene) was transformed into E. coli host cells strain XL1-BLUEMRF (Stratagene).

II Isolation, Preparation, and Sequencing of cDNAs

THP1PLB02 and LATRNOT01

The phagemid forms of individual cDNA clones were obtained by the in vivo excision process, in which the host bacterial strain was coinfected with both the lambda library phage and an f1 helper phage. Enzymes derived from both the library-containing phage and the helper phage nicked the λ DNA, initiated new DNA synthesis from defined sequences on the λ target DNA, and created a smaller, single stranded circular phagemid DNA molecule that included all DNA sequences of the pBLUESCRIPT plasmid and the cDNA insert. The phagemid DNA was secreted from the cells, purified, and used to re-infect fresh host cells, where double stranded phagemid DNA was produced. Because the phagemid carries the gene for β-lactamase, the newly-transformed bacteria were selected on a medium containing ampicillin.

The THP1PLB02 phagemid DNA was purified using the MAGIC MINIPREPS DNA purification system (Promega). The LATRNOT01 plasmid DNA was released from the cells and purified using the MINIPREP kit (Advanced Genetic Technologies Corporation, Gaithersburg Md.). This kit consists of a 96 well block with reagents for 960 purifications. Alternatively, phagemid DNA was purified using the QIAwell-8 Plasmid, QIAwell PLUS, and QIAwell ULTRA DNA purification system (Qiagen).

The cDNAs were sequenced by the method of Sanger and Coulson (1975; J Mol Biol 94:441-8), using a MICROLAB 2200 (Hamilton, Reno Nev.) in combination with DNA ENGINE thermal cyclers (MJ Research, Watertown Mass.) and PRISM 377 or 373 DNA sequencing systems (ABI).

III Extension of cDNAs

cDNA sequences containing 5′ extensions of the ESTs were extended by PCR amplification using human λgt10 testis or stomach cDNA libraries (Clontech) and nested primers. For each reaction, 2.5×10⁷ pfu were boiled for 5 minutes to release DNA. First round PCR (15 cycles) was performed with a PDE8A specific primer (8A specific-outer: 5′-GAAGCACATCAGCAGAAT-3′, SEQ ID NO:18) and either a λgt10 forward (5′-TCGCTTAGTTTTACCGTTTTC-3′, SEQ ID NO:19, or a λgt10 reverse (5′-TATCGCCTCCATCAACAAACTT-3, SEQ ID NO:20) primer. An aliquot, 1/50 of the reaction mixture, was used as a template for a second round of amplification (30 cycles) with a PDE8A specific primer (8A specific-inner: 5′-TTGTGGTAGGGATTGGAG-3′, SEQ ID NO:21) with either a nested λgt10 forward (5′-AGCAAGTTCAGCCTGGTTAAG-3′, SEQ ID NO:22) or λgt10 reverse (5′-CTTATGAGTATTTCTTCCAGGGTA-3′, SEQ ID NO:23) primer. Southern analysis of the PCR products used an internal PDE8A hybridization probe (5′-ATCATGGTTACAAATTATCGAAGCCAATTA-3′, SEQ ID NO:24). Positive bands were subcloned and sequenced. All sequences subsequently incorporated into the sequence encoding PDE8A(E) were verified by sequencing multiple independent PCR amplifications from the cDNA library DNA using unique primers, or by independent amplification from mRNA.

IV Homology Searching of cDNA Clones and Their Deduced Proteins

The cDNAs of the Sequence Listing or their deduced amino acid sequences were used to query databases such as GenBank, SwissProt, BLOCKS, and the like. These databases that contain previously identified and annotated sequences or domains were searched using BLAST or BLAST2 to produce alignments and to determine which sequences were exact matches or homologs. The alignments were to sequences of prokaryotic (bacterial) or eukaryotic (animal, fungal, or plant) origin. Alternatively, algorithms such as the one described in Smith and Smith (1992, Protein Engineering 5:35-51) could have been used to deal with primary sequence patterns and secondary structure gap penalties. All of the sequences disclosed in this application have lengths of at least 49 nucleotides, and no more than 12% uncalled bases (where N is recorded rather than A, C, G, or T).

As detailed in Karlin and Altschul (1993; Proc Natl Acad Sci 90:5873-5877), BLAST matches between a query sequence and a database sequence were evaluated statistically and only reported when they satisfied the threshold of 10⁻²⁵ for nucleotides and 10⁻¹⁴ for peptides. Homology was also evaluated by product score calculated as follows: the % nucleotide or amino acid identity [between the query and reference sequences] in BLAST is multiplied by the % maximum possible BLAST score [based on the lengths of query and reference sequences] and then divided by 100. In comparison with hybridization procedures used in the laboratory, the stringency for an exact match was set from a lower limit of about 40 (with 1-2% error due to uncalled bases) to a 100% match of about 70.

The BLAST software suite (NCBI, Bethesda Md.), includes various sequence analysis programs including “blastn” that is used to align nucleotide sequences and BLAST2 that is used for direct pairwise comparison of either nucleotide or amino acid sequences. BLAST programs are commonly used with gap and other parameters set to default settings, e.g.: Matrix: BLOSUM62; Reward for match: 1; Penalty for mismatch: −2; Open Gap: 5 and Extension Gap: 2 penalties; Gap×drop-off: 50; Expect: 10; Word Size: 11; and Filter: on. Identity is measured over the entire length of a sequence. Brenner (supra) analyzed BLAST for its ability to identify structural homologs by sequence identity and found 30% identity is a reliable threshold for sequence alignments of at least 150 residues and 40%, for alignments of at least 70 residues.

The cDNAs of this application were compared with assembled consensus sequences or templates found in the LIFESEQ GOLD database (Incyte Genomics). Component sequences from cDNA, extension, full length, and shotgun sequencing projects were subjected to PHRED analysis and assigned a quality score. All sequences with an acceptable quality score were subjected to various pre-processing and editing pathways to remove low quality 3′ ends, vector and linker sequences, polyA tails, Alu repeats, mitochondrial and ribosomal sequences, and bacterial contamination sequences. Edited sequences had to be at least 50 bp in length, and low-information sequences and repetitive elements such as dinucleotide repeats, Alu repeats, and the like, were replaced by “Ns” or masked.

Edited sequences were subjected to assembly procedures in which the sequences were assigned to gene bins. Each sequence could only belong to one bin, and sequences in each bin were assembled to produce a template. Newly sequenced components were added to existing bins using BLAST and CROSSMATCH. To be added to a bin, the component sequences had to have a BLAST quality score greater than or equal to 150 and an alignment of at least 82% local identity. The sequences in each bin were assembled using PHRAP. Bins with several overlapping component sequences were assembled using DEEP PHRAP. The orientation of each template was determined based on the number and orientation of its component sequences.

Bins were compared to one another, and those having local similarity of at least 82% were combined and reassembled. Bins having templates with less than 95% local identity were split. Templates were subjected to analysis by STITCHER/EXON MAPPER algorithms that determine the probabilities of the presence of splice variants, alternatively spliced exons, splice junctions, differential expression of alternative spliced genes across tissue types or disease states, and the like. Assembly procedures were repeated periodically, and templates were annotated using BLAST against GenBank databases such as GBpri. An exact match was defined as having from 95% local identity over 200 base pairs through 100% local identity over 100 base pairs and a homology match as having an E-value (or probability score) of ≦1×10⁻⁸. The templates were also subjected to frameshift FASTx against GENPEPT, and homology match was defined as having an E-value of ≦1×10⁻⁸. Template analysis and assembly was described in U.S. Ser. No. 09/276,534, filed Mar. 25, 1999.

Following assembly, templates were subjected to BLAST, motif, and other functional analyses and categorized in protein hierarchies using methods described in U.S. Ser. No. 08/812,290 and U.S. Ser. No. 08/811,758, both filed Mar. 6, 1997; in U.S. Ser. No. 08/947,845, filed Oct. 9, 1997; and in U.S. Ser. No. 09/034,807, filed Mar. 4, 1998. Then templates were analyzed by translating each template in all three forward reading frames and searching each translation against the PFAM database of hidden Markov model-based protein families and domains using the HMMER software package (Washington University School of Medicine, St. Louis Mo.). The cDNA was further analyzed using MACDNASIS PRO software (Hitachi Software Engineering), and LASERGENE software (DNASTAR) and queried against public databases such as the GenBank rodent, mammalian, vertebrate, prokaryote, and eukaryote databases, SwissProt, BLOCKS, PRINTS, PFAM, and Prosite.

V Northern Analysis, Transcript Imaging, and Guilt-By-Association

Northern Analysis

Northern analysis is a laboratory technique used to detect the presence of a transcript of a gene and involves the hybridization of a labeled nucleotide sequence to a membrane on which RNAs from a particular cell type or tissue have been bound. The technique is described in EXAMPLE VII below and in Ausubel, supra, units 4.1-4.9)

Human multiple tissue northern blots (Clontech, Palo Alto Calif.) were hybridized with a probe consisting of the 5′ most 939 nucleotides of Incyte clone 156196. Probe DNA was labeled with ³²P using the “Ready-To-Go” random prime labeling kit (APB) and washed to a stringency of 0.5×SSC, 65° C. The highest levels of PDE8A were expressed in testis, ovary, small intestine, and colon, but detectable levels were seen in heart, brain, placenta, lung, liver, skeletal muscle, kidney, pancreas, spleen, and prostate (data not shown).

Analogous computer techniques applying BLAST are used to search for identical or related molecules in nucleotide databases such as GenBank or the LIFESEQ database (Incyte Genomics). This analysis is faster than multiple membrane-based hybridizations. In addition, the sensitivity of the computer search can be modified to determine whether any particular match is categorized as exact or homologous. The basis of the search is the product score which was described above in EXAMPLE IV.

The results of northern analysis are reported as a list of libraries in which the transcript encoding PDE8s occurs. Abundance and percent abundance are also reported. Abundance directly reflects the number of times a particular transcript is represented in a cDNA library, and percent abundance is abundance divided by the total number of sequences examined in the cDNA library.

Transcript Imaging

A transcript image was performed using the LIFESEQ GOLD database (Incyte Genomics). This allowed assessment of the relative abundance of expressed polynucleotides in all of the cDNA libraries and was described in U.S. Pat. No. 5,840,484, incorporated herein by reference. All sequences and cDNA libraries in the LIFESEQ database are categorized by system, organ/tissue and cell type. The categories include cardiovascular system, connective tissue, digestive system, embryonic structures, endocrine system, exocrine glands, female and male genitalia, germ cells, hemic/immune system, liver, musculoskeletal system, nervous system, pancreas, respiratory system, sense organs, skin, stomatognathic system, unclassified/mixed, and the urinary tract. Criteria for transcript imaging can be selected from category, number of cDNAs per library, library description, disease indication, clinical relevance of sample, and the like.

For each library, the number of cDNAs were counted and shown over the total number of cDNAs in that library. In some transcript images, all enriched, normalized or subtracted libraries, which have high copy number sequences removed prior to processing, and all mixed or pooled tissues, which are considered non-specific in that they contain more than one tissue type or more than one subject's tissue, can be excluded from the analysis. Treated and untreated cell lines and/or fetal tissue data can also be excluded where clinical relevance is emphasized. Conversely, fetal tissue can be emphasized wherever elucidation of inherited disorders or differentiation of particular adult or embryonic stem cells into tissues or organs (such as heart, kidney, nerves or pancreas) would be aided by removing clinical samples from the analysis. Transcript imaging can also be used to support data from other methodologies such as hybridization, guilt-by-association and array technologies.

The transcript images for prostate and pancreas are shown in the tables below. The first column of each table shows the library name; the second column, the number of cDNAs in that library; the third column, the description of the tissue from which the library was made; the fourth column, the absolute abundance (Abund) of transcript expression in that library; and the fifth column, the percentage abundance (% Abund) of transcript expression. The protein or transcript was at differentially expressed adenofibromatous hyperplasia and in early stage prostate cancers.

SEQ ID NO:1 and 8

Category: Prostate

| Library* | cDNAs | Library Description** | Abund | % Abund |
|---|---|---|---|---|
| PROSTME06 | 3718 | prostate, AH, mw/adenoCA, 57M, 5RP | 1 | 0.0269 |
| PROSBPT02 | 6583 | prostate, AH, mw/adenoCA, 65M | 1 | 0.0152 |
| PROSTMT02 | 3885 | prostate, AH, mw/adenoCA, 66M, m/PROSTUT17 | 4 | 0.103 |
| PROSTUT08 | 3751 | prostate tumor, adenoCA, 60M, m/PROSNOT14 | 3 | 0.08 |
| PROSNOT05 | 1793 | prostate, AH, mw/adenoCA, 67M, m/PROSTUT03 | 1 | 0.0558 |
| PROSTMC01 | 3881 | prostate, AH, mw/adenoCA, 55M, m/PROSTUT16 | 2 | 0.0515 |
| PROSNOT15 | 4131 | prostate, AH, mw/adenoCA, 66M, m/PROSTUT10 | 2 | 0.0484 |
| PROSTMT07 | 3103 | prostate, AH, mw/adenoCA, 73M | 1 | 0.0322 |
| PROSTMY01 | 6458 | prostate, AH, mw/adenoCA, node mets, 55M, m/PROSTUT16 | 2 | 0.031 |
| PROSTUT10 | 6969 | prostate tumor, adenoCA, 66M, m/PROSNOT15, PROSDIN01 | 2 | 0.0287 |
| PROETUP02 | 3607 | prostate tumor, cancer, 45M, m/PROETMP01/02, CGAP | 1 | 0.0277 |
| PROSBPT07 | 3625 | prostate, AH, mw/adenoCA, 53M | 1 | 0.0276 |
| PROSTME06 | 3718 | prostate, AH, mw/adenoCA, 57M, 5RP | 1 | 0.0269 |
| PROSTUT20 | 3744 | prostate tumor, adenoCA, 58M | 1 | 0.0267 |
| PROSTMT03 | 3815 | prostate, mw/adenoCA, 68M, m/PROSTUT18 | 1 | 0.0262 |
| PROETMP02 | 3879 | prostate, epithelium, PIN, mw/cancer, 45M, m/PROETUP02 | 1 | 0.0258 |
| PROSNOT06 | 8800 | prostate, AH, mw/adenoCA, 57M, m/PROSTUT04 | 2 | 0.0227 |
| PROSTUT12 | 7128 | prostate tumor, adenoCA, 65M, m/PROSNOT20 | 1 | 0.014 |
| PROSTUT04 | 8513 | prostate tumor, adenoCA, 57M, m/PROSNOT06 | 1 | 0.0117 |

*Normalized, pooled, and subtracted libraries and those with less than 1000 cDNAs were removed from the analysis.
**The first two samples reflect data attributable only to SEQ ID NO: 8
Abbreviations are
AH = adenofibromatous hyperplasia,
CA = carcinoma or cancer, and
m/ or mw/ = matched with.

PDE8 expression was not found in benign prostatic hyperplasia (PROSBPT03) or normal prostates from young 21-32 year old males who died accidentally (PROSNOP01, PROSNOP03, PROSNOP07, and PROSNOT11); therefore SEQ ID NO:1 and SEQ ID NO:8 are diagnostic of AH as an early indicator of prostate cancer.

Category: Pancreas

| Library* | cDNAs | Description of Tissue | Abundance | % Abundance |
|---|---|---|---|---|
| PANCNOT23 | 3916 | pancreas, type I diabetes, 43F | 1 | 0.0255 |
| PANCTUP03 | 22650 | pancreas tumor, adenoCA, 3′ CGAP | 2 | 0.0088 |

*All tissues were included in the analysis.

SEQ ID NO:1 was expressed in two pancreatic tissues; in type 1 diabetes, percent abundance was 3-fold higher than in adenocarcinoma of the pancreas. SEQ ID NO:1 was not expressed in type II diabetes (PANCDIT03), normal pancreas (PANCNOE02, PANCNON03, PANCNOP03, PANCNOP05, PANCNOT01, PANCNOT04, PANCNOT05, PANCNOT07, PANCNOT16, PANCNOT17, PANCNOT19, PANCNOT21, PANCNOT22), pancreatitis (PANCNOT08), islet cell hyperplasia (PANCNOT15), pancreatic tumor line (PANCTUM01), or pancreatic tumors (PANCTUP0, PANCTUP02, PANCTUT01, and PANCTUT02); therefore SEQ ID NO:1 is diagnostic of type 1 diabetes.

Guilt-By-Association

GBA identifies cDNAs that are expressed in a plurality of cDNA libraries relating to a specific disease process, subcellular compartment, cell type, tissue type, or species. The expression patterns of cDNAs with unknown function are compared with the expression patterns of genes having well documented function to determine whether a specified co-expression probability threshold is met. Through this comparison, a subset of the cDNAs having a highly significant co-expression probability with the known genes are identified.

The cDNAs originate from human cDNA libraries from any cell or cell line, tissue, or organ and may be selected from a variety of sequence types including, but not limited to, expressed sequence tags (ESTs), assembled polynucleotides, full length gene coding regions, promoters, introns, enhancers, 5′ untranslated regions, and 3′ untranslated regions. To have statistically significant analytical results, the cDNAs need to be expressed in at least five cDNA libraries. The number of cDNA libraries whose sequences are analyzed can range from as few as 500 to greater than 10,000.

The method for identifying cDNAs that exhibit a statistically significant co-expression pattern is as follows. First, the presence or absence of a gene in a cDNA library is defined: a gene is present in a library when at least one fragment of its sequence is detected in a sample taken from the library, and a gene is absent from a library when no corresponding fragment is detected in the sample.

Second, the significance of co-expression is evaluated using a probability method to measure a due-to-chance probability of the co-expression. The probability method can be the Fisher exact test, the chi-squared test, or the kappa test. These tests and examples of their applications are well known in the art and can be found in standard statistics texts (Agresti (1990) Categorical Data Analysis, John Wiley & Sons, New York N.Y.; Rice (1988) Mathematical Statistics and Data Analysis, Duxbury Press, Pacific Grove Calif.). A Bonferroni correction (Rice, supra, p. 384) can also be applied in combination with one of the probability methods for correcting statistical results of one gene versus multiple other genes. In a preferred embodiment, the due-to-chance probability is measured by a Fisher exact test, and the threshold of the due-to-chance probability is set preferably to less than 0.001.

This method of estimating the probability for co-expression of two genes assumes that the libraries are independent and are identically sampled. However, in practical situations, the selected cDNA libraries are not entirely independent because: 1) more than one library may be obtained from a single subject or tissue, and 2) different numbers of cDNAs, typically ranging from 5,000 to 10,000, may be sequenced from each library. In addition, since a Fisher exact co-expression probability is calculated for each gene versus every other gene that occurs in at least five libraries, a Bonferroni correction for multiple statistical tests is used (See Walker et al. (1999; Genome Res 9:1198-203; expressly incorporated herein by reference).

VI Chromosome Mapping

Radiation hybrid and genetic mapping data available from public resources such as the Stanford Human Genome Center (SHGC), Whitehead Institute for Genome Research (WIGR), and Généthon are used to determine if any of the cDNAs presented in the Sequence Listing have been mapped. Any of the fragments of the cDNA encoding PDE8s that have been mapped result in the assignment of all related regulatory and coding sequences to the same location. The genetic map locations are described as ranges, or intervals, of human chromosomes. The map position of an interval, in cM (which is roughly equivalent to 1 megabase of human DNA), is measured relative to the terminus of the chromosomal p-arm.

VII Hybridization and Amplication Technologies and Analyses

Sample Preparation

The normal and cancerous tissue samples presented in the lung microarray data are described by donor identification number in the table below. The first column shows the donor ID; the second, a description of the cancerous tissue; the third column, stage of the cancer, the fourth column, donor age/sex; and the fifth column, the % overt tumor cells in the biopsy. All of the lung tissues below were obtained from the Roy Castle International Centre for Lung Cancer Research (Liverpool UK).

| | | | | % tumor |
|---|---|---|---|---|
| Donor ID | Description of the Cancerous Tissue | Stage | Age/Sex | cells |
| 7175 | Moderately differentiated, adenocarcinoma | IB | 67M | 50 |
| 7965 | Moderately differentiated adenocarcinoma | IIIA | 54F | 60 |
| 7966 | Moderately differentiated adenocarcinoma | IIIA | 39F | 10 |
| 7972 | Moderately differentiated squamous cell carcinoma | IIIA | 62M | 10 |
| 7973 | Poorly differentiated adenocarcinoma | IIIA | 54M | 60 |

The prostatic cell lines, their descriptions and treatments are presented in the table below.

| Prostate Sample | Description |
|---|---|
| PrEC Cells | Non-malignant |
| PZHPV7 Line | Non-malignant |
| CAHPV10 Line | Prostate CA, |
| | non-tumorigenic |
| DU145 Line | Prostate CA, metastatic |
| DU145 Line, PMA + Ionomycin (timecourse) | Prostate CA, metastatic |
| PC3 Line | Prostate CA, metastatic |
| PC3 Line, PMA + Ionomycin (timecourse) | Prostate CA, metastatic |

Cell lines were obtained from ATCC (Manassus, VA).

Immobilization of cDNAs on a Substrate

The cDNAs are applied to a substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37 C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2×SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above. Purified nucleic acids are robotically arranged and immobilized on polymer-coated glass slides using the procedure described in U.S. Pat. No. 5,807,522. Polymer-coated slides are prepared by cleaning glass microscope slides (Corning Life Sciences) by ultrasound in 0.1% SDS and acetone, etching in 4% hydrofluoric acid (VWR Scientific Products, West Chester Pa.), coating with 0.05% aminopropyl silane (Sigma-Aldrich) in 95% ethanol, and curing in a 110 C. oven. The slides are washed extensively with distilled water between and after treatments. The nucleic acids are arranged on the slide and then immobilized by exposing the array to UV irradiation using a STRATALINKER UV-crosslinker (Stratagene). Arrays are then washed at room temperature in 0.2% SDS and rinsed three times in distilled water. Non-specific binding sites are blocked by incubation of arrays in 0.2% casein in phosphate buffered saline (PBS; Tropix, Bedford Mass.) for 30 min at 60 C.; then the arrays are washed in 0.2% SDS and rinsed in distilled water as before.

Probe Preparation for Membrane Hybridization

Hybridization probes derived from the cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100 C. for five min, and briefly centrifuging. The denatured cDNA is then added to a REDIPRIME tube (APB), gently mixed until blue color is evenly distributed, and briefly centrifuged. Five μl of [³²P]dCTP is added to the tube, and the contents are incubated at 37 C. for 10 min. The labeling reaction is stopped by adding 5 μl of 0.2M EDTA, and probe is purified from unincorporated nucleotides using a PROBEQUANT G-50 microcolumn (APB). The purified probe is heated to 100 C. for five min, snap cooled for two min on ice, and used in membrane-based hybridizations as described below.

Probe Preparation for QPCR

Probes for the QPCR were prepared according to the ABI protocol.

Probe Preparation for Polymer Coated Slide Hybridization

The following method was used for the preparation of probes for the microarray analysis presented in FIG. 3. Hybridization probes derived from mRNA isolated from samples are employed for screening cDNAs of the Sequence Listing in array-based hybridizations. Probe is prepared using the GEMbright kit (Incyte Genomics) by diluting mRNA to a concentration of 200 ng in 9 μl TE buffer and adding 5 μl 5×buffer, 1 μl 0.1 M DTT, 3 μl Cy3 or Cy5 labeling mix, 1 μl RNAse inhibitor, 1 μl reverse transcriptase, and 5 μl 1×yeast control mRNAs. Yeast control mRNAs are synthesized by in vitro transcription from noncoding yeast genomic DNA (W. Lei, unpublished). As quantitative controls, one set of control mRNAs at 0.002 ng, 0.02 ng, 0.2 ng, and 2 ng are diluted into reverse transcription reaction mixture at ratios of 1:100,000, 1:10,000, 1:1000, and 1:100 (w/w) to sample mRNA respectively. To examine mRNA differential expression patterns, a second set of control mRNAs are diluted into reverse transcription reaction mixture at ratios of 1:3, 3:1, 1:10, 10:1, 1:25, and 25:1 (w/w). The reaction mixture is mixed and incubated at 37 C. for two hr. The reaction mixture is then incubated for 20 min at 85 C., and probes are purified using two successive CHROMA SPIN+TE 30 columns (Clontech, Palo Alto Calif.). Purified probe is ethanol precipitated by diluting probe to 90 μl in DEPC-treated water, adding 2 μl 1 mg/ml glycogen, 60 μl 5 M sodium acetate, and 300 μl 100% ethanol. The probe is centrifuged for 20 min at 20,800×g, and the pellet is resuspended in 12 μl resuspension buffer, heated to 65 C. for five min, and mixed thoroughly. The probe is heated and mixed as before and then stored on ice. Probe is used in high density array-based hybridizations as described below.

In situ Hybridization

In situ hybridization is used to determine the expression of PDE8s in sectioned tissue. With the digoxygenin protocol, fresh cryosections, 10 microns thick, are removed from the freezer, immediately immersed in 4% paraformaldehyde for 10 min, rinsed in PBS, and acetylated in 0.1 M TEA, pH 8.0, containing 0.25% (v/v) acetic anhydride. After the tissue equilibrated in 5×SSC, it is prehybridized in hybridization buffer (50% formamide, 5×SSC, 1×Denhardt's solution, 10% dextran sulfate, and 1 mg/ml herring sperm DNA).

Digoxygenin-labeled PDE8s-specific RNA probes, sense and antisense nucleotides selected from the cDNA of SEQ ID NO:1 are produced using PCR. Approximately 500 ng/ml of probe is used in overnight hybridizations at 65 C. in hybridization buffer. Following hybridization, the sections are rinsed for 30 min in 2'SSC at room temperature, 1 hr in 2×SSC at 65 C., and 1 hr in 0.1'SSC at 65 C. The sections are equilibrated in PBS, blocked for 30 min in 10% DIG kit blocker (Roche Molecular Biochemicals, Indianapolis Ind.) in PBS, then incubated overnight at 4 C. in 1:500 anti-DIG-AP. The following day, the sections are rinsed in PBS, equilibrated in detection buffer (0.1 M Tris, 0.1 M NaCl, 50 mM MgCl₂, pH 9.5), and then incubated in detection buffer containing 0.175 mg/ml NBT and 0.35 mg/ml BCIP. The reaction is terminated in TE, pH 8. Tissue sections are counterstained with 1 μg/ml DAPI and mounted in VECTASHIELD (Vector Laboratory, Burlingame Calif.).

With the [³⁵S]-UTP protocol, fresh cryosections, 7-10 microns thick, are fixed for 10-20 min in 4% paraformaldehyde, rinsed in PBS, incubated in 0.2 M HCl for 20 min, and washed in DEPC H₂O. Sections are then incubated in 2×SSC for 30 min at 60 C., washed in DEPC H₂O, and permeablized for 10 min with 10 μg/ml proteinase K in 25 mM Tris-HCl (pH 7.5) and 5 mM EDTA (pH 8.0). Following incubation in 0.2% glycine in PBS for 30 seconds, the sections are washed in PBS, fixed for 20 min at 4 C. in 4% paraformaldehyde in PBS, washed in PBS, washed in 0.1 M TEA (pH 8.0), and then acetylated in 0.25% acetic anhydride in TEA for 10 min. After another PBS wash, sections are dehydrated in an EtOH series (30%, 60%, 80%, 95%, 100%×2) and air dried. Sections are incubated overnight at 45-60 C. in a humidified chamber with 50-100 μl of hybridization buffer plus probe (10⁵ cmp/μl) per slide. Hybridization buffer contains 50% formamide, 0.3 M NaCl, 10 mM Tris-HCl (pH7.5), 5 mM EDTA (pH 8.0), 1×Denhardt's, 10% dextran sulfate, 1 mg/ml yeast tRNA, and 10 mM DTT.

The following day, the slides are washed in 5×SSC with 10 mM DTT at 50 C. for 45 min, 2×SSC with 10 mM DTT at 65 C. for 20-30 min, and then in 0.5 M NaCl, 10 mM Tris-HCl (pH 7.5), 5 mM EDTA (pH 8.0) at 37 C. three times for 10 min each. Sections are incubated in the above solution with 20 μg/ml RNAse A at 37 C. for 30 min and rinsed in 2×SSC with 20 mM DTT at 65 C. for 20-30 min. Slides are then dehydrated in an EtOH series (30% in 300 mM NH₄OAc, 60%, 80%, 95%, 100%×2), air dried, and dipped in Kodak NT-B2 emulsion.

After six days, slides are developed, counterstained with hematoxylin and/or eosin, dehydrated and mounted with Permount (ProSciTech, Queensland Australia). Probes are labeled with [³⁵S]-UTP by in vitro transcription with either T7 (antisense) or T3 (sense) RNA polymerase using 320 or 370 basepair fragment (nucleotide x to y, or x′ to y′) using the cDNA of SEQ ID NO:1 as the template. The PCR-generated templates are each produced using two primers, one that contained the sequence for the T3 RNA polymerase promoter, and the other the sequence for the T7 RNA polymerase promoter. The two probes gave comparable results.

Membrane-based Hybridization

Membranes are pre-hybridized in hybridization solution containing 1% Sarkosyl and 1×high phosphate buffer (0.5 M NaCl, 0.1 M Na₂HPO₄, 5 mM EDTA, pH 7) at 55 C. for two hr. The probe, diluted in 15 ml fresh hybridization solution, is then added to the membrane. The membrane is hybridized with the probe at 55 C. for 16 hr. Following hybridization, the membrane is washed for 15 min at 25 C. in 1 mM Tris (pH 8.0), 1% Sarkosyl, and four times for 15 min each at 25 C. in 1 mM Tris (pH 8.0). To detect hybridization complexes, XOMAT-AR film (Eastman Kodak, Rochester N.Y.) is exposed to the membrane overnight at −70 C., developed, and examined visually.

Polymer Coated Slide-based Hybridization

The following method was used in the microarray analysis presented in Table 3. Probe is heated to 65 C. for five min, centrifuged five min at 9400 rpm in a 5415 C. microcentrifuge (Eppendorf Scientific, Westbury N.Y.), and then 18 μl is aliquoted onto the array surface and covered with a coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hr at 60 C. The arrays are washed for 10 min at 45 C. in 1×SSC, 0.1% SDS, and three times for 10 min each at 45 C. in 0.1×SSC, and dried.

Hybridization reactions are performed in absolute or differential hybridization formats. In the absolute hybridization format, probe from one sample is hybridized to array elements, and signals are detected after hybridization complexes form. Signal strength correlates with probe mRNA levels in the sample. In the differential hybridization format, differential expression of a set of genes in two biological samples is analyzed. Probes from the two samples are prepared and labeled with different labeling moieties. A mixture of the two labeled probes is hybridized to the array elements, and signals are examined under conditions in which the emissions from the two different labels are individually detectable. Elements on the array that are hybridized to equal numbers of probes derived from both biological samples give a distinct combined fluorescence (Shalon WO95/35505).

Hybridization complexes are detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light is focused on the array using a 20× microscope objective (Nikon, Melville N.Y.). The slide containing the array is placed on a computer-controlled X-Y stage on the microscope and raster-scanned past the objective with a resolution of 20 micrometers. In the differential hybridization format, the two fluorophores are sequentially excited by the laser. Emitted light is split, based on wavelength, into two photomultiplier tube detectors (PMT R1477, Hamamatsu Photonics Systems, Bridgewater N.J.) corresponding to the two fluorophores. Filters positioned between the array and the photomultiplier tubes are used to separate the signals. The emission maxima of the fluorophores used are 565 nm for Cy3 and 650 nm for Cy5. The sensitivity of the scans is calibrated using the signal intensity generated by the yeast control mRNAs added to the probe mix. A specific location on the array contains a complementary DNA sequence, allowing the intensity of the signal at that location to be correlated with a weight ratio of hybridizing species of 1:100,000.

The output of the photomultiplier tube is digitized using a 12-bit RTI-835H analog-to-digital (A/D) conversion board (Analog Devices, Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using the emission spectrum for each fluorophore. A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS program (Incyte Genomics).

QPCR Analysis

For QPCR, cDNA is synthesized from 1 ug total RNA in a 25 ul reaction with 100 units M-MLV reverse transcriptase (Ambion, Austin Tex.), 0.5 mM dNTPs (Epicentre, Madison Wis.), and 40 ng/ml random hexamers (Fisher Scientific, Chicago Ill.). Reactions are incubated at 25 C. for 10 minutes, 42 C. for 50 minutes, and 70 C. for 15 minutes, diluted to 500 ul, and stored at −30 C. Alternatively, normal tissues are purchased from Clontech (Palo Alto Calif.) and Clinomics. PCR primers and probes (5′ 6-FAM-labeled, 3′ TAMRA) are designed using PRIMER EXPRESS 1.5 software (ABI) and synthesized by Biosearch Technologies (Novato Calif.) or ABI.

QPCR reactions are performed using an PRISM 7700 detection system (ABI) in 25 ul total volume with 5 ul cDNA template, 1×TAQMAN UNIVERSAL PCR master mix (ABI), 100 nM each PCR primer, 200 nM probe, and 1×VIC-labeled beta-2-microglobulin endogenous control (ABI). Reactions are incubated at 50 for 2 minutes, 95 C. for 10 minutes, followed by 40 cycles of incubation at 95 C. for 15 seconds and 60 C. for 1 minute. Emissions are measured once every cycle, and results are analyzed using SEQUENCE DETECTOR 1.7 software (ABI) and fold differences, relative concentration of mRNA as compared to standards, are calculated using the comparative C_{T} method (ABI User Bulletin #2). QPCR is used to produce the data for FIGS. 3, 4, and 5

VIII Complementary Molecules

Antisense molecules complementary to the cDNA, from about 5 bp to about 5000 bp in length, are used to detect or inhibit gene expression. Detection is described in Example VII. To inhibit transcription by preventing promoter binding, the complementary molecule is designed to bind to the most unique 5′ sequence and includes nucleotides of the 5′ UTR upstream of the initiation codon of the open reading frame. Complementary molecules include genomic sequences (such as enhancers or introns) and are used in triple helix base pairing to compromise the ability of the double helix to open sufficiently for the binding of polymerases, transcription factors, or regulatory molecules. To inhibit translation, a complementary molecule is designed to prevent ribosomal binding to the mRNA encoding the protein.

Complementary molecules are placed in expression vectors and used to transform a cell line to test efficacy; into an organ, tumor, synovial cavity, or the vascular system for transient or short term therapy; or into a stem cell, zygote, or other reproducing lineage for long term or stable gene therapy. Transient expression lasts for a month or more with a non-replicating vector and for three months or more if elements for inducing vector replication are used in the transformation/expression system.

Stable transformation of dividing cells with a vector encoding the complementary molecule produces a transgenic cell line, tissue, or organism (U.S. Pat. No. 4,736,866). Those cells that assimilate and replicate sufficient quantities of the vector to allow stable integration also produce enough complementary molecules to compromise or entirely eliminate activity of the cDNA encoding the protein.

IX Expression of PDE8s

A 1.6 kb region of PDE8A(E) (the C-terminal 545 amino acids) was amplified and cloned into the baculovirus transfer vector pFASTBAC (Invitrogen), which had been modified to include a 5′ FLAG tag. Recombinant virus stocks were prepared according to the manufacturer's protocol. Sf9 cells were cultured in Sf900 II Sfm serum free media (Invitrogen) at 27 C. For expression, 1×10⁸ Sf9 cells were infected at a multiplicity of infection of 5 in a final volume of 50 mls. At three days post-infection, the cells were harvested, and enzyme-containing lysates were prepared as described below.

To monitor expression, 1 ml each of mock-infected and PDE8A(E) infected cell lysate was electrophoresed in a polyacrylamide gel and either silver-stained by standard methods or transferred to nitrocellulose and assayed using western analysis and anti-FLAG M2 antibodies (Sigma-Aldrich) at a concentration of 2 mg/ml. The secondary antibody was an alkaline phosphatase conjugated anit-mouse IgG (Boehringer Mannheim, Indianapolis Ind.). The blot was visualized with a BCIP/NBT phosphatase substrate system (Kirkegaard & Perry Laboratories, Gaithersburg, Md.) according to the manufacturer's protocol.

PDE8A(E) to be used for assay was prepared from transfected Sf9 cells. Cells were harvested by centrifugation, resuspended in homogenization buffer (20 mM Tris-HCl, 2 mM benzamidine, 1 mM EDTA, 0.25 M sucrose, 100 uM PMSF, pH 7.5) at 1×10⁷ cells/ml, and disrupted with 3×10 second pulses using a Sonifier Processor (Emerson Branson, Korea). Cellular debris was removed by centrifugation at 14,000×g for 10 minutes. The supernatant was stored at −70° C.

X Demonstration of PDE8 Activity

PDE activity was assayed by measuring the conversion of ³H-cAMP to ³H-adenosine in the presence of PDE8A(E) and 5′ nucleotidase. A one-step assay was run using a 100 μl assay containing 50 mM Tris-HCl, pH 7.5, 10 mM MgCl², 0.1 unit 5′ nucleotidase (from Crotalus atrox venom), 0.0062-0.1 μM ³H-cAMP, and various concentrations of cAMP (0.0062-3 mM). The reaction was started by the addition of 25 ul of diluted enzyme supernatant. Reactions were run directly in mini Poly-Q scintillation vials (BeckmanCoulter, Fullerton Calif.). Assays were incubated at 37 C. for a time period that would give less than 15% cAMP hydrolysis to avoid non-linearity associated with product inhibition. The reaction was stopped by the addition of 1 ml of Dowex AG1×8 (C1 form) resin (1:3 slurry). Three ml of scintillation fluid were added, the contents of the vials were mixed, and the resin in the vials was allowed to settle for 1 hr before counting. Soluble radioactivity associated with ³H-adenosine was quantitated using a Beta scintillation counter. The amount of radioactivity recovered is proportional to the activity of PDE8 in the reaction. For inhibitor studies (FIG. 8), all reactions contained 1% DMSO, 50 nM cAMP, and the indicated inhibitor concentrations. The control vial contained all reagents minus the enzyme aliquot.

XI Production of Specific Antibodies

The amino acid sequences of the PDE8s were analyzed using MACDNASIS (Hitachi Software Engineering) and/or LASERGENE software (DNASTAR) to determine regions of high immunogenicity. Oligopeptides extending from residue Q₆₃₅ to residue P₆₄₉ or from residue S²²⁰ to about residue R₂₃₉ of SEQ ID NO:1 and from residue H₂₂₇ to about residue R₂₄₆ of SEQ ID NO:8 are synthesized and conjugated to KLH (Sigma-Aldrich).

Rabbits are injected with the oligopeptide-KLH complexes in complete Freund's adjuvant, and the resulting antisera were tested for specific recognition of PDE8s as shown in FIGS. 7 and 10. Antisera that reacted positively with PDE8s is affinity purified on a column containing beaded agarose resin to which the synthetic oligopeptide had been conjugated (Pierce Sulfolink kit; Pierce Chemical, Rockford Ill.). The column is equilibrated using 12 ml IMMUNOPURE Gentle Binding buffer (Pierce Chemical). Three ml of rabbit antisera is combined with one ml of binding buffer and added to the top of the column. The column is capped on the top and bottom, and antisera is allowed to bind with gentle shaking at room temperature for 30 min. The column is allowed to settle for 30 min, drained by gravity flow, and washed with 16 ml binding buffer (4×4 ml additions of buffer). The antibody is eluted in one ml fractions with IMMUNOPURE Gentle Elution buffer (Pierce), and absorbance at 280 nm is determined. Peak fractions are pooled and dialyzed against 50 mM Tris, pH 7.4, 100 mM NaCl, and 10% glycerol. After dialysis, the concentration of the purified antibody is determined using the BCA assay (Pierce), aliquotted, and frozen.

XII Immunopurification Using Antibodies

Recombinantly produced protein is purified by immunoaffinity chromatography using anti-FLAG M2 antibodies. An immunoaffinity column is constructed by covalently coupling the antibody to CNBr-activated agarose resin (Sigma-Aldrich). Media containing the protein is passed over the immunoaffinity column, and the column is washed using high ionic strength buffers in the presence of detergent to allow preferential absorbance of the protein. After coupling, the protein is eluted with 1 ml aliquots of 0.1M glycine, pH 2.5, into vials containing 1 M Tris, pH 8.3. FIG. 7 shows immunopurified PDE8s.

XIII Western Analysis and Immunohistochemistry

Electrophoresis and Blotting

Samples containing protein are mixed in 2×loading buffer, heated to 95 C. for 3-5 min, and loaded on 4-12% NUPAGE Bis-Tris precast gel (Invitrogen). Unless indicated, equal amounts of total protein are loaded into each well. The gel is electrophoresced in 1×MES or MOPS running buffer (Invitrogen) at 200 V for approximately 45 min on an Xcell II apparatus (Invitrogen) until the RAINBOW marker (APB) had resolved, and dye front approached the bottom of the gel. The gel and its supports are removed from the apparatus and soaked in 1×transfer buffer (Invitrogen) with 10% methanol for a few minutes; and the PVDF membrane is soaked in 100% methanol for a few seconds to activate it. The membrane, the gel, and supports are placed on the TRANSBLOT SD transfer apparatus (Biorad, Hercules Calif.) and a constant current of 350 mAmps is applied for 90 min.

Conjugation with Antibody and Visualization

After the proteins are transferred to the membrane, it is blocked in 5% (w/v) non-fat dry milk in 1×phosphate buffered saline (PBS) with 0.1% Tween 20 detergent (blocking buffer) on a rotary shaker for at least 1 hr at room temperature or at 4 C. overnight. After blocking, the buffer is removed, and 10 ml of primary antibody in blocking buffer is added and incubated on the rotary shaker for 1 hr at room temperature or overnight at 4 C. The membrane is washed 3× for 10 min each with PBS-Tween (PBST), and secondary antibody, conjugated to horseradish peroxidase, is added at a 1:3000 dilution in 10 ml blocking buffer. The membrane and solution are shaken for 30 min at room temperature and then washed three times for 10 min each with PBST.

The wash solution is carefully removed, and the membrane is moistened with ECL+ chemiluminescent detection system (APB) and incubated for approximately 5 min. The membrane, protein side down, is placed on BIOMAX M film (Eastman Kodak) and developed for approximately 30 seconds.

XIV Antibody Arrays

Protein:Protein Interactions

In an alternative to yeast two hybrid system analysis of proteins, an antibody array can be used to study protein-protein interactions and phosphorylation. A variety of protein ligands are immobilized on a membrane using methods well known in the art. The array is incubated in the presence of cell lysate until protein:antibody complexes are formed. Proteins of interest are identified by exposing the membrane to an antibody specific to the protein of interest. In the alternative, a protein of interest is labeled with digoxigenin (DIG) and exposed to the membrane; then the membrane is exposed to anti-DIG antibody which reveals where the protein of interest forms a complex. The identity of the proteins with which the protein of interest interacts is determined by the position of the protein of interest on the membrane.

Proteomic Profiles

Antibody arrays can also be used for high-throughput screening of recombinant antibodies. Bacteria containing antibody genes are robotically-picked and gridded at high density (up to 18,342 different double-spotted clones) on a filter. Up to 15 antigens at a time are used to screen for clones to identify those that express binding antibody fragments. These antibody arrays can also be used to identify proteins which are differentially expressed in samples (de Wildt, supra)

XV Screening Molecules for Specific Binding with the cDNA or Protein

The cDNAs of SEQ ID NOs:2-6 and 9-15, or fragments thereof, or the protein, or portions thereof, are labeled with ³²P-dCTP, Cy3-dCTP, or Cy5-dCTP (APB), or with BIODIPY or FITC (Molecular Probes), respectively. Libraries of candidate molecules or compounds previously arranged on a substrate are incubated in the presence of labeled cDNA or protein. After incubation under conditions for either a nucleic acid or amino acid sequence, the substrate is washed, and any position on the substrate retaining label, which indicates specific binding or complex formation, is assayed, and the ligand is identified. Data obtained using different concentrations of the nucleic acid or protein are used to calculate affinity between the labeled nucleic acid or protein and the bound molecule.

XVI Two-Hybrid Screen

A yeast two-hybrid system, MATCHMAKER LexA Two-Hybrid system (Clontech Laboratories), is used to screen for peptides that bind the protein of the invention. A cDNA encoding the protein is inserted into the multiple cloning site of a pLexA vector, ligated, and transformed into E. coli. cDNA, prepared from mRNA, is inserted into the multiple cloning site of a pB42AD vector, ligated, and transformed into E. coli to construct a cDNA library. The pLexA plasmid and pB42AD-cDNA library constructs are isolated from E. coli and used in a 2:1 ratio to co-transform competent yeast EGY48[p8op-lacZ] cells using a polyethylene glycol/lithium acetate protocol. Transformed yeast cells are plated on synthetic dropout (SD) media lacking histidine (-His), tryptophan (-Trp), and uracil (-Ura), and incubated at 30 C. until the colonies have grown up and are counted. The colonies are pooled in a minimal volume of 1×TE (pH 7.5), replated on SD/-His/-Leu/-Trp/-Ura media supplemented with 2% galactose (Gal), 1% raffinose (Raf), and 80 mg/ml 5-bromo-4-chloro-3-indolyl β-d-galactopyranoside (X-Gal), and subsequently examined for growth of blue colonies. Interaction between expressed protein and cDNA fusion proteins activates expression of a LEU2 reporter gene in EGY48 and produces colony growth on media lacking leucine (-Leu). Interaction also activates expression of β-galactosidase from the p8op-lacZ reporter construct that produces blue color in colonies grown on X-Gal.

Positive interactions between expressed protein and cDNA fusion proteins are verified by isolating individual positive colonies and growing them in SD/-Trp/-Ura liquid medium for 1 to 2 days at 30 C. A sample of the culture is plated on SD/-Trp/-Ura media and incubated at 30 C. until colonies appear. The sample is replica-plated on SD/-Trp/-Ura and SD/-His/-Trp/-Ura plates. Colonies that grow on SD containing histidine but not on media lacking histidine have lost the pLexA plasmid. Histidine-requiring colonies are grown on SD/Gal/Raf/X-Gal/-Trp/-Ura, and white colonies are isolated and propagated. The pB42AD-cDNA plasmid, which contains a cDNA encoding a protein that physically interacts with the protein, is isolated from the yeast cells and characterized.

All patents and publications mentioned in the specification are incorporated by reference herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

What is claimed is:

1. An isolated antibody that specifically binds a protein having the amino acid sequence of SEQ ID NO:1 or SEQ ID NO:8 or an epitope extending from residue Q₆₃₅ to residue P₆₄₉ or from residue S₂₂₀ to residue R₂₃₉ of SEQ ID NO:1 or from residue H₂₂₇ to residue R₂₄₆ of SEQ ID NO:8.

2. A monoclonal antibody having the specificity of the antibody of claim 1.

3. The antibody of claim 1 wherein the antibody is identified with the protein having the amino acid sequence of SEQ ID NO:1 or an epitope extending from residue Q₆₃₅ to residue P₆₄₉ or from residue S₂₂₀ to residue R₂₃₉ of SEQ ID NO:1 by screening a plurality of intact immunoglobulin molecules, chimeric antibodies, polyclonal antibodies, recombinant antibodies, humanized antibodies, single chain antibodies, Fab fragments, F(ab′)₂ fragments, or Fv fragments.

4. A method for using an antibody to detect differential expression of a protein in a sample, the method comprising:
a) combining the antibody of claim 1 with a sample under conditions which allow the formation of antibody:protein complexes; and
b) detecting complex formation, wherein complex formation indicates differential expression of the protein in the sample.

5. The method of claim 4 wherein the sample is from prostate, pancreas or lung.

6. The method of claim 4 wherein complex formation is compared with at least one standard and is used to stage prostate cancer.

7. A composition comprising an antibody of claim 1 and a labeling moiety.

8. A kit comprising the composition of claim 1.

9. An array element comprising the antibody of claim 1.

10. A substrate upon which the antibody of claim 1 is immobilized.

11. A composition comprising the antibody of claim 1 and a pharmaceutical agent.

12. The composition of claim 11 wherein the composition is formulated to function in the prostate.

13. The composition of claim 11 wherein the pharmaceutical agent is a cytotoxic compound.

14. A method for using a composition to assess efficacy of a small drug molecule, the method comprising:
a) treating a sample containing the protein having the amino acid sequence of SEQ ID NO:1 with a molecule;
b) contacting the protein in the sample with the composition of claim 7 under conditions for complex formation; and
c) comparing the amount of complex formation in the treated sample with the amount of complex formation in an untreated sample, wherein a difference in complex formation indicates efficacy of the molecule.

15. A method for using a composition to assess toxicity of a compound, the method comprising:
a) treating a sample containing the protein having the amino acid sequence of SEQ ID NO:1 with a compound;
b) contacting the protein in the sample with the composition of claim 7 under conditions for complex formation; and
c) comparing the amount of complex formation in the treated sample with the amount of complex formation in an untreated sample, wherein a difference in complex formation indicates toxicity of the compound.

16. A method for treating prostate cancer comprising administering to a subject in need of therapeutic intervention the antibody of claim 1.

17. A method for treating prostate cancer comprising administering to a subject in need of therapeutic intervention the antibody of claim 2.

18. A method for treating prostate cancer comprising administering to a subject in need of therapeutic intervention the composition of claim 11.

19. A method for delivering an antibody to a subject in need of therapeutic intervention, wherein the antibody is formulated injection.

20. A method for delivering a pharmaceutical agent to the prostate comprising administering the composition of claim 11 to a subject in need of therapeutic intervention, wherein the composition specifically binds the protein having the amino acid sequence of SEQ ID NO:1 or SEQ ID NO:8 thereby delivering the pharmaceutical agent.

21. The antibody of claim 1 wherein the antibody is identified with the protein having the amino acid sequence of SEQ ID NO:8 or an epitope from residue H₂₂₇ to residue R₂₄₆ of SEQ ID NO:8 by screening a plurality of intact immunoglobulin molecules, chimeric antibodies, polyclonal antibodies, recombinant antibodies, humanized antibodies, single chain antibodies, Fab fragments, F(ab′)₂ fragments, or Fv fragments.


SEQUENCE LISTING (26 sequences)

SEQ ID NO: 1 - protein, 713 aa, Homo sapiens
  misc_feature: Incyte ID No 156196CD1
LACFLDKHHDIIIIDHRNPRQLDAEALCRSIRSSKLSENTVIVGVVRRVDREELSVMPFISAGFTRRYVENPNIMACYNELLQLEFGEVRSQLKLRACNSVFTALENSEDAIEITSEDRFIQYANPAFETTMGYQSGELIGKELGEVPINEKKADLLDTINSCIRIGKEWQGIYYAKKKNGDNIQQNVKIIPVIGQGGKIRHYVSIIRVCNGNNKAEKISECVQSDTRTDNQTGKHKDRRKGSLDVKAVASRATEVSSQRRHSSMARIHSMTIEAPITKVINVINAAQESSPMPVTEALDRVLEILRTTELYSPQFGAKDDDPHANDLVGGLMSDGLRRLSGNEYVLSTKNTQMVSSNIITPISLDDVPPRIARAMENEEYWDFDIFELEAATHNRPLIYLGLKMFARFGICEFLHCSESTLRSWLQIIEANYHSSNPYHNSTHSADVLHATAYFLSKERIKETLDPIDEVAALIAATIHDVDHPGRTNSFLCNAGSELAILYNDTAVLESHHAALAFQLTTGDDKCNIFKNMERNDYRTLRQGIIDMVLATEMTKHFEHVNKFVNSINKPLATLEENGETDKNQEVINTMLRTPENRTLIKRMLIKCADVSNPCRPLQYCIEWAARISEEYFSQTDEEKQQGLPVVMPVFDRNTCSIPKSQISFIDYFITDMFDAWDAFVDLPDLMQHLDNNFKYWKGLDEMKLRNLRPPPE

SEQ ID NO: 2 - DNA, 3396 bp, Homo sapiens
  misc_feature: Incyte ID No 156196CB1
cttgcctgtttcctggacaaacatcatgacattatcatcatagaccacagaaatcctcgacagctggatgcagaggcactgtgcaggtctatcagatcatcaaaactctcagaaaacacagttattgttggtgtagtacgcagggtggatagagaagagttgtccgtaatgcctttcatttctgctggatttacaaggaggtatgtagaaaaccccaacatcatggcctgctacaatgaactgctccagctggagtttggagaggtgcgatcacaactgaaactcagggcttgtaactcagtattcactgcattagaaaacagtgaagatgcaattgaaattacaagcgaagaccgttttatacagtatgcaaatcctgcatttgaaacaacaatgggctatcagtcaggtgaattaatagggaaggagttaggagaagtgcctataaatgaaaaaaaggctgacttgctcgatactataaattcatgcatcaggataggcaaggagtggcaaggaatttactatgccaaaaagaaaaacggagataatatacaacaaaatgtgaagataatacctgtcattggacagggaggaaaaattagacactatgtgtccattatcagagtgtgcaatggcaacaataaggctgagaaaatatccgaatgtgttcagtctgacactcgtacagataatcagacaggcaaacataaagacaggagaaaaggctcactagacgtcaaagctgttgcctcccgtgcaactgaagtttccagccagagacgacactcttccatggcccggatacattccatgacaattgaggcgcccatcaccaaggtaatcaatgttatcaatgctgcccaggaaagtagtcccatgcctgtgacagaagccctagaccgtgtgctggaaattctaagaaccactgagttatattcaccacagtttggtgctaaagatgatgatccccatgccaatgaccttgttgggggcttaatgtctgatggtttgcgaagactatcagggaatgaatatgttctttcaacaaaaaacactcaaatggtttcaagcaatataatcactcccatctcccttgatgatgtcccaccacggatagctcgggccatggaaaatgaggaatactgggactttgatatttttgaactggaggctgccacccacaataggcctttgatttatcttggtctcaaaatgtttgctcgctttggaatctgtgaattcttacactgctccgagtcaacgctaagatcatggttacaaattatcgaagccaattatcattcctccaatccctaccacaattctacacattctgctgatgtgcttcatgccactgcctattttctctccaaggagaggataaaggaaactttagatccaattgatgaggtcgctgcactcatcgcagccaccattcatgatgtggatcaccctgggagaaccaactccttcctgtgtaatgctggaagtgagctggccattttgtacaatgacactgctgtgctggagagccaccatgcggccttggccttccagctgaccactggagatgataaatgcaatatatttaaaaacatggagaggaatgattatcggacactgcgccaggggattatcgacatggtcttagccacagaaatgacaaagcactttgagcatgtcaacaaatttgtcaacagcatcaacaaacccttggcaacactagaagaaaatggggaaactgataaaaaccaggaagtgataaacactatgcttaggactccagagaaccggaccctaatcaaacgaatgctgattaaatgtgctgatgtgtccaatccctgccgacccctgcagtactgcatcgagtgggctgcacgcatttcggaagaatatttttctcagactgatgaagagaagcagcagggcttacctgtggtgatgccagtgtttgacagaaatacctgcagcatccccaaatcccaaatctctttcattgattacttcatcacagacatgtttgatgcttgggatgcctttgtagacctgcctgatttaatgcagcatcttgacaacaactttaaatactggaaaggactggacgaaatgaagctgcggaacctccgaccacctcctgaatagtgggagacaccacccagagccctgaagctttgttccttcggtcatttggaattcctgagggcagccagagctccttggtcctttcagtactaggcagaacagcccccgatctgcatagcctgtgaaagcccacggggacatcagtaaccttctgcagccaccatccaatgccattactgtcaagtgagacttggccactgtagcctgggcctgctgcaggagctcttcagaaaggcacatgaggaccacggtttgcctcagtttctggtaaaacacaaggtctggagtgcccctgcaaagggtattgatggacttcctgccagtgacagagcatgtctattgcaaacaattctctcagttacgttcagcacttaagaacggctaatggcaataggatctttagcaactttttcacatcatagaaggtgcaatcgctcacttgggaacactactgagagtgacttctcttttaaaattgagtagcagatgaaaaattaaaatttgaacttgattattaatatcaattaaaatgttttatttattttattaaaagctcaatattttctatgaattcaaaaatacttcagagccaaagccaacttcaaataccgtgaccaaatttacatgattcatattcattatgcattacttggtatacagacttattttcataatgcaaattaataaaatgacacttttactgcactatagaaatattcatgtatgttaaacttttctgattgaggctaactggaaaaagctggggtcgtattctaagtgctaaagaaggctgcttctactgtatagaacccagggctctgaaacagctctagccgcctaatgcacttcacaggtaactccccaaggtaaaactagactctcttgttggttcgcaaagaaaagttaggacttaacacttttttctaaaattttataattcaatttccaaaagtctactctattttatactgtttctacaaaatattccttataaaaacaaagaacaaaaattgaatatttaatgaattgacattttataaccaacctgtttttatctacggtgggaatctttgatgccagaaatttataaagaggttctgtatcttcacaccttgaataagcataataccataaaaaatgacacttgacatgtcaatgtatttgtcatttcattttaaactcgtatttgtggtttttttcccagataaaaatgaaattaaaccatttctttttaagaaaaaaaaaaaaaaaaaa

SEQ ID NO: 3 - DNA, 2201 bp, Homo sapiens
  misc_feature: Incyte ID No 156196
cgatactataaattcatgcatcaggataggcaaggagtggcaaggaatttactatgccaaaaagaaaaacggagataatatacaacaaaatgtgaagataatacctgtcattggacagggaggaaaaattagacactatgtgtccattatcagagtgtgcaatggcaacaataaggctgagaaaatatccgaatgtgttcagtctgacactcatacagataatcagacaggcaaacataaagacaggagaaaaggctcactagacgtcaaagctgttgcctcccgtgcaactgaagtttccagccagagacgacactcttccatggcccggatacattccatgacaattgaggcgcccatcaccaaggtaatcaatattatcaatgctgcccaggaaagtagtcccatgcctgtgacagaagccctagaccgtgtgctggaaattctaagaaccactgagttatattcaccacagtttggtgctaaagatgatgatccccatgccaatgaccttgttgggggcttaatgtctgatggtttgcgaagactatcagggaatgaatatgttctttcaacaaaaaacactcaaatggtttcaagcaatataatcactcccatctcccttgatgatgtcccaccacggatagctcgggccatggaaaatgaggaatactgggactttgatatttttgaactggaggttgccacccacaataggcctttgatttatcttggtctcaaaatgtttgctcgctttggaatctgtgaattcttacactgctccgagtcaacgctaagatcatggttacaaattatcgaagccaattatcattcctccaatccctaccacaattctacacattctgctgatgtgcttcatgccactgcctattttctctccaaggagaggataaaggaaactttagatccaattgatgaggtcgctgcactcatcgcagccaccattcatgatgtggatcaccctgggagaaccaactccttcctgtgtaatgctggaagtgagctggccattttgtacaatgacactgctgtgctggagagccaccatgcggccttggccttccagctgaccactggagatgataaatgcaatatatttaaaaacatggagaggaatgattatcggacactgcgccaggggattatcgacatggtcttagccacagaaatgacaaggcactttgagcatgtcaacaaatttgtcaacagcatcaacaaacccttggcaacactagaagaaaatggggaaactgataaaaaccaggaagtgataaacactatgcttaggactccagagaaccggaccctaatcaaacgaatgctgattaaatgtgctgatgtgtccaatccctgccgacccctgcagtactgcatcgagtgggctgcacgcatttcggaagaatatttttctcagactgatgaagagaagcagcagggcttacctgtggtgatgccagtgtttgacagaaatacctgcagcatccccaaatcccaaatctctttcattgattacttcatcacagacatgtttgatgcttgggatgcctttgtagacctgcctgatttaatgcagcatcttgacaacaactttaaatactggaaaggactggacgaaatgaagctgcggaacctccgaccacctcctgaatagtgggagacaccacccagagccctgaagctttgttccttcggtcatttggaattcctgagggcaraccagagctccttggtcctttcagtrcwaggcagnanacagcccccgatctgyatagcctgtgaaagcccrcggggacatcagtaaccttctkcagccaccatccaatgccattactgtcaagtgagacttggccmctgtaccctgggcctkctkcaggagctcttcagaaaggcacatkaggaccacggntttsgctcagtttctggtaaaacacaaggtctggagtkcccctgcmaagggtattgatggacttcctkccagtgacagagcatgtctatttccaacaattctctcanttacgttcaacacttaagaacggctaatggcaataggatctttaacaactttttcacatcanagnaggttcaatcgctcacttgggnacacnactgagagtgacttctcttttaaaattgagtaacagatggaaaaataaaatttggacttgattattaanatcccn

SEQ ID NO: 4 - DNA, 605 bp, Macaca fasciculata
  misc_feature: Incyte ID No 025320_Mf.1
tcacttggtatacagacttactcttgcattatgcaaaataacgtctgtataccaagtaactgcaaattaatataaaatgacatttttactgcactatagaaatattcgtgtatgttaaacttttctgattgaggctaattggcaaaaagctggggtctttttctaagcactaaagaaggctgcttctactctatagaacccagggctctgaaatagctctagctgcctaatgaacttcacaggtaactccccaaggtaaaattagactcttcttggtttgcaaagaaaaattaggacttaacacttttttctaaaattttataattgaattccaaacgtctaccctattttatactgtttctacaaaatattccttataaaaacaagaacaaaatggtatatttaatgaattgacattttataaccaacctgtttttatctgcagtgggaatctttgatgccagaaatttataaagaggttctgtaccttcacactttgaataagcataataccataaaaaatgacacttgacatgtcaatgtgtttgtcatttcattttaaactcgtatttgtggttttttcccagataaaaatgaaattaaac

SEQ ID NO: 5 - DNA, 706 bp, Canis familiaris
  misc_feature: Incyte ID No 052438_Cf.1
aggctcaggctgtccttgcctgtttcctggacaaacatcacgacattgtcatcattgaccacagaaacccccggcagctggacgcagaggcgctgtgcaggtctatcagatcctcagaactctctgaaaacacaattattattggtgtagtacggaggacggatagagaagagtcctctgtgatgcccctcattgctgctggctttacaaggaggtatgtggaaaaccccagcctcatggcctgttacaatgaactgctccagctggagttcggagaggtgcggtcccagctgaaactcagggcttgtaactcaatattcacggcattagagaaaagtcaagaagcaattgaaattacaagtgaagaccgcgttatacagtatgcaaatcctgcatttgaaacaaccatgggctatcagtcaggtgaattaataggaaaggagttagcagaagtgcctataaatgaaaaaaaggctgatttgctcgatactataaattcatgcatcaggataggcaaggagtggcaaggaatctaccatgccaaaaagaaaaatggagataatgtgcaacaaaatgtgaagataatacctgtcattggacagggagggtaagtggaaaatccaggtactgcagtcaacataccatgcttggcttagggcagaatntgctctgaatacttcagtaggttaagaactgcatgcagaaa

SEQ ID NO: 6 - DNA, 575 bp, Rattus norvegicus
  misc_feature: Incyte ID No 700310355F6
ccaaataggccttgatttatctaggtctcaaaacgttgctcgttttgaaatcgtgtgagttcttacagtgctctgaggcaacgctgaaatcatggtttcaaattattgaatccaattaccattcctctaacccctaccacaattccacgcacgccgcggatgtgcttcacgccactgcgtatttcctttccagaaacaagttaaaggacactttagatcgccttgatgaggtcgctgccctcatcgctgccaccatccacgatgtggatcaccccgggagaaccaactctttcctctgcaatgcgggaaaggagctggccgtactgtacaatgacactgctgtgctagagagccaccacgcagcgctggccttccagctgacccttgaaaatgacaaatgcaatatctttaaaaacacagacaggaatgactaccggacactacgccagagtatcacttgacatggtcctagccacagagatgacaaagcactttgaacacgtcaacaaatttatcaacagcatcaacaagcctctgaaagcacagaaacacgaggttcaggaaactggcagaag

SEQ ID NO: 7 - protein, 449 aa, Homo sapiens
  misc_feature: Incyte ID No 156196trun
MARIHSMTIEAPITKVINIINAAQESSPMPVTEALDRVLEILRTTELYSPQFGAKDDDPHANDLVGGLMSDGLRRLSGNEYVLSTKNTQMVSSNIITPISLDDVPPRIARAMENEEYWDFDIFELEVATHNRPLIYLGLKMFARFGICEFLHCSESTLRSWLQIIEANYHSSNPYHNSTHSADVLHATAYFLSKERIKETLDPIDEVAALIAATIHDVDHPGRTNSFLCNAGSELAILYNDTAVLESHHAALAFQLTTGDDKCNIFKNMERNDYRTLRQGIIDMVLATEMTRHFEHVNKFVNSINKPLATLEENGETDKNQEVINTMLRTPENRTLIKRMLIKCADVSNPCRPLQYCIEWAARISEEYFSQTDEEKQQGLPVVMPVFDRNTCSIPKSQISFIDYFITDMFDAWDAFVDLPDLMQHLDNNFKYWKGLDEMKLRNLRPPPE

SEQ ID NO: 8 - protein, 718 aa, Homo sapiens
  misc_feature: Incyte ID No 464655CD1
ALECFLDKHHEIIVIDHRQTQNFDAEAVCRSIRATNPSEHTVILAVVSRVSDDHEEASVLPLLHAGFNRRFMENSSIIACYNELIQIEHGEVRSQFKLRACNSVFTALDHCHEAIEITSDDHVIQYVNPAFERMMGYHKGELLGKELADLPKSDKNRADLLDTINTCIKKGKEWQGVYYARRKSGDSIQQHVKITPVIGQGGKIRHFVSLKKLCCTTDNNKQIHKIHRDSGDNSQTEPHSFRYKNRRKESIDVKSISSRGSDAPSLQNRRYPSMARIHSMTIEAPITKVINIINAAQENSPVTVAEALDRVLEILRTTELYSPQLGTKDEDPHTSDLVGGLMTDGLRRLSGNEYVFTKNVHQSHSHLAMPITINDVPPCISQLLDNEESWDFNIFELEAITHKRPLVYLGLKVFSRFGVCEFLNCSETTLRAWFQVIEANYHSSNAYHNSTHAADVLHATAFFLGKERVKGSLDQLDEVAALIAATVHDVDHPGRTNSFLCNAGSELAVLYNDTAVLESHHTALAFQLTVKDTKCNIFKNIDRNHYRTLRQAIIDMVLATEMTKHFEHVNKFVNSINKPMAAEIEGSDCECNPAGKNFPENQILIKRMMIKCADVANPCRPLDLCIEWAGRISEEYFAQTDEEKRQGLPVVMPVFDRNTCSIPKSQISFIDYFITDMFDAWDAFAHLPALMQHLADNYKHWKTLDDLKCKSLRLPSDS

SEQ ID NO: 9 - DNA, 2657 bp, Homo sapiens
  misc_feature: Incyte ID No 464655CB1
gcccttgaatgctttcttgataagcatcatgaaattattgtaattgatcatagacaaactcagaacttcgatgcagaagcagtgtgcaggtcgatccgggccacaaatccctccgagcacacggtgatcctcgcagtggtttcgcgagtatcggatgaccatgaagaggcgtcagtccttcctcttctccacgcaggcttcaacaggagatttatggagaatagcagcataattgcttgctataatgaactgattcaaatagaacatggggaagttcgctcccagttcaaattacgggcctgtaattcagtgtttacagcattagatcactgtcatgaagccatagaaataacaagcgatgaccacgtgattcagtatgtcaacccagccttcgaaaggatgatgggctaccacaaaggtgagctcctgggaaaagaactcgctgatctgcccaaaagcgataagaaccgggcagaccttctcgacaccatcaatacatgcatcaagaagggaaaggagtggcagggggtttactatgccagacggaaatccggggacagcatccaacagcacgtgaagatcaccccagtgattggccaaggagggaaaattaggcattttgtctcgctcaagaaactgtgttgtaccactgacaataataagcagattcacaagattcatcgtgattcaggagacaattctcagacagagcctcattcattcagatataagaacaggaggaaagagtccattgacgtgaaatcgatatcatctcgaggcagtgatgcaccaagcctgcagaatcgtcgctatccgtccatggcgaggatccactccatgaccatcgaggctcccatcacaaaggttataaatataatcaatgcagcccaagaaaacagcccagtcacagtagcggaagccttggacagagttctagagattttacggaccacagaactgtactcccctcagctgggtaccaaagatgaagatccccacaccagtgatcttgttggaggcctgatgactgacggcttgagaagactgtcaggaaacgagtatgtgtttactaagaatgtgcaccagagtcacagtcaccttgcaatgccaataaccatcaatgatgttcccccttgtatctctcaattacttgataatgaggagagttgggacttcaacatctttgaattggaagccattacgcataaaaggccattggtttatctgggcttaaaggtcttctctcggtttggagtatgtgagtttttaaactgttctgaaaccactcttcgggcctggttccaagtgatcgaagccaactaccactcttccaatgcctaccacaactccacccatgctgccgacgtcctgcacgccaccgctttctttcttggaaaggaaagagtaaagggaagcctcgatcagttggatgaggtggcagccctcattgctgccacagtccatgacgtggatcacccgggaaggaccaactctttcctctgcaatgcaggcagtgagcttgctgtgctttacaatgacactgctgttctggagagtcaccacaccgccctggccttccagctcacggtcaaggacaccaaatgcaacattttcaagaatattgacaggaaccattatcgaacgctgcgccaggctattattgacatggttttggcaacagagatgacaaaacactttgaacatgtgaataagtttgtgaacagcatcaacaagccaatggcagctgagattgaaggcagcgactgtgaatgcaaccctgctgggaagaacttccctgaaaaccaaatcctgatcaaacgcatgatgattaagtgtgctgacgtggccaacccatgccgccccttggacctgtgcattgaatgggctgggaggatctctgaggagtattttgcacagactgatgaagagaagagacagggactacctgtggtgatgccagtgtttgaccggaatacctgtagcatccccaagtctcagatctctttcattgactacttcataacagacatgtttgatgcttgggatgcctttgcacatctgccagccctgatgcaacatttggctgacaactacaaacactggaagacactagatgacctaaagtgcaaaagtttgaggcttccatctgacagctaaagccaagccacagagggggcctcttgaccgacaaaggacactgtgaatcacagtagcgtaaacgagaggccttcctttctaatgacaatgacaggtattggtgaaggagctaatgtttaatatttgaccttgaatcattcaagtccccaaatttcattcttagaaagttatgttccatgaagaaaaatatatgttcttttgaatacttaatgacagaacaaatacttggcaaactcctttgctctgctgtcatcctgtgtacccttgtcaatccatggagctggttcactgtaactagcaggccacaggaagcaaagccttggtgcctgtgagctcatctcccaggatggtgactaagtagcttagctagtgatcagctcatcctttaccataaaagtcatcattgctgtttagcttgactgttttcctcaagaacatcgatctgaaggattcataaggagcttatctgaacagatttatctaaaaaaaaaaaaaaaaaa

SEQ ID NO: 10 - DNA, 245 bp, Homo sapiens
  misc_feature: Incyte ID No 464655
catcaacaagccaatggcagctgagattgaaggcagcgactgtgaatgcaaccctgctgggaagaacttccctgnaaaccaaatcctgatcaaangcatgatgattaagtgtgctgangnggncaacccatgccgacccttggacctgtgcattgaatgggctgggaggatctctgaggagtattttgcacagactgatgaagagaagagacagggactacctgtggtgatgncagtgtttgacc

SEQ ID NO: 11 - DNA, 249 bp, Macaca fasciculata
  misc_feature: Incyte ID No 003237_Mf.1
ggatagagtaaagggaagcctcgatcagctggattaggtggcagccctcattctggccacggtccatgacgtggatcacccgggaatgaccaactctttcctctgcaatgcaggcattgagcttgctgtgctctacaatgacactgctgttctggagagtcaccacaccgccctggctttccagctcaccgtcaaggacaccaaatgcaacattttcaagaattttgacaggagccattatcgaacgct

SEQ ID NO: 12 - DNA, 685 bp, Canis familiaris
  misc_feature: Incyte ID No 704105231J1
gggtagtaattggaggagtggtagttggcttcaatcacttggagccaggcccgaagaagtagtttcagagcagtgcaaaaattcacatactccaaagcgagaaaatacctttaagcccaggtaaaccaatggccttttatgtgtaacagcttccaattcaaagatgttgaactcccaactttcctcattatcaagtaattgagcgatacaaggggggacatcgttgatggttattggcatcgcaaggtgactgtgactctggtgcacattcttagtgaacacatactcgtttcctgacaaccttctcaagccatcagtcatcaggcctccaaccagatcactggtatggggatcttcatctttggtacccagctgaggggaatacagttctgtggtccgtaaaatttctaaaactctgtccaaggcttctgctactgtaacagggctgttttcttgggctgcattgattatatttataacctttgtgatgggagcctcgatggtcatggaatggatccttgccatggatggatagcgacgattctgtaggcttggtgcatcactgcctcgagatgatatcgatttcacatcaatggactctttccgtctgttcttgtatctgaatgaatgagactctgtctgaaaattgtctgctgaatcacgatgaaccttgtgaagctgctta

SEQ ID NO: 13 - DNA, 713 bp, Mus musculus
  misc_feature: Incyte ID No 143773_Mm.1
ggtcttcttttcggtttggagtatgtaaatttttaaactgtactgaaaccattctttcgggcctggttgcaagtgattgaagcccaattcccactcctccaacgcttaccacaactccacccatgctgccgatgtattgcatgccacggcttttttccttggaaaggaaagagtgaagggaagcctggaccagctggatgaggtggctgctctgattgctgccacagtccacgacgtggatcaccccggaaggaccaactctttcctgtgcaacgccggcagcgagctcgctgtgctctacaatgacactgcagtcttggagagtcaccacacagccctggccttccagctcacagtcaaggacaccaagtgcaacattttcaagaatattgacaggaaccactatcgaacgctgcgccaggctattatcgacatggttttggctacagagatgacaaaacacttcgagcacgtgaacaaattcgtgaacagcatcaacaagccactggcagctgagagtgagggcagcgattgtgaatgcaaccctactgggaagaactttcctgagaaccaaatcctcatcaaacgcatgatgattaagtgtgccgacgtggccaacccgtgccggcccttggacctgtgcattgaatgggctggaaggatctccgaggagtactttgctcagactgatgaagagaagaggcaggggctgc

SEQ ID NO: 14 - DNA, 990 bp, Mus musculus
  misc_feature: Incyte ID No 131862_Mm.1
gttactgtcagtggtacagcacagcttcttcagggagacaaagtgcctaattttccctccttggccaatcactggggtgatcttcacgtgctgttggatgctgtccccggatttccgtctggcatagtaaaccccctgccactcctttcccttcttgatgcacgtgttgatggtgtccaggaggtctgcccggttcttgtcactcttaggtaggtcagcgagttccttccccaggagctcccctttgtggtagcccatcatcctctcaaaggcagggttgacatactgaatcacatggtcatcacttgttatttctatggcttcgtgacagtggtctaacgctgtaaacactgaattacaggcccgtaatttgaactgtgagcgaacttccccgtgttctatttgaatcagttcattatagcaagcaattatgctgctgttctccataatctccggttgaagcctgcatgaagaagaggaaggactgaggcctcttcatggtcatcagatgcttgtgaaacgacagccaggatcaccgtgtgctcagaggggttcgtggcccggattgacctgcacacggcttctgcatcaaagttccggctctgcctgtgatcgatcacaataatttcatgatgcttgtccagaaagcactcgagggccgactctggagtgcgagcgatgttgcatctgtagccagctctgtcacaggcccaccagaagccatcactctggctatcttcctttgcgaagattagcagaacctggatagggtcctgtgtcagtctcatgggcccaatgcgcacctctgcagaagacacctggcgcacgctctgctgctggtccatacccgctgcgatccccagctgctgtcccaacgtgcaggtgcctgtgcccctagcagcagccgctcatggagcgcactagacagctgtacctttgcaccattaacctcgctctctctgcaagaagggtctggtcgctccaagggac

SEQ ID NO: 15 - DNA, 930 bp, Rattus norvegicus
  misc_feature: Incyte ID No 199543_Rn.1
ttccagctcaccgtcaaggacaccaagcgcaacattttcaagaatatcgacaggaaccactatcgaacgtctgcgccaggctattattgacatggttttggccacagagatggacgaaacactttgaacatgtgaacaagtttgtgaacagcatcaacaagcctctagcagccgagagtgagggcagcgactgtgaatgcaaccctactgggaagaactttcccgaaaaccaaatcctcatcaagcgtatgatgattaagtgtgctgacgtggccaacccgtgccgccccttggacctgtgcatcgaatgggctggaaggatctccgaggagtactttgcacagactgacgaagagaagaggcaggggctgcctgtagtgatgccagtgttcgaccgcaatacctgcagcatccccaagtcgcagatatccttcatcgactacttcataacagacatgtttgatgcttgggatgcctttgcacacctgccagccctgatgcaacacctggccgacaactataaacactggaagacactggatgacctcaagtgcaaaagtctgagacttccatctaacagctaaagccaagccagagaaggggacactgacctacagaggacactgtgaatcggagagtagtacaagccagagggtttctcttacggtggaaatgacaggcggacgttaaggagctagtgtttaatatctgacctgaatcatgcacgccccccaaatttcattttcagaaagttatgttccatcagtaaaaatatgtccttttgaatcacttagtgacaaaacgagccacttggcagaagaggctgcgagatgttagcctgtgtgcccttgtaaaaacccacagagctgacagatttgcggaaggcgcaagcaggacacaagacgcaaagacctgtgtctatgttgtccg

SEQ ID NO: 16 - protein, 81 aa, Homo sapiens
  misc_feature: Incyte ID No 464655trun
INKPMAAEIEGSDCECNPAGKNFPXNQILIKXMMIKCAXXXNPCRPLDLCIEWAGRISEEYFAQTDEEKRQGLPVVMXVFD

SEQ ID NO: 17 - protein, 610 aa, Rattus norvegicus
  misc_feature: GenBank ID No g1705952
MPLVDFFCETCSKPWLVGWWDQFKRMLNRELTHLSEMSRSGNQVSEYISNTFLDKQNEVEIPSPTPRQRAFQQPPPSVLRQSQPMSQITGLKKLVHTGSLNTNVPRFGVKTDQEDLLAQELENLSKWGLNIFCVSEYAGGRSLSCIMYTIFQERDLLKKFHIPVDTMMMYMLTLEDHYHADVAYHNSLHAADVLQSTHVLLATPALDAVFTDLEILAALFAAAIHDVDHPGVSNQFLINTNSELALMYNDESVLENHHLAVGFKLLQEENCDIFQNLSKRQRQSLRKMVIDMVLATDMSKHMTLLADLKTMVETKKVTSSGVLLLDNYSDRIQVLRNMVHCADLSNPTKPLELYRQWTDRIMAEFFQQGDRERERGMEISPMCDKHTASVEKSQVGFIDYIVHPLWETWADLVHPDAQDILDTLEDNRDWYHSAIRQSPSPPLEEEPGGLGHPSLPDKFQFELTLEEEEEEDSLEVPGLPTTEETFLAAEDARAQAVDWSKVKGPSTTVVEVAERLKQETASAYGAPQESMEAVGCSFSPGTPILPDVRTLSSSEEAPGLLGLPSTAAEVEAPRDHLAATRACSACSGTSGDNSAIISAPGRWGSGGDPA

SEQ ID NO: 18 - DNA, 18 bp, Homo sapiens
  misc_feature: Incyte ID No oligomer1
gaagcacatcagcagaat

SEQ ID NO: 19 - DNA, 21 bp, Homo sapiens
  misc_feature: Incyte ID No oligomer2
tcgcttagttttaccgttttc

SEQ ID NO: 20 - DNA, 22 bp, Homo sapiens
  misc_feature: Incyte ID No oligomer3
tatcgcctccatcaacaaactt

SEQ ID NO: 21 - DNA, 18 bp, Homo sapiens
  misc_feature: Incyte ID No oligomer4
ttgtggtagggattggag

SEQ ID NO: 22 - DNA, 21 bp, Homo sapiens
  misc_feature: Incyte ID No oligomer5
agcaagttcagcctggttaag

SEQ ID NO: 23 - DNA, 24 bp, Homo sapiens
  misc_feature: Incyte ID No oligomer6
cttatgagtatttcttccagggta

SEQ ID NO: 24 - DNA, 30 bp, Homo sapiens
  misc_feature: Incyte ID No oligomer7
atcatggttacaaattatcgaagccaatta

SEQ ID NO: 25 - protein, 10 aa, Homo sapiens
  misc_feature: Incyte ID No motif1
HDXXHXGXXN

SEQ ID NO: 26 - protein, 27 aa, Homo sapiens
  misc_feature: Incyte ID No motif2
HXXXHXXXXXXXXXXXXXXXXXXXXXE